(19) United States
(12) Patent Application Publication
(10) Pub. No.: US 2014/0010824 A1
(43) Pub. Date: Jan. 9, 2014

(54) Methods, compositions and kits for treating or preventing a disease associated with Gram-negative bacteria

(71) Applicant: Tufts University, Boston, MA (US)

(72) Inventors: Joan Mecsas, Needham, MA (US); Gregory T. Crimmins, Somerville, MA (US); Sina Mohammadi, Cambridge, MA (US); Erin R. Green, Cambridge, MA (US); Ralph R. Isberg, Newton Highlands, MA (US)

(21) Appl. No.: 13/912,850

(22) Filed: Jun. 7, 2013

Related U.S. Application Data
(60) Provisional application No. 61/656,640, filed on Jun. 7, 2012.

(51) Int. Cl. G01N 33/50

(52) U.S. Cl. CPC G01N 33/5005
USPC 424/164.1; 536/23.1; 536/24.5; 530/350; 530/389.5; 435/183; 514/44 R; 514/44 A; 424/94.1; 514/2.4; 424/93.7; 435/29; 435/6.15; 536/1.11

(57) ABSTRACT

Compositions, methods and kits are provided for identifying at least one virulence factor of a Gram-negative bacterial strain, and for preparing attenuated bacterial strain vaccines or a modulator that selectively binds to or inhibits expression of the virulence factor. The Gram-negative bacterial strain is a short facultatively aerobic or micro-aerobic rod or an enteric strain including at least one pathogen selected from the group of: Salmonella, Escherichia, Yersinia, Klebsiella, Shigella, Enterobacter, Serratia, Pseudomonas, and Citrobacter. Novel genes encoding virulence factors are identified, so that non-virulent mutant strains are available for vaccine development.

### RELATED APPLICATIONS

This utility application claims the benefit of U.S. provisional application Ser. No. 61/656,640 filed Jun. 7, 2012 entitled, “Methods, compositions and kits for treating or preventing a disease associated with Gram-negative bacteria” by Joan Mecsas, Gregory T. Crimmins, Sina Mohammadi, Erin R. Green and Ralph R. Isberg, which is incorporated by reference herein in its entirety.

### GOVERNMENT SUPPORT

This invention was made with government support under grants AI056068, AI085706, and AI023538 awarded by the National Institutes of Health. The government has certain rights in the invention.

### TECHNICAL FIELD

Methods, compositions and kits are provided for treating or preventing a disease associated with a Gram-negative bacterial strain, and for recombinantly producing a vaccine or a therapeutic agent.

### BACKGROUND

Gram-negative bacteria such as Yersinia are a major causative agent of disease including infections in the gastrointestinal system and lymph nodes. Wren, B. W. et al. 2003 Nature reviews Microbiology 1: 55-64; and Smego, R. A. et al. 1999 European Society of Clinical Microbiology 18: 1-15.

Genetic screens have been performed in pathogenic Yersinia species in pursuit of identifying virulence factors required during infection. Mecsas, J et al. 2001 Infection and immunity 69: 2779-2787; Darwin, A. J. et al. 1999 Miller Molecular microbiology 32: 51-62; Karlyshev, A. V. et al. Infection and immunity 69: 7810-7819; and Flashner, Y. et al. 2004 Infection and Immunity 72: 908-915. However, no systematic identification of virulence proteins has been achieved. Thus, there is a need for a detailed analysis of multiple chromosomal virulence factors that are critical for growth and persistence of Gram-negative bacteria in host cells and tissues.

### SUMMARY

An aspect of the invention provides a pharmaceutical composition for treating or preventing a disease associated with a Gram-negative bacterial strain, the composition including: a modulator of a virulence factor, such that the virulence factor is for example a protein, such that the modulator is specific to bind to the virulence factor to inhibit function or to bind to a gene encoding expression of the virulence factor, such that the composition prevents bacterial cell growth and infection of the Gram-negative strain in tissues of the subject.

In various embodiments of the composition, the Gram-negative bacterial strain is a short facultatively aerobic or micro-aerobic rod or an enteric strain, for example at least one pathogen selected from the group of Salmonella, Escherichia, Yersinia, Klebsiella, Shigella, Enterobacter, Serratia, Pseudomonas, and Citrobacter. In a related embodiment the strain is an Enterobacteriaceae. For example, the Gram-negative bacterial strain is Yersinia pseudotuberculosis, Yersinia pestis, or Yersinia enterocolitica. In various embodiments, the strain carries a virulence plasmid encoding a type III secretion system.

In various embodiments of the composition, the modulator includes a nucleic acid vector selected from DNA, mRNA, tRNA, rRNA, siRNA, RNAi, miRNA, and dsRNA, or a portion thereof. For example, the composition includes the vector including a nucleic acid sequence encoding the modulator that targets a virulence factor that is a non-polymorphic target having a conserved domain or a conserved region. Alternatively, the composition includes a vector including a nucleic acid sequence encoding the modulator which negatively modulates a polymorphic target. For example, the virulence factor is a genetic material (e.g., DNA or RNA) having at least one polymorphic nucleotide position in a population group. In various embodiments, the virulence factor has an amino acid sequence having at least one polymorphic amino acid residue in a population group.

In related embodiments of any of the above compositions, the vector is a viral vector or a plasmid, for example the viral vector is derived from a genetically engineered genome of at least one virus selected from the group consisting of: an adenovirus, an adeno-associated virus, a herpesvirus, and a lentivirus. For example, the lentivirus is a retrovirus.

In related embodiments, the vector is selected from the group consisting of: a lentivirus, an adeno-associated virus, and a helper-dependent adenovirus. The lentivirus for example includes a strain or a derivative of a human immunodeficiency virus, a simian immunodeficiency virus, a feline immunodeficiency virus, or an equine infectious anemia virus.

The modulator in various embodiments of the composition includes at least one selected from the group of: a protein for example an antibody or an enzyme, a carbohydrate for example a sugar, and a small molecule for example a drug for example an antibiotic. For example, the modulator is a fusion protein having a plurality of domains, for example a first domain inhibits the virulence factor and a second domain provides an additional therapeutic effector function. In certain embodiments, the modulator is a binding protein that includes and is composed of DNA-binding domains. For example, the binding protein has a specific or general affinity for either single stranded DNA or double stranded DNA. In certain embodiments, the modulator negates or neutralizes the activity of the virulence factor or a portion thereof. For example, the modulator binds a region of the virulence factor. In various embodiments, the region of the virulence factor is a conserved domain. Alternatively, the region of the virulence factor is a polymorphic domain.

In related embodiments of the composition, the modulator includes a nucleic acid binding protein that inhibits a gene encoding the virulence factor. For example, the binding protein binds to a signal that allows for the gene to encode the virulence factor.

In various embodiments of the composition, the virulence factor protein is at least one selected from the group of: a transporter for example a mesenteric lymph node required transporter (MrtAB), a lipopolysaccharide synthetase, a pH6 antigen, an invasin, an Ail, a flagellin, an attachment and effacement regulator, a cytoskeletal protein for example RodZ, and a portion thereof.

The virulence factor in various embodiments of the composition is encoded by a nucleotide sequence of at least one gene selected from the group of: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK 2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof, and the nucleotide sequences are listed in Table 1 using abbreviations for nucleotide sequences that are listed in GenBank or other genome databases such as European Nucleotide Archive, European Bioinformatics Institute, or GenomeNet. The material in computer readable form ASCII text file (236,723 bytes) created Jun. 7, 2013 entitled “34724118_Sequence_Listing_{—}06072013”, containing sequence listings numbers 1-103, has been electronically filed herewith and is incorporated by reference herein in its entirety.

In various embodiments, the virulence factor includes genes/operon YPK_{—}3222-YPK_{—}3221 (SEQ ID NO: 26), or a portion thereof. In a related embodiment, the gene encoding the virulence factor is a homolog of the nucleotide sequence, i.e., is at least about 60% identical or similar, or about 70%, about 80% identical, about 90% identical, or about 95% identical. In various embodiments, the gene includes a polymorphism or polymorphic domain. For example, polymorphism or the polymorphic domain includes a modification such as a deletion, a substitution, or an addition. Alternatively, the gene includes a conserved domain.

In various embodiments of the composition, the polymorphism or polymorphic domain includes a conservative substitution in which at least one nucleic acid encoding an amino acid residue is replaced with a different nucleic acid, such that the different nucleic acid encodes a different amino acid residue having a similar side chain. Families of amino acid residues having similar side chains have been defined in the art. These families include for example amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The virulence factor in various embodiments of the composition includes an amino acid sequence selected from the group of: YPK_{—}3221 (SEQ ID NO: 58), YPK_{—}3222 (SEQ ID NO: 59), YPK_{—}1234 (SEQ ID NO: 60), YPK_{—}2423 (SEQ ID NO: 61), YPK_{—}1292 (SEQ ID NO: 62), YPK_{—}2066 (SEQ ID NO: 63), YPK_{—}3575 (SEQ ID NO: 64), YPK_{—}1713 (SEQ ID NO: 65), YPK_{—}2406 (SEQ ID NO: 66), YPK_{—}3656 (SEQ ID NO: 67), YPK_{—}0453 (SEQ ID NO: 68), YPK_{—}0688 (SEQ ID NO: 69), YPK_{—}2424 (SEQ ID NO: 70), YPK_{—}3600 (SEQ ID NO: 71), YPK_{—}2199 (SEQ ID NO: 72), YPK_{—}4078 (SEQ ID NO: 73), YPK_{—}0208 (SEQ ID NO: 74), YPK_{—}3222-YPK_{—}3221 (SEQ ID NO: 103), and a portion thereof, and the amino acid sequences are listed in Table 1. The amino acid sequences are shown in GenBank or other databases such as European Bioinformatics Institute or GenomeNet. For example, the virulence factor in various embodiments of the composition includes an amino acid sequence in Table 1, for example the amino acid sequence is selected the group consisting of: SEQ ID NO: 58, 59 and 103, and a portion thereof.

An aspect of the invention provides a method for formulating a composition for treating or preventing a disease associated with a Gram-negative bacterial strain, the method including: identifying a mutant of a bacterial pathogen by mutagenizing with a transposon and screening for presence and growth in vivo in an organ of an infected host; sequencing bacterial nucleic acid isolated from the organ using the transposon sequence as a primer; identifying mutated genes permitting bacterial viability, thereby identifying mutations in genes encoding a virulence factor; engineering a modulator, or nucleotide acid sequence encoding the modulator, that inhibits function or expression of the virulence factor, and formulating the composition that treats or prevents the disease. In certain embodiments of the method, mutagenizing with the transposon includes generating a transgenic host animal for example having within its genome one or more copies of a mariner transposon. Mariner is a transposon originally isolated from Drosophila mauritiana, and other invertebrate and vertebrate species. See Craig et al. U.S. Pat. No. 7,709,696 issued May 4, 2010 and Sang et al. international patent publication number WO/1999/09817 published Mar. 4, 1999, each of which is incorporated by reference herein in its entirety.

Transposons are natural genetic elements capable of jumping or transposing from one position to another within the genome of a species. Mobilization of a transposon is dependent on the expression of a transposase enzyme which binds to sequences flanking the transposon DNA leading to the excision of DNA from one position in the genome and reinsertion elsewhere in the genome. Insertion into a gene sequence will lead to a change in gene function which may, in turn, result in a measurable phenotypic change in the whole organism.

The nucleotide sequence encoding the modulator is in various embodiments of the method encoded by at least one gene shown in Table 1 and is selected from the group of: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8). YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), YPK_{—}3222-YPK_{—}3221 (SEQ ID NO: 26), and a portion thereof, such that the nucleotide sequences are listed in GenBank or other genome databases such as European Nucleotide Archive, European Bioinformatics Institute, or GenomeNet. In a related embodiment, the nucleotide sequence is a homolog of the nucleotide sequences listed in Table 1. For example, the homolog has about 60%, 70%, 80%, or 90% similarity to the nucleotide sequences listed in Table 1, or other tables listed herein showing sequences for virulence factors.

The virulence factor in various embodiments of the method includes an amino acid sequence selected from the group of: YPK_{—}3221 (SEQ ID NO: 58), YPK_{—}3222 (SEQ ID NO: 59), YPK_{—}1234 (SEQ ID NO: 60), YPK_{—}2423 (SEQ ID NO: 61), YPK_{—}1292 (SEQ ID NO: 62), YPK_{—}2066 (SEQ ID NO: 63), YPK_{—}3575 (SEQ ID NO: 64), YPK_{—}1713 (SEQ ID NO: 65), YPK_{—}2406 (SEQ ID NO: 66), YPK_{—}3656 (SEQ ID NO: 67), YPK_{—}0453 (SEQ ID NO: 68), YPK_{—}0688 (SEQ ID NO: 69), YPK_{—}2424 (SEQ ID NO: 70), YPK_{—}3600 (SEQ ID NO: 71), YPK_{—}2199 (SEQ ID NO: 72), YPK_{—}4078 (SEQ ID NO: 73), YPK_{—}0208 (SEQ ID NO: 74), YPK_{—}3222-YPK_{—}3221 (SEQ ID NO: 103), and a portion thereof, and the amino acid sequences are listed in Table 1 and are found in in GenBank or other genome/protein databases such as GenomeNet. For example, the virulence factor in various embodiments of the method includes an amino acid sequence selected from the group of SEQ ID NOs: 58, 59 and 103, and a portion thereof.

An aspect of the invention provides a method for treating or preventing a disease associated with a Gram-negative bacterial strain in a subject, the method including; contacting a tissue of the subject with a composition containing a modulator of a virulence factor identified by mutagenizing cells of the strain and isolating mutated virulence factors, such that the modulator is specific to bind to the virulence factor to inhibit function or to bind to a gene encoding expression of the virulence factor, such that the composition prevents bacterial cell growth and infection of the strain in the tissue of the subject.

In various embodiments of the method, contacting the cells or the tissue of the subject with the modulator involves delivering a nucleic acid vector that inhibits expression of the virulence factor. For example the virulence factor includes a protein selected from the group of a transporter for example a mesenteric lymph node required transporter (MrtAB), a lipopolysaccharide synthetase, a pH6 antigen, an invasin, an Ail, a flagellin, and a cytoskeletal protein for example RodZ.

In related embodiments of the method, the gene encoding the virulence factor includes a nucleic acid vector including DNA or RNA, for example the nucleic acid vector includes a genetically engineered genome derived from at least one virus selected from the group of: adenovirus, adeno-associated virus, herpesvirus, and lentivirus.

Prior to contacting, the method in various embodiments involves engineering the modulator by constructing at least one of: mRNA, tRNA, rRNA, siRNA, RNAi, miRNA, and dsRNA, or a portion thereof. For example, constructing the siRNA involves using an expression cassette and/or recombinantly engineering a nucleic acid vector carrying the siRNA. In a related embodiment the method involves contacting donor cells and thereby transducing the donor cells with the nucleic acid vector. For example, the donor cells are stem cells or bacterial cells.

In an alternative embodiment the method involves prior to contacting the tissue, administering to donor cells a vector encoding the modulator, and thereby transducing the donor cells with the vector to produce the modulator. The term “transducing” is used to indicate that an infection, for example a viral infection or bacterial infection, results in transmission of genetic material that alters the genome of the recipient cells (e.g., donor cells, and cells in the tissue of the subject), providing genetic information to these cells that remains for a period of time. For example, the period of time is longer than a mere transient transfection. In a related embodiment contacting the tissue of the subject with the compositions involves transferring the transduced donor cells to the subject. For example, the donor cells are from a graft or a donor material. In various embodiments, the donor cells are autologous cells or heterologous cells. In various embodiments, the donor cells and the cells of the subject are matched for Human Leukocyte Antigens (HLA; also known as Major Histocompatibility (MHC) antigens).

In various embodiments of the method, the cells of the tissue or the donor cells include living cells. In various embodiments of the method, the cells of the tissue or the donor cells include at least one cell type selected from the group consisting of: epithelial, hematopoietic, stem, satellite, spleen, kidney, pancreatic, liver, neuronal, bone cells, muscle, adipotic, cartilage, glial, smooth or striated muscle, sperm, heart, lung, ocular, bone marrow, fetal cord blood, progenitor, tumor, peripheral blood mononuclear, leukocyte, and lymphocyte.

The method in a related embodiment further includes administering the modulator with a pharmaceutically acceptable salt and/or a pharmaceutically acceptable emollient. In a related embodiment, prior to contacting the tissue, the method involves formulating the composition in a sufficiently pure form to administer to a human, a pet, farm animal (e.g., cow and a pig), or a high value animal. For example contacting the tissue is performed by at least one route selected from the group of: parenteral, intravenous, intramuscular, intraperitoneal, intrapulmonary, intravaginal, rectal, oral, topical, sublingual, intranasal, ocular, transdermal, and subcutaneous.

In various embodiments of the method, contacting involves administering the modulator to the tissue selected from: muscular, epithelial, endothelial, lymph, and vascular. For example the tissue is at least one type selected from: eye, heart, kidney, thyroid, brain, stomach, gastrointestinal tract, abdomen, lung, liver, pancreas, stomach, liver, spleen, pancreas, and gall bladder.

The modulator in a related embodiment of the method is at least one selected from the group of: an antibody, an enzyme, a nucleic acid binding protein, and a fusion protein. For example, the modulator is a monoclonal antibody or humanized antibody specific for a portion of the virulence factor. For example the antibody is specific for a MrtAB heterodimeric protein or a portion thereof.

Contacting the cells in a related embodiment of the method involves contacting the tissue in situ or in vivo, for example the cells are at least one selected from the group consisting of: muscular, epithelial, endothelial, vascular, eye, heart, kidney, thyroid, brain, abdomen, stomach, gastrointestinal tract, lung, liver, pancreas, spleen, and lymph node. For example contacting the cells involves using a medical device such as a catheter, needle, patch, pump, bottle, nozzle, a bottle, a sprayer, a fluid dropper, a solution dropper, an inhaler, a gauze, a strip, a brush, or a syringe. In a related embodiment of the method, contacting the tissue involves adding the modulator to the tissue ex vivo to form a mixture, and then administering the mixture to the subject. For example, the method further involves prior to administering the mixture, determining efficacy of the modulator. In various embodiments, the efficacy is determined using an in vitro assay. Alternatively, the efficacy is determining using an in vivo assay and procedure/method described herein.

In various embodiments of the method, the nucleic acid vector encoding the modulator inhibits the virulence factor encoded by at least one gene shown in Table 1 and is selected from the group of: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10). YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof, such that the nucleotide sequence is listed for example in GenBank or GenomeNet. In a related embodiment, the modulator is a fusion protein that inhibits a plurality of the sequences in Table 1, for example at least one of which is YPK_{—}3221 (SEQ ID NO: 1) and YPK_{—}3222 (SEQ ID NO: 2). In various embodiments, the nucleic acid vector encoding the modulator inhibits the virulence factor encoded by genes/operon YPK_{—}3222-YPK_{—}3221 (SEQ ID NO: 26), or a portion thereof.

The virulence factor in various embodiments of the method includes an amino acid sequence selected from the group of: YPK_{—}3221 (SEQ ID NO: 58), YPK_{—}3222 (SEQ ID NO: 59), YPK_{—}1234 (SEQ ID NO: 60), YPK_{—}2423 (SEQ ID NO: 61), YPK_{—}1292 (SEQ ID NO: 62), YPK_{—}2066 (SEQ ID NO: 63), YPK_{—}3575 (SEQ ID NO: 64), YPK_{—}1713 (SEQ ID NO: 65), YPK_{—}2406 (SEQ ID NO: 66), YPK_{—}3656 (SEQ ID NO: 67), YPK_{—}0453 (SEQ ID NO: 68), YPK_{—}0688 (SEQ ID NO: 69), YPK_{—}2424 (SEQ ID NO: 70), YPK_{—}3600 (SEQ ID NO: 71), YPK_{—}2199 (SEQ ID NO: 72), YPK_{—}4078 (SEQ ID NO: 73), YPK_{—}0208 (SEQ ID NO: 74), YPK_{—}3222-YPK_{—}3221 (SEQ ID NO: 103), and a portion thereof, and the amino acid sequences are listed in Table 1, and the amino acid sequence is listed in GenBank or other genome databases such as GenomeNet. In various embodiments of the method, the virulence factor includes an amino acid sequence including SEQ ID NO: 58, SEQ ID NO: 59, SEQ ID NO: 103, and a portion thereof.

In various embodiments, the modulator produces a mutation in a cell of a subject. In embodiments of the method, the mutation includes at least one nucleotide change in the mrtAB gene. For example, the mutation is selected from the group of a substitution, a deletion, an addition. In certain embodiments, the cell or a tissue including the cell is selected from a type selected from: vascular, epithelial, endothelial, dermal, dental, connective, muscular, neuronal, facial, cranial, soft tissue, cartilage and collagen, brain, bone, bone marrow, joint tissue, and articular joints.

In various embodiments, the gene includes a conserved region. Alternatively, the at least one gene includes a polymorphism or polymorphic region. For example, the polymorphism or the polymorphic domain/region includes a conservative modification, e.g., a deletion, a substitution, or an addition.

In another embodiment of the method, the method involves, prior to contacting, engineering a vector including a nucleic acid sequence encoding a negative modulator of the at least one gene. For example, the at least one gene includes a conserved target. Alternatively, the at least one gene include a polymorphic target. The method involving engineering includes identifying a target domain of the gene or a protein or peptide encoded by the gene; locating a suitable nucleotide sequence of an encoding RNA for silencing by siRNA or antisense RNA; and constructing an siRNA or antisense expression cassette and inserting it into a recombinantly engineered nucleic acid of the vector. In various embodiments, the siRNA negatively modulates a mrtAB operon or a portion, for example the siRNA negatively modulates expression of a YPK_{—}3221 gene (SEC) ID NO: 1) and/or a YPK_{—}3222 gene (SEQ ID NO: 2).

An aspect of the invention provides a method of identifying a therapeutic agent for treating or preventing a disease associated with a Gram-negative bacterial strain in a subject, the method including: contacting a first sample of cells or tissue with the strain expressing a virulence factor, contacting a second sample of the cells or tissue with the strain and the therapeutic agent, and contacting a third sample of the cells or tissue with the strain and a control agent encoding a detectable protein that does not induce the colonization of the cells or the tissue, such that the first sample, second sample, and third sample are each from the subject; and measuring an amount of the marker in the first sample, the second sample, and the third sample, such that the marker is characteristic of the disease, so that the increased amount of the marker in the first sample compared to the second sample is a measure of treatment and protection by the therapeutic agent, so that a decreased amount of the marker in the third sample compared to the first sample is an indication that the agent is therapeutic, thereby identifying the potential therapeutic agent for treating or preventing the disease.

In various embodiments of the method, the Gram-negative bacterial strain is a short facultatively aerobic or micro-aerobic rod or an enteric strain. For example the strain includes at least one pathogen selected from the group of: Salmonella, Escherichia, Yersinia, Klebsiella, Shigella, Enterobacter, Serratia, Pseudomonas, and Citrobacter. In a related embodiment the strain is an Enterobacteriaceae. For example, the Gram-negative bacterial strain includes Y. pseudotuberculosis, Y. pestis, or Y. enterocolitica.

In various embodiments of the method, the detectable protein is at least one selected from the group of: a purification tag, a fluorescent protein, an enzyme, a colorimetric molecule for example a chemifluorescent protein. For example the detectable protein includes a green fluorescent protein.

The therapeutic agent in various embodiments of the method is selected from the group of: a vector for example a viral vector, a nucleic acid for example a DNA or RNA, a protein for example an enzyme or antibody, a small molecule, a carbohydrate for example a sugar. For example, the viral vector or a promoter that provides expression of the vector is derived from a mammalian subject such as a human, a mouse, or a pig. In an embodiment, the viral vector is derived from an avian species such as a chicken.

In a related embodiment of the method, measuring the marker involves detecting presence, activity or amount of a protein or a nucleic acid. In various embodiments, the marker is an indicator of a bacterial disease existing in at least one of: a lymphocyte, lymph node, skin, eye, mouth, brain, esophagus, breast, lung, liver, pancreas, spleen, bone, stomach, gastrointestinal tract, colon, kidney, and bladder. In various embodiments, the marker is an indicator of a bacterial disease existing in at least one mesenteric lymph node. For example, the marker is an indicator of bacterial shedding, or an immune response such as an immunoglobulin. In a related embodiment of the method, measuring further includes observing at least one of: cellular morphology, cell viability, cellular pathology, and tissue pathology.

In a related embodiment of the method, contacting the first sample, second sample and third sample is performed in an animal model in vivo, or is performed in an in vitro model, such that the cells or the tissue include at least one type selected from: muscular, epithelial, endothelial, vascular, eye, heart, kidney, thyroid, brain, abdomen, stomach, gastrointestinal tract, lung, liver, pancreas, spleen, and lymph node. For example, contacting the cells or tissue involves administering the modulator to a stratified organ.

An aspect of the invention provides a kit for modulating growth or severity of a disease associated with a Gram-negative bacterial strain, the kit including: a modulator of a virulence factor expressed by the Gram-negative bacteria, such that the virulence factor includes a protein, such that the modulator is specific to bind to the virulence factor to inhibit function or to bind to a gene encoding expression of the virulence factor, so that the composition prevents bacterial cell growth and infection of the strain in tissues of the subject; instructions for use; and, a container.

A related embodiment of the kit for any of pharmaceutical compositions and methods includes instructions for using the composition described herein. For example, the instructions for use include a method of identifying a therapeutic agent for treating or preventing a disease associated with the Gram-negative bacterial strain in a subject.

In a related embodiment of the kit, the gene includes a nucleic acid vector including DNA or a RNA for example mRNA, tRNA, rRNA, siRNA, RNAi, miRNA, and dsRNA, or a portion thereof. For example, the siRNA negatively modulates at least one gene selected from: a mrtAB, a mrtA, or a mrtB. For example the siRNA negatively modulates at least one gene selected from the group of SEQ ID NOs: 1-17, SEQ ID NO: 26, and a portion thereof.

The modulator in various embodiments of the kit contains at least one protein selected from the group of: an antibody, an enzyme, a fusion protein, and a nucleic acid binding protein. For example, the modulator is a monoclonal antibody specific for a surface protein or efflux pump. In various embodiments, the modulator is a monoclonal antibody specific for a peptide or protein encoded by at least one gene selected from Tables 1-6. For example the peptide or protein is encodes by at least one gene selected from SEQ ID NOs: 1-17, SEQ ID NO: 26, and a portion thereof.

The gene encoding the virulence factor is found in Table 1 in various embodiments of the kit, for example the gene is at least one selected from: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof, such that the nucleotide sequence is listed in GenBank. In various embodiments, the virulence factor is encoded by operon YPK_{—}3222-YPK_{—}3221 (SEQ ID NO: 26), or a portion thereof.

The virulence factor in various embodiments of the kit includes an amino acid sequence selected from the group of: YPK_{—}3221 (SEQ ID NO: 58), YPK_{—}3222 (SEQ ID NO: 59), YPK_{—}1234 (SEQ ID NO: 60), YPK_{—}2423 (SEQ ID NO: 61), YPK_{—}1292 (SEQ ID NO: 62), YPK_{—}2066 (SEQ ID NO: 63), YPK_{—}3575 (SEQ ID NO: 64), YPK_{—}1713 (SEQ ID NO: 65), YPK_{—}2406 (SEQ ID NO: 66), YPK_{—}3656 (SEQ ID NO: 67), YPK_{—}0453 (SEQ ID NO: 68), YPK_{—}0688 (SEQ ID NO: 69), YPK_{—}2424 (SEQ ID NO: 70), YPK_{—}3600 (SEQ ID NO: 71), YPK_{—}2199 (SEQ ID NO: 72), YPK_{—}4078 (SEQ ID NO: 73), YPK_{—}0208 (SEQ ID NO: 74), YPK_{—}3222-YPK_{—}3221 (SEQ ID NO: 103), and a portion thereof, and the amino acid sequences are listed in Table 1. In various embodiments of the method, the virulence factor includes an amino acid sequence including SEQ ID NOs: 58, 59, 103, and a portion thereof.

In various embodiments, the Gram-negative bacterial strain is a short facultatively aerobic or micro-aerobic rod, or an enteric strain. In various embodiments, the strain is at least one pathogen selected from the group of: Salmonella, Escherichia, Yersinia, Klebsiella, Shigella, Enterobacter, Serratia, Pseudomonas, and Citrobacter. For example, the pathogen is selected from: Yersinia pseudotuberculosis, Yersinia pestis, and Yersinia enterocolitica.

The virulence factor in various embodiments of the kit is at least one protein selected from the group of: a transporter for example a mesenteric lymph node required transporter (MrtAB), a lipopolysaccharide synthetase, a pH6 antigen, an invasin, an Ail, a flagellin, an attachment and effacement regulator, and a cytoskeletal protein for example RodZ. For example the modulator inhibits a transcription factor that induces expression of the protein.

An aspect of the invention provides a peptide encoded by a gene including any of the nucleotide sequences identified herein in Table 1 and as listed in Genbank, or a related molecule that encodes a peptide having at least 50% amino acid sequence similarity or identity at the peptide level in a Gram-negative bacterium, or a functional fragment thereof, for therapeutic or diagnostic use. In related embodiments, the peptide described herein has a nucleotide sequence similarity or an identity that is at least about: 60%, 70%, 80%, or 90%. In many embodiments, the sequence similarity or identity is at least 90% and for example includes conservative modifications in amino acid sequence of the encoded peptides.

In a related embodiment, the peptide further includes an amino acid sequence identified herein, for example the amino acid sequence is encoded by the nucleotide sequences described herein such as those shown in Table 1 or a homolog thereof.

An aspect of the invention provides a polynucleotide encoding a peptide described herein, for therapeutic or diagnostic use. For example the therapeutic or diagnostic use is for a human subject, or alternatively for a non-human subject such as for a horse, a pig, a cow, a goat, a dog, and a cat.

An aspect of the invention provides a recombinant host cell genetically modified to express a peptide described herein, for purposes of expression of a therapeutic protein. The protein can be administered as a therapeutic agent to the subject, or the subject can be administered the vector encoding the protein. In various embodiments, the host is selected from any of a variety of commercially available expression vector/host systems. For example, the host and/or host system includes microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems.

An aspect of the invention provides an attenuated microorganism including a mutation that disrupts expression of the nucleotide sequence described herein or identified herein or a homolog thereof. For example, the mutation is an addition or a substitution. In an alternative embodiment, the mutation is an insertional inactivation or a gene deletion. In various embodiments, the microorganism is Y. pseudotuberculosis, Y. enterocolitica, or Y. pestis.

In a related embodiment, the microorganism further includes a second mutation in a second nucleotide sequence. In a related embodiment, the microorganism further includes an antigenic target which is a heterologous antigen, or is a therapeutic peptide or a nucleic acid encoding the therapeutic peptide. The target which is a heterologous antigen for example is from an infectious organism selected from: a bacterium, a fungus, a virus, a protozoan, or a protein product thereof. In a related embodiment, the infectious organism is at least one Gram-positive bacterial strain or species selected from: a Bacillus, a Clostridium, a Staphylococcus, a Streptococcus, and an Enterococcus.

In various embodiments, the attenuated microorganism described herein is for therapeutic or diagnostic use. For example, the therapeutic use is treatment or prevention of a disease associated with an infectious organism e.g., a Gram-negative bacterial strain.

An aspect of the invention provides a vaccine having a peptide with an amino acid sequence described herein. For example the peptide is encoded by a gene including any of the nucleotide sequences identified herein in Tables 1-6 or a homolog or derivative thereof. For example, the peptide includes an amino acid sequence shown in Table 1.

An aspect of the invention provides a vaccine containing an attenuated microorganism described herein. An aspect of the invention provides an antibody that specifically binds to a peptide identified herein as associated with virulence of a microbial pathogen. For example, the antibody is a monoclonal antibody or a polyclonal antibody. In a related embodiment, the antibody is a humanized antibody.

An aspect of the invention provides a product for manufacture of a medicament for use in the treatment or prevention of a condition associated with infection by a Gram-negative bacterial strain or species, for example Yersinia. In various embodiments, the product is at least one selected from the modulator, the peptide, and the microorganism described herein. For example, condition is tuberculosis. In a related embodiment, the use is for veterinary treatment or for a non-human treatment.

Various embodiments provide the use of a peptide, polynucleotide or microorganism described herein for a screening assay for the identification of an antimicrobial drug, therapeutic agent, or vaccine.

An aspect of the invention provides a pharmaceutical composition for treating or preventing a disease associated with a Gram-negative bacterial strain, such that the composition specifically binds to a protein virulence factor to inhibit its function or to a gene encoding expression of the virulence factor to inhibit its expression, and prevents the Gram-negative bacterial cell growth and infection in tissues of the subject.

### BRIEF DESCRIPTION OF THE DRAWINGS

FIG. 1 panels A and B are a set of graphs showing that strains of plasmid deficient Yersinia pseudotuberculosis, Yptb(P⁻), bacteria colonized, grew and persisted in mouse deep tissue sites with little clonal loss.

FIG. 1 panel A is a graph that shows colony forming units (CFU), ordinate, in the spleen (diamond) and liver (square) as a function of time (days, abscissa) for C57BL/6 mouse subjects intravenously injected with a Yptb(P⁻) bacterial strain. An amount (1×10⁵) of Yptb(P⁻) strain was intravenously injected to the subjects, and organs were collected three days post-infection. Bacterial numbers in the organs were quantified by assaying the total CFU per organ. N=three to six mice, mean CFU is plotted ±standard deviation.

FIG. 1 panel B is a graph showing average number of unique transposon insertion clones in the Input library and observed in each organ, ordinate, in the spleen and liver of C57BL/6 mice intravenously injected with Yptb(P⁻) bacteria six days earlier (abscissa). The symbol +/− indicates the standard deviation and the N value is three mice. HPI indicates hours post inoculation and DPI indicates days post-inoculation.

FIG. 2 panels A, B and C are a drawing and graphs showing genetic screening for chromosomal Yptb virulence factors in two control libraries. The screen was performed by plating an overnight culture of each of two individual libraries of 10,000 transposon mutants (more than 200,000 colonies), which were used to generate genomic DNA. This is defined as an Input sample. For each library, there were at least two Input samples, with a corresponding set of Output samples, defined as the colonies from an individual infected liver derived from a given Input injection dose.

FIG. 2 panel A is a drawing showing the number of genes that were mutated in each input library, and the number of genes mutated in both libraries. Library #1 (left most circle) contained unique 618 clones, library #2 (right most circle) contained 493 unique clones, and libraries #1 and #2 shared 1977 unique clones (middle circle). The total number of clones obtained was 3088.

FIG. 2 panel B is a graph showing binary logarithm (log2) of the ratio of each genus of biological replicates (BR) 1 and BR2 (ordinate) as a function of the gene number of the Input Library #2 of Yptb sequenced separately (abscissa). In FIG. 2 panel B a dashed line represents one standard deviation, and a solid line represents two standard deviations.

FIG. 2 panel C is a histogram of 1977 genes mutated in both library #1 and library #2 as shown in FIG. 2 panel A. FIG. 2 panel C is a graph of number of genes that have a threshold value (ordinate) as a function of log₂ of average ratio of liver Output/Input (abscissa). The X-axis extends to include values for all genes.

FIG. 3 panels A-D are a set of graphs showing that mrtAB gene, an operon encoding an ABC family transporter, was required for growth of Yptb (P⁻) bacteria in liver and spleen of subjects injected with the bacteria. Statistical significance (*) was determined by nonparametric Mann-Whitney test.

FIG. 3 panel A is a graph showing CFU observed in each organ (ordinate) in the liver and the spleen of mice three days after intravenous injection of Yptb (P−) cells or mrtAB-deficient Yptb cells (Yptb(P−)ΔmrtAB). Mice (N=four to six mice) were injected/inoculated intravenously (1×10⁵), and three days later organs were collected, and bacterial number was determined by CFU observed in each organ: circle indicates spleen for subject injected with Yptb (P−) bacteria; downward triangle indicates spleen for subject injected with mrtAB-deficient Yptb (P⁻) bacteria; square indicates liver for subject injected with Yptb (P−) bacteria; and upward triangle indicates liver for subject injected with mrtAB-deficient Yptb (P⁻) bacteria. Data show greater number of CFU in the spleen than for the liver for each bacterial strain. Subjects injected with Yptb (P−) bacteria having a frame deletion of mrtAB were observed to have a two-log or three-log fold decrease in CFU in the liver and spleen, respectively, compared to subjects injected with Yptb (P−) bacteria. In FIG. 3 panel A the median CFU is presented by horizontal rectangle/line.

FIG. 3 panel B is a graph showing absorbance at a wavelength of 600 nm (OD600), ordinate, as a function of time (abscissa) for cultures that contained either Yptb (P−) bacteria or mrtAB-deficient Yptb (ΔmrtAB) bacteria. Each bacterial culture was grown at 37° in 2XYT broth culture. Data show similar growth at 37° in 2XYT broth culture for Yptb (P−) cells or mrtAB-deficient Yptb (ΔmrtAB) cells. Thus deletion of mrtAB operon in the Yptb bacteria did not alter growth at 37° in 2XYT broth culture compared to the Yptb (P−) bacteria. Data are mean of three replicates, and error bars (±) correspond to the standard deviation.

FIG. 3 panel C is a graph showing a graph showing CFU observed in each organ (ordinate) in the spleen of mice three days after being intravenously injected with an amount of: a Yptb(P−) bacteria with an empty vector (circle), a mrtAB-deficient Yptb (Yptb (P−) ΔmrtAB) bacteria with an empty vector as control (triangle), or Yptb(P−) ΔmrtAB bacteria and carries an in trans complementation plasmid encoding mrtAB, pmrtAB (square). Subjects (N=5 mice) were injected intravenously (1×10⁵ cells) with bacteria, and spleens were collected three days post-infection and analyzed as in FIG. 3 panel A. Data show subjects injected with the Yptb(P⁻) ΔmrtAB bacteria had a two-fold and three-fold lower number of CFU in the spleen than subjects injected with the Yptb (P−) bacteria. Subjects injected with the Yptb(P⁻) ΔmrtAB bacteria carrying a complementation plasmid encoding mrtAB (pmrtAB) were observed to have CFU in the spleen comparable to the subjects injected with the Yptb(P−) bacteria, and much a greater CFU in the spleen than subjects Yptb(P⁻) ΔmrtAB bacteria. Data show that the complementation plasmid encoding mrtAB (pmrtAB) reversed the effect observed for Yptb(P⁻) ΔmrtAB bacteria, and the mrtAB encoding plasmid was observed to produce growth of Yptb (P⁻) bacteria in the spleen as well as the liver, resulting from rescue of Yptb(P⁻) ΔmrtAB bacteria in trans with plasmid carrying mrtAB. Median value is indicated by a horizontal rectangle.

FIG. 3 panel D is a graph showing CFU observed (ordinate) in the liver and the spleen of as a function of time (hours; abscissa) for mice intravenously injected (1×10⁵) with an either a Yptb (P−) bacteria or a mrtAB-deficient Yptb (ΔmrtAB) bacteria. Each strain was grown at 37° in 2XYT broth culture prior to injection into the subjects. Subjects (N=three mice) were then sacrificed and organs were collected between four hours and three days post-injection, and bacterial number was determined by quantifying CFU observed in each organ: circle indicates spleen for subject injected with Yptb (P−) bacteria; square indicates liver for subject injected with Yptb (P−) bacteria; downward triangle indicates liver for subject injected with Yptb (P−) bacteria; and upward triangle indicates spleen for subject injected with mrtAB-deficient Yptb bacteria. Data show a greater number of CFU in the spleen and the liver for subjects injected Yptb (P−) bacteria compared to subjects injected with Yptb (P⁻) ΔmrtAB bacteria. Subjects administered the Yptb (P⁻) ΔmrtAB mutant strains yielded an increased CFU in the spleen compared to the liver.

FIG. 4 panels A-D are a set of graphs showing that in wildtype Yptb strains the mrtAB operon is specifically required to colonize the mesenteric lymph node in subjects. Median values for the CFU on the graphs are indicated by a horizontal rectangle. Statistical significance (*) was determined by nonparametric Mann-Whitney test.

FIG. 4 panel A is a graph showing CFU observed (ordinate) in the liver and the spleen of mice three days after intravenously administration of a mrtAB-deficient Yptb (Yptb(P+)ΔmrtAB) mutant cells, or wildtype Yptb(P+) cells (abscissa). Subjects (N=four to six mice) were inoculated intravenously with 10³ of the strains, organs were collected three days post-inoculation, and CFU determined in organs: circle indicates spleen for subject injected with wildtype bacteria; downward triangle indicates spleen for subject injected with mrtAB-deficient Yptb bacteria; square indicates liver for subject injected with wildtype bacteria; and upward triangle indicates liver for subject injected with mrtAB-deficient Yptb bacteria. Comparable CFU values were found in the spleen and liver of subjects intravenously injected with Yptb(P+)ΔmrtAB bacteria or with wildtype Yptb(P+) bacteria.

FIG. 4 panel B is a graph showing CFU observed (ordinate) in the small intestine, Peyer's patches, and the mesenteric lymph node of mice one day after oral administration of mrtAB-deficient Yptb(P+) mutant bacteria or a wildtype Yptb(P+) bacteria. Mice (ten) were orally inoculated with 2×10⁹ bacteria and organs were collected one day later: circle indicates small intestine of subject administered with wildtype bacteria; x indicates small intestine of subject administered mrtAB-deficient Yptb bacteria; square indicates Peyer's patches of subject administered wildtype bacteria; diamond indicates Peyer's patches of subject administered mrtAB-deficient Yptb bacteria; upward triangle indicates mesenteric lymph node of subject administered wildtype bacteria; and downward triangle indicates mesenteric lymph node of subject administered mrtAB-deficient Yptb bacteria. The dashed line indicates the limit of detection. Similar CFU values were observed for liver and spleen in subjects orally administered the wildtype Yptb(P⁺) strain or the Yptb(P⁺)ΔmrtAB strain. A reduced CFU was observed in the mesenteric lymph node of subjects orally administered the Yptb(P⁺)ΔmrtAB bacteria compared to the wildtype bacteria. Yptb (P⁺) bacteria required the mrtAB operon for optimal colonization of mesenteric lymph nodes, as data show at least a two-fold lower CFU in nodes from subjects orally administered the mrtAB-deficient Yptb(P+) bacteria compared to nodes in subjects orally administered wildtype bacteria. Symbols used: SL indicates small intestine, PP indicates Peyer's patches, and MLN indicates mesenteric lymph node.

FIG. 4 panel C is a graph showing CFU observed in each (ordinate) mesenteric lymph node of mice one day after orally being administered either mrtAB-deficient Yptb(P+) bacteria carrying empty vector (upward triangle), or a Yptb(P+) ΔmrtAB bacteria carrying a complementation plasmid encoding MrtAB protein (diamond). Control subjects were orally administered wildtype Yptb(P+) bacteria (circle). Subjects (N=eight or nine mice) were orally administered (2×10⁹) bacteria, and organs were collected one day post-infection and analyzed for number CFU: Data show similar CFU values in the mesenteric lymph nodes for subjects orally administered the mrtAB-deficient Yptb bacteria having the complementation plasmid encoding mrtAB compared to control subjects orally administered wildtype bacteria. Subjects orally administered the mrtAB-deficient Yptb bacteria had at least a fold lower CFU value in the mesenteric lymph nodes compared to subjects orally administered the mrtAB-deficient Yptb bacteria carrying the complementation plasmid encoding MrtAB protein, and also compared to control subjects orally administered wildtype Yptb(P+) bacteria. Data show that a defect in mesenteric lymph node colonization was caused by deletion/absence of mrtAB operon in the Yptb mutant cells, and that the defect in colonization was reversed/rescued in trans by a plasmid encoding the mrtAB protein.

FIG. 4 panel D is a graph showing on the ordinate CFU observed in spleen or mesenteric lymph node of mice one day after intraperitoneal injection of mrtAB-deficient Yptb(P+) bacteria, or wildtype Yptb(P+) bacteria. Intraperitoneal injection contained 2×10⁵ bacteria. Organs were collected from the subjects (four mice) one day after injection, and were analyzed for CFU: circle indicates spleen of subject injected with wildtype bacteria; square indicates spleen of subject injected with mrtAB-deficient Yptb bacteria; diamond indicates mesenteric lymph nodes of subject injected with wildtype bacteria; and triangle indicates liver of subject injected with mrtAB-deficient Yptb bacteria. Data show similar CFU values in the spleen of subjects intraperitoneally injected with each of mrtAB-deficient Yptb(P+) bacteria and wildtype bacteria. Subjects intraperitoneally injected with mrtAB-deficient Yptb bacteria were observed to have lower CFU values in the mesenteric lymph nodes compared to control subjects injected with wildtype bacteria. The defect in mesenteric lymph node colonization by mrtAB-deficient Yptb(P+) bacteria was caused by the deletion/absence of the gene encoding the MrtAB heterodimeric protein.

FIG. 5 panels A and B are a graph and immunoblots showing that ATPase activity of MrtB protein is required for optimal growth in vivo.

FIG. 5 panel A is a graph showing CFU observed in each spleen (ordinate) for mice intravenously injected (1×10⁵) three days earlier with bacterial cells: circle indicates Yptb(P⁻), square indicates Yptb(P⁻)ΔmrtAB, triangle indicates Yptb(P⁻)ΔmrtAB/pmrtA⁺mrtB⁺-flag complementation vector, and diamond indicates Yptb(P⁻)ΔmrtAB/pmrtA⁺mrtB*-flag complementation vector with K380A (*) mutation in MrtB protein. N=six or eight mice. The Yptb(P⁻) bacteria and the FLAG-tagged MrtB bacteria carrying the plasmid encoding mrtAB (pmrtA⁺B⁺-flag) was observed to have increased growth in the spleen compared to the Yptb(P⁻) ΔmrtAB bacteria and the Yptb(P⁻)ΔmrtAB/pmrtA⁺mrtB*-flag bacteria carrying complementation vector with K380A (*) mutation in MrtB protein. *P: Statistical significance was determined by nonparametric Mann-Whitney test.

FIG. 5 panel B is a set of photographs of Western blots of bacteria grown in vitro to examine the effect of disrupting the MrtB-FLAG Walker A box on MrtB-FLAG expression. Gels lanes were loaded with the following bacteria grown in LB in vitro at 26° or 37° with: Yptb(P⁻) ΔmrtAB/vector (Lane 1, 26° C., Lane 2, 37° C.), Yptb(P⁻)ΔmrtAB/pmrtA⁺mrtB⁺-flag (Lane 3, 26° C., Lane 4, 37° C.), or Yptb(P⁻)ΔmrtAB/pmrtA⁺mrtB*-flag (*K380A) (Lane 6, 26° C., Lane 7, 37° C.). Blots were stripped and were probed for an antibody specific with MrtB Walker A box protein (FIG. 5 panel B top row). Blots were re-probed with anti-S2 antibody for a loading control (FIG. 5 panel B top row). Data show expression of the MrtB Walker A box protein in the cultures containing the plasmid encoding MrtB protein.

FIG. 6 panels A-E are a drawing, a graph and a set of photomicrographs showing construction and characterization of a YopE reporter strain.

FIG. 6 panel A is a drawing of a yopE reporter construction (yopE-STOP::FLAG-mCherry). A FLAG-mCherry sequence was inserted immediately after the yopE stop codon to serve as a reporter for yopE expression. The Ysc type III secretion system allows Yersinia to translocate virulence proteins such as YopE into the cytosol of eukaryotic cells. The YopE effectors possess an individual chaperone called a SycE protein, viz., encoded by sycE gene.

FIG. 6 panel B is a set of photomicrographs showing that expression by the yopE reporter gene is properly regulated. Wildtype bacteria and krF-deficient bacteria carrying yopE-STOP::FLAG-mCherry construct were grown at 37° C. (FIG. 6 panel B first row and second row respectively). The thermoregulatory protein LcrF is a transcriptional activator of the thermally regulated virulent yopE gene. Bacteria carrying yopE-STOP::FLAG-mCherry construct was grown at 26° C. (FIG. 6 panel B third row). FIG. 6 panel B shows visualization of the photomicrographs by phase contrast (left column) and fluorescence microscopy (right column). Data show FLAG-mCherry was not detectibly expressed at 37° C. in the bacterial culture containing the lcrF-deficient bacteria carrying the yopE-STOP::FLAG-mCherry gene.

FIG. 6 panel C is a set of immunoblots showing that reporter expression does not affect endogenous yopE expression. Each of strains wild-type (wt; first column), replicates of wildtype yopE-STOP::FLAG-mCherry (wt::FLAG-mcherry #1 and #2; second and third columns from the left), and an lcrF-deficient yopE-STOP::FLAG-mCherry (ΔlcrF::FLAG-mCherry; fourth column) were grown in yop inducing conditions. Bacterial lysates were loaded in gels and analyzed by Western blotting using antibodies specific for YopE protein (FIG. 6 panel C top row) and FLAG epitopic tag (FIG. 6 panel C bottom row). Data show YopE protein expression in the lysates from the wild-type (wt; first column), and in lysates from the yopE-STOP::FLAG-mCherry (second and third columns). No expression of YopE protein was detected in lysates of lcrF-deficient yopE-STOP::FLAG-mCherry cells (fourth column).

FIG. 6 panel D is a set of photomicrographs showing that reporter strain fluorescence decreases in the absence of yop expression. Cells carrying yopE-STOP::FLAG-mCherry were grown in yop-inducing conditions (37° C. and low presence of calcium) from two hours, were washed and were shifted to non-inducing conditions (26° C. and high presence of calcium). Samples were taken every two hours and were imaged by phase contrast (left column) and fluorescence microscopy (right column). Data show strong mCherry expression in yopE-STOP::FLAG-mCherry cells at zero and two hours, and decreased expression at four hours compared to two hours. Little or no expression at six hours and eight hours.

FIG. 6 panel E is a graph of mean mCherry fluorescence (left ordinate) quantified on a per-bacterium basis for yopE-STOP::FLAG-mCherry cells, and absorbance measured at a wavelength of 600 nm (OD; right ordinate) as a function of time (hours/abscissa). Data show strong fluorescence at about two hours followed by a decrease in fluorescence, and an increase in absorbance increased as a function of time during the entire period.

FIG. 7 panels A-D are a set of photomicrographs and a graph showing that Yptb(P⁺)-GFP/YopE-mCherry bacteria expressed YopE reporter protein in the spleen and the mesenteric lymph nodes (MLN), and that the nodes also contained neutrophils. Representative images show distinct staining in the micro-colonies in the spleen (FIG. 7 panel A) or MLN (FIG. 7 panel B) of subjects injected Yptb(P⁺)-GFP/YopE-mCherry bacteria. The amount of Yptb(P⁺)-GFP/YopE-mCherry bacteria or Yptb(P⁺)-GFP bacteria intravenously administered (10³; FIG. 7 panel A and D) and, orally administered to subjects (2×10⁹; FIG. 7 panels B and E) was chosen to approximately synchronize the presence of the bacterial infections in the spleen and the mesenteric lymph nodes, respectively, as during oral infection the spleen is colonized later than the mesenteric lymph nodes. The representative images show distinct staining of micro-colonies in the spleen (FIG. 7 panel A) and MLN (FIG. 7 panel B) of subjects injected or orally administered Yptb(P⁺)-GFP/YopE-mCherry bacteria.

FIG. 7 panels A is a set of representative photomicrographs of mesenteric lymph nodes from mice that were intravenously injected (10³ cells) with Yptb(P⁺)-GFP/YopE-mCherry bacteria two days earlier. Left column: tissue visualized for mCherry fluorescence; middle column: tissue visualized for GFP; right column: merges of the mCherry fluorescence and GFP photomicrographs of the first and second columns with Hoechst dye staining.

FIG. 7 panels B is a set of representative photomicrographs of the spleen of mice orally administered 2×10⁹ Yptb(P⁺)-GFP/YopE-mCherry cells two days earlier. Organs were collected two days post-oral administration and analyzed. Left column: tissue visualized for mCherry fluorescence; middle column: tissue visualized for GFP; right column: merges of the mCherry fluorescence and GFP photomicrographs of the first and second columns with Hoechst dye staining.

FIG. 7 panel C is a graph showing the ratio of median mCherry fluorescence intensity and GFP intensity, ordinate, in the in the spleens and mesenteric lymph nodes (abscissa) of subjects orally administered Yptb(P⁺)/GFP/YopE-mCherry bacteria, or Yptb(P⁺)/GFP bacteria. N=three mice (GFP control) or N=seven mice for Yptb(P⁺)/GFP/YopE-mCherry in MLN, and N=eight mice for Yptb(P⁺)/GFP/YopE-mCherry in spleen.

FIG. 7 panels D is a set of representative photomicrographs of spleens from mice intravenously injected with 10³ cells two days earlier with Yptb(P⁺)-GFP or wildtype Yptb bacteria only. Left column: tissue visualized for presence of Yptb bacteria; middle column: tissue visualized for presence of neutrophils using anti-Ly6G clone 1A8 (Ly6G) antibody; right column: merges of the first and second columns with Hoechst dye staining.

FIG. 7 panel E is a set of photomicrographs of mesenteric lymph nodes from mice orally administered (2×10⁹ cells) two days earlier with Yptb(P⁺)-GFP bacteria, or wildtype bacteria only. Left column: tissue visualized for presence of Yptb bacteria; middle column: tissue visualized for presence of neutrophils using anti-Ly6G clone 1A8 (Ly6G) antibody; right column: merges of the first and second columns with Hoechst dye staining.

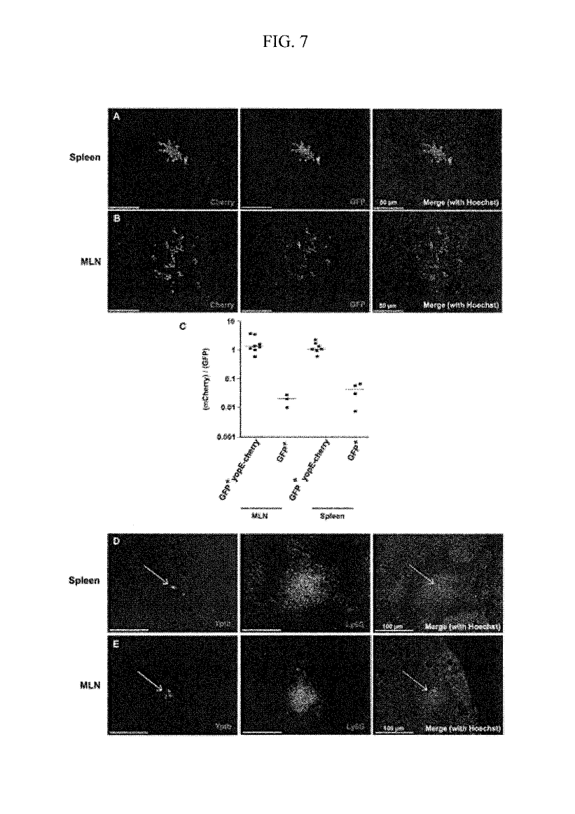

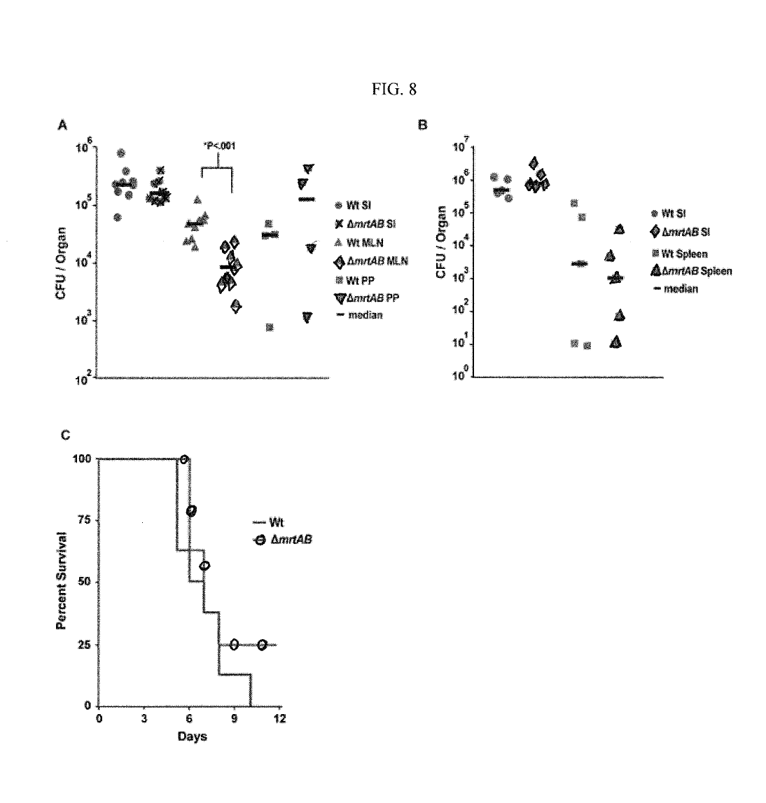

For both FIG. 7 panels D and E, two days after inoculation the subjects were sacrificed and tissue sections visualized for presence of Yptb bacteria, were stained for neutrophils using monoclonal anti-Ly6G clone 1A8 (Ly6G) antibody, and were stained for DNA using Hoechst dyes. The 1A8 monoclonal antibody reacts with Ly-6G, a 21-25 kilodalton glycophosphatidylinositol (GPI)-anchored protein, which together with the structurally related Ly-6C protein comprises the granulocyte receptor-1 antigen (Gr-1). Gr-1 is expressed on monocytes, neutrophils and subsets of macrophages, plasmacytoid dendritic cells and T cells.

FIG. 8 is a set of graphs showing reduced growth of the Yptb(P+) mrtAB mutant bacteria in the mesenteric lymph nodes compared to wildtype Yptb(P+). The Yptb(P+) mrtAB mutant spleen colonization ability and lethality in mice were comparable to the wildtype. The median value in the graphs is indicated by a horizontal rectangle.

FIG. 8 panel A is a graph showing CFU observed in each organ (ordinate) in small intestine, Peyer's patches, and the MLN of mice four days after oral administration of mrtAB-deficient Yptb(P+) bacteria or wildtype bacteria. Mice were orally inoculated with 2×10⁹ of either Yptb(P⁺) cells or Yptb(P⁺)ΔmrtAB, organs were collected after four days, and the CFU in each organ was determined: circle, small intestine administered wildtype; x, small intestine administered mrtAB-deficient Yptb; upward triangle, MLN administered wildtype; diamond, MLN administered mrtAB-deficient Yptb; square, Peyer's patches administered wildtype; and downward triangle, Peyer's patches administered mrtAB-deficient Yptb. Data show that subjects orally administered mrtAB deficient Yptb(P+) bacteria exhibited a modest decrease in CFU and colonization of the MLN four days post-infection compared to subjects orally administered wildtype bacteria. N=four mice (PP) or nine mice (SI and MLN). *P: Statistical significance was determined by nonparametric Mann-Whitney test.

FIG. 8 panel B is a graph showing CFU (ordinate) in the small intestine and the spleen of mice two days after oral administration of either mrtAB-deficient Yptb(P+) or wildtype Yptb(P+). Mice were orally administered 10⁹ cells, organs were collected two days later, and analyzed for CFU: circle indicates small intestine of subject administered wildtype bacteria; diamond indicates small intestine of subject administered mrtAB-deficient Yptb bacteria; square indicates spleen of subject administered wildtype bacteria; and triangle indicates spleen of subject administered mrtAB-deficient Yptb bacteria. Data show that Yptb(P+) and Yptb(P⁺)ΔmrtAB display comparable early colonization of the spleen following oral infection. N=5 mice.

FIG. 8 panel C is a Meyer-Kaplan survival plot showing percent (%) survival days after oral administration of 10⁹ mrtAB-deficient Yptb(P+) bacteria (circle), or control administered wildtype Yptb(P+) (line). Data show that after eight days a greater percentage of subjects orally administered Yptb (P⁺)ΔmrtAB Yptb(P⁺)-GFP bacteria survived longer than control subjects oally administered wildtype bacteria. N=8 mice.

### DETAILED DESCRIPTION

Yersinia pseudotuberculosis, Yersinia pestis, and Yersinia enterocolitica are three mammalian pathogens in the Yersinia genus. Yersinia pestis, the causative agent of plague, has had a profound effects on human civilization, killing one of three people in Europe during the Black Death (Wren, B. W. 2003 Nat Rev Microbiol 1: 55-64). In contrast, the highly related Y. pseudotuberculosis (Yptb) and Y. enterocolitica (Ye) usually cause self-limiting gastrointestinal infections. The three Yersinia species share a tropism for growth in lymph nodes. Infection with Y. pestis results in dramatically inflamed lymph nodes, and Yptb or Ye bacterial infections are associated with acute mesenteric lymphadenitis due to colonization of the mesenteric lymph nodes (Smego. R. A. et al. 1999 Eur J Clin Microbiol Infect Dis 18: 1-15). The pathogenic Yersinia bacteria also share a conserved virulence plasmid, which encodes a Type III Secretion System, TTSS, and its associated translocated substrate proteins, called Yops (Cornelis, G. R. et al. 1998. Microbiol Mol Biol Rev 62: 1315-1352).

The virulence plasmid is required for optimal Yptb bacterial growth in a variety of mammalian organs, including the small intestine, cecum, Peyer's patches, liver, spleen, and lung (Une, T. et al. 1984 Infect Immun 43: 895-900; Balada-Llasat, J. M. et al. 2006 PLoS Pathog 2: e86). Detailed study of the components of the virulence plasmid, including the TTSS, Yops, and the adhesin YadA, revealed that each of these proteins is required during growth in these organs (El Tahir, Y. et al. 2001 Int J Med Microbiol 291: 209-218; Logsdon, L. K. et al. 2003 Infect Immun 71: 4595-460; Fisher, M. L. et al. 2007 Infect Immun 75: 429-442). The Yops protein in particular may be required to disarm many components of the host innate immune response, with potential functions including interfering with phagocytosis and misregulating immune signaling pathways (El Tahir, Y. et al. 2001 Int J Med Microbiol 291: 209-218; Trosky, J. E. et al. 2008 Cell Microbiol 10: 557-565). However, the loss of the virulence plasmid and its arsenal of encoded Yops did not reduce the growth of Yptb bacteria in the mesenteric lymph nodes, as measured by colony forming units (Balada-Llasat, J. M. et al. 2006 PLoS Pathog 2: e86). The mesenteric lymph nodes, therefore, behave anomalously in that chromosomally encoded Yptb virulence factors appear to be sufficient for growth in this organ.

Several genetic screens have been performed using pathogenic Yersinia bacterial strains to determine virulence factors required during animal infection (Mecsas, J. et al. 2001 Infect Immun 69: 2779-2787; Darwin, A. J. et al. 1999 Mol Microbiol 32: 51-62; Karlyshev, A. V. et al. 2001 Infect Immun 69: 7810-7819; Flashner, Y. et al. 2004 Infect Immun 72: 908-915). A number of chromosomally-encoded factors are required for efficient systemic disease or during intestinal colonization, including invasin, the two component regulatory system (PhoP/PhoQ). Ybt, and pH 6 antigen (Cathelyn, J. S. et al. 2006 Proc Natl Acad Sci USA 103: 13514-13519; Isberg, R. R. et al. 1987 Cell 50: 769-778; Rakin, A. et al. 1999 Infect Immun 67: 5265-5274; Oyston, P. C. 2000 Infect Immun 68: 3419-3425). However, previous genetic screens analyzed only a limited number of genes. In addition, no systematic identification of proteins encoded by the chromosome, in the absence of contributions from the virulence plasmid, has been performed. Thus, multiple chromosomal Yptb virulence factors remain to be discovered and analyzed.

A highly conserved virulence plasmid encoding a type III secretion system is shared by the three Yersinia species most pathogenic for mammals. Although factors encoded on this plasmid enhance the ability of Yersinia to thrive in their mammalian hosts, the loss of this virulence plasmid does not eliminate growth or survival in host organs. Yields of viable plasmid-deficient Yersinia pseudotuberculosis, Yptb, are indistinguishable from wildtype Yptb bacteria within mesenteric lymph nodes.

To identify chromosomal virulence factors that allow for plasmid-independent survival during systemic infection of mice, the methods in the Examples herein were used to generate transposon insertions in plasmid-deficient Yptb bacteria, and screen a library having more than 20,000 sequence-identified insertions. Previously uncharacterized loci were identified, including insertions in mrtAB gene, an operon encoding an ABC family transporter. The mrtAB operon was observed to have the most profound phenotype in a plasmid-deficient background. The absence of MrtAB protein expression, however, had no effect on growth in the liver and spleen of a wild type bacteria having an intact virulence plasmid. Most important, the absence, manipulation, or deletion of the mrtAB operon in Yptb bacteria was observed to cause a severe defect in colonization of the mesenteric lymph nodes. Although this decreased colonization in the mesenteric lymph nodes might indicate a lack of expression of the type III secretion system by wildtype Yptb bacteria in the mesenteric lymph nodes, the presence a reporter for YopE shows that expression of the system was robust.

Data from Examples herein demonstrate that the ATPase activity of MrtAB protein was required for growth in mice, indicating that transport activity was required for virulence. Without being limited by any particular theory or mechanism of action, it is here envisioned that MrtAB protein functions as an efflux pump that exports material across the inner membrane, as the ATPase activity was found to have enhanced resistance to ethidium bromide and increased sensitivity to pyocyanin. A number of candidate virulence factors were identified by screening greater than 20,000 plasmid-deficient sequence-identified transposon insertion mutants obtained from infection of mice. The mrtAB operon, a previously uncharacterized heterodimeric ABC transporter that is critical for the growth and persistence of plasmid-cured Yptb bacteria in mice was identified. Examples herein show that mrtAB operon and MrtAB transporter was necessary for wildtype Yptb (P⁺) cells to colonize only a single organ, the mesenteric lymph nodes.

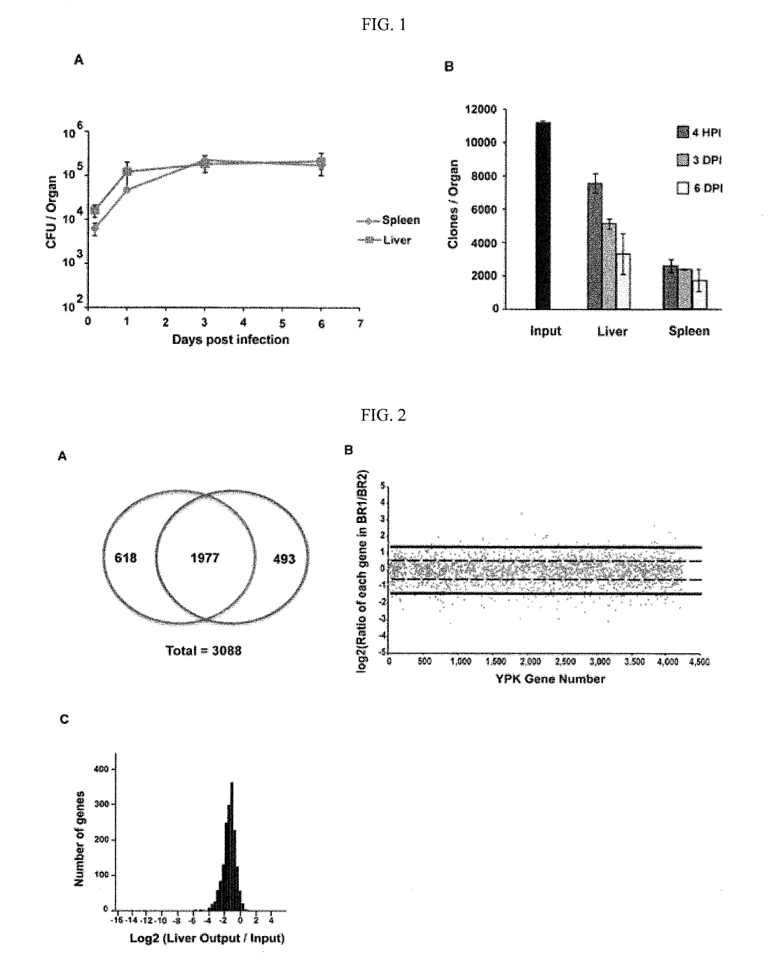

The importance of the virulence plasmid for the growth and spread of Yptb bacteria in various organs has been documented using a variety of inoculation routes (tine, T. et al. 1984 Infect Immun 43: 895-900; Balada-Llasat. J. M. et al. 2006 PLoS Pathog 2: e86). The absence of the plasmid has been reported to have an inconsequential effect on growth in the mesenteric lymph nodes in spite of the large number of known virulence factors encoded by plasmid (Balada-Llasat, J. M. et al. 2006 PLoS Pathog 2: e86). Data herein show that plasmid-deficient Yptb cells grew more than twenty-fold in both the liver and spleen and persisted for longer than a week at high levels in these organ sites (FIG. 1). This surprising ability of the plasmid-deficient Yptb bacteria to persist in the face of an antagonistic immune system indicated that there is a range of unidentified Yptb chromosomal virulence factors. In methods shown in the Examples herein, more than 20,000 transposon insertion mutants were screened for ability to grow and to persist for days in vivo, and a number of putative chromosomal virulence factors were identified (FIG. 2 and Table 1). A larger number of Yersinia mutants were screened than in the published combined accounts of in vivo genetic screens performed with Yersinia pseudotuberculosis, Yersinia pestis, and Yersinia enterocolitica, (Mecsas. J. et al. 2001 Infect Immun 69: 2779-2787; Darwin, A. J. et al. 1999 Mol Microbiol 32: 51-62; Karlyshev, A. V. et al. 2001 Infect Immun 69: 7810-7819; Flashner, Y. et al. 2004 Infect Immun 72: 908-915).

Five virulence factors (Darwin, A. J. et al. 1999 Mol Microbiol 32: 51-62; Oyston, P. C. et al. 2000 Infect Immun 68: 3419-3425; and Makoveichuk, E. 2003 J Lipid Res 44: 320-330) previously identified in the transposon mutant insertion screen served as indicator of the effectiveness of both the strategy that used herein and in use of Yptb(P⁻) bacteria as the genetic background for the screening. While it is envisioned that some of the Yptb genes identified herein will be required only in the P background, the five known virulence factors identified in methods used in examples herein are evidence that many genes in Table 1 are required also in the wild type background. Without being limited by any particular theory or mechanism of action, it is here envisioned that most of the 11 genes that encode proteins involved in amino acid or purine synthesis are likely to be required in the wild type strain harboring the virulence plasmid. For example, gene aroA was shown to be essential for growth of Yersinia enterocolitica in mice (Bowe, F. et al. 1989 Infect Immun 57: 3234-3236), and both gene aroA and purine synthesis genes are essential for Salmonella typhimurim pathogenesis (O'Callaghan, D. et al. 1988 Infect Immun 56: 419-423).

Mutants defective in amino acid and purine synthesis have been used to generate candidate vaccine strains for a variety of bacterial pathogens (O'Callaghan, D. et al. 1988 Infect Immun 56: 419-423; Bowe, F. et al. 1989 Infect Immun 57: 3234-3236; Jackson, M. et al. 1999 Infect Immun 67: 2867-2873; Simmons, C. P. 1997 Infect Immun 65: 3048-3056). Without being limited by any particular theory or mechanism of action, it is here envisioned that genes identified herein provide additional platforms for vaccine development.

A significant result from data in the Examples herein was identification of novel Yptb chromosomal virulence factors. Table 1 describes 18 candidate chromosomal virulence factors, none of which were previously investigated in Yptb, and many of which have not been investigated in any pathogen. One of the few characterized virulence factors in this list is gene apaH, which is required for both invasion and adherence of Salmonella enterica to mammalian cells (Ismail, T. M. et al. 2003 J Biol Chem 278: 32602-32607). Two other characterized genes (Tables 3, 4 and 5) are flagellar regulon members flgD and flgC. In Mycobacterium tuberculosis, OppD was recently shown to reduce both apoptosis and inflammatory cytokine release from macrophages, which could have parallels for Yptb bacteria evading immune detection (Dasgupta, A. et al. 2010 PloS One 5: e12225). RodZ, a structural protein required for maintaining normal bacterial morphology, is a regulator of post-transcriptional processing in Shigella sonnei (Mitobe, J. et al. 2011 EMBO Reports 12: 911-916).

The data obtained herein identified 14 genes predicted to be involved in LPS modification (Table 1). Two genes encoding essential steps of the O antigen (O-Ag) synthesis pathway are identified in Table 1; gene wecA (YPK_{—}4033; SEQ ID NO: 43) is involved in initiating synthesis of the O subunit, and waaL (YPK_{—}3646; SEQ ID NO: 42) encodes the ligase that attaches O-Ag to the lipid A core outer saccharide (Marolda, C. L. et al. 2004 Microbiology 150: 4095-4105). Surprisingly, mutations in either of these genes rendered Yptb cells unable to grow at elevated temperatures (Table 1). The O-Ag synthesis operon encodes proteins that produce the nucleotide-diphospho (NDP) sugars subunits of O-antigen, as well as the O-Ag polymerase, flippase, and chain length regulator (Skurnik, M. et al. 2003 Carbohydr Res 338: 2521-2529; Kalynych, S. et al. 2011 J Bacterial 193: 3710-3721). An important observation from mutation data herein is that some products are required for growth 37° C. while, in general, most products are required for growth of Yptb(P⁻) in deep tissue sites. YPK_{—}3177 (SEQ ID NO: 55) corresponds to wzz gene, the predicted O-Antigen chain length regulator. Interestingly the wzz gene was observed not to be essential for growth in the mouse infection model used herein, as transposon insertions in this gene located at the end of the operon had no effect on growth in the liver (Table 3 found in supplemental data in Crimmins, G. T. et al. 2012PLoS Pathog 8(8): e1002828, and in appendices in U.S. provisional application Ser. No. 61/656,640 filed Jun. 7, 2012, each of which is incorporated by reference herein in its entirety). RfaH gene, YPK_{—}3937 (SEQ ID NO: 44) in Table 1, is included in the O-Ag group because it is a bacterial elongation factor that is required for the expression of several genes including genes of O-Ag operon.

A number of the same members of the homologous O-Ag synthesis operon were identified during screening for Y. enterocolitica virulence factors (Darwin, A. J. et al. 1999 Mol Microbial 32: 51-62), indicating that O-Ag production is necessary also in the presence of the virulence plasmid. O-Ag plays a pivotal role in the pathogenesis of Y. pseudotuberculosis and other Gram negative bacterial pathogens. Detailed analysis of O-Ag status of Y. enterocolitica shows that O-Ag production is critical for virulence, perhaps due to its role in the expression of other virulence factors, such as invasin and Ail (Bengoechea J. A. et al. 2004 Mol Microbial 52: 451-469). Other data have implicated the O-Ag of S. enterica in resistance to bile salts and anti-microbial peptides (Kong, Q. et al. 2011 Infect Immun 79: 4227-39). Positions YPK_{—}1834-1835 (SEQ ID NO: 52 and SEQ ID NO: 56 respectively) are part of an operon predicted to play a role in adding amino sugars to lipid A, which has also been implicated in resistance to anti-microbial peptides (Marceau, M. et al. 2004 Microbiology 150: 3947-3957).

The nucleic acid sequence of operon mrtAB analyzed in Examples herein, as insertions in these gene resulted in two of the most significant growth deficits observed in the screen data. It was observed that operon mrtAB encodes a poorly characterized, hypothetical ABC— type transporter. The mrtAB operon (previously annotated as mdlAB for “multi-drug resistance like”) is highly conserved in most Enterobacteriaceae, with a predicted protein sequence similarity of 85% conserved for mrtA among E. coli, Shigella flexneri, S. enterica, and Klebsiella pneuomoniae. High levels of expression of mrtAB homologs in S. enterica correlated with increased resistance to a fluoroquinolone antibiotic, although deletion of these genes had no effect on fluoroquinolone resistance (Chen, S. et al. 2007 Antimicrob Agents Chemother 51: 535-542). The effect of mrtAB expression on resistance of E. coli to a variety of toxic compounds was analyzed, and no effect on drug resistance was observed (Nishino, K. et al. 2001 J Bacteriol 183: 5803-5812).

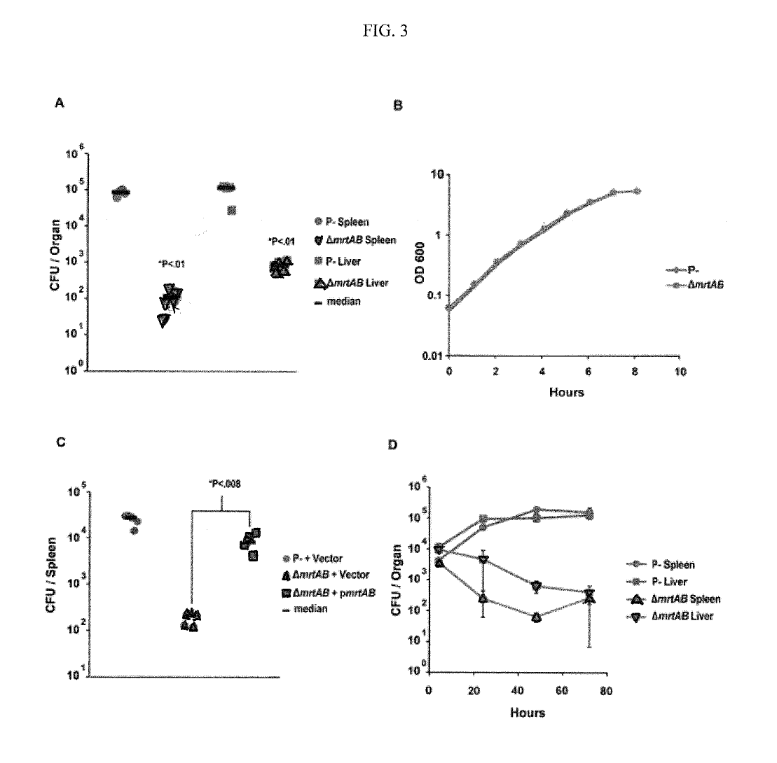

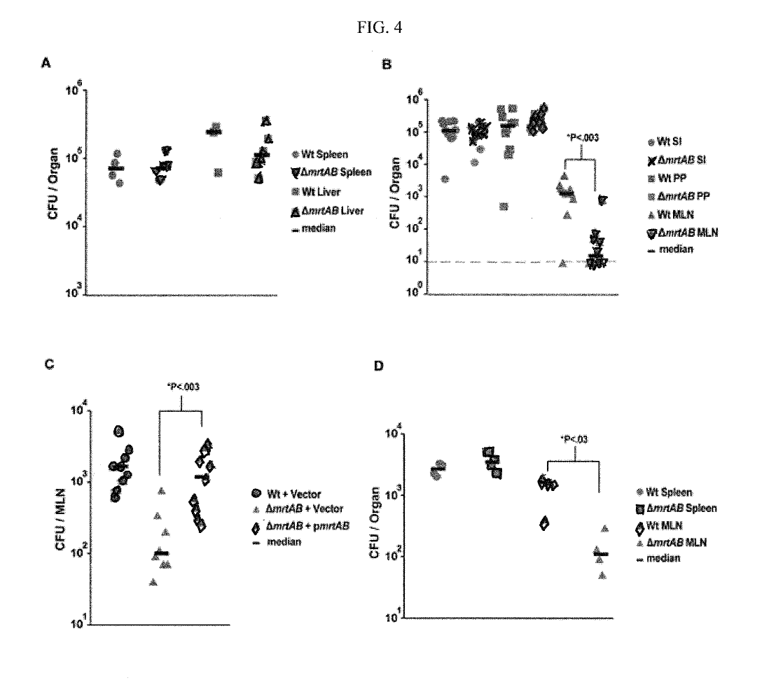

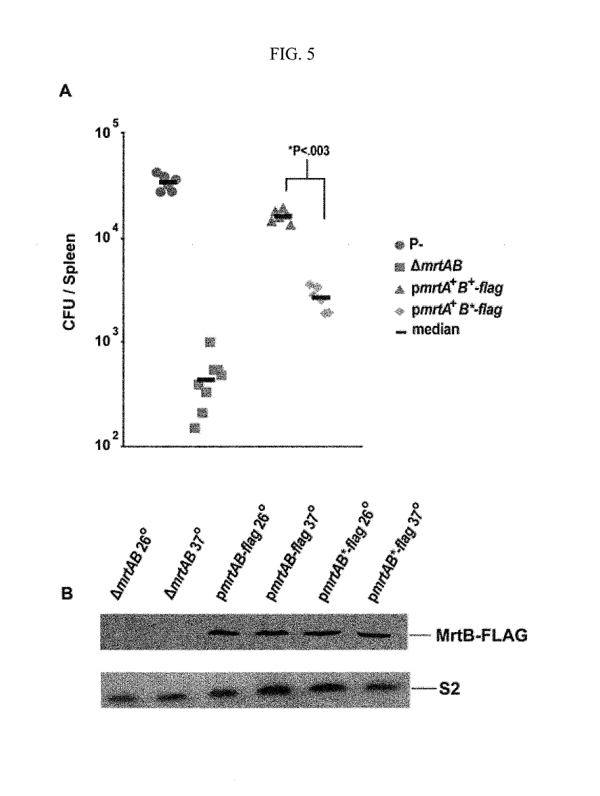

In-frame deletions of either the mrtAB operon or of the individual genes within the operon recapitulated the phenotypes from the screen, without any noticeable effect on growth in vitro (FIG. 2 and FIG. 3). Complementation in trans rescued the mrtAB deletion mutant and bacterial yields in the liver and spleen were observed to near the levels of Yptb (P⁻) bacteria (See FIG. 2). Surprisingly, the putative transporter was not required for growth of the fully virulent Yptb (P⁺) bacteria in the spleen and liver as well as the small intestine and Peyer's patches (FIG. 4). Further data showed that for bacteria in the P⁺ background, i.e., Yptb(P⁺), the mrtAB operon was required only for growth and colonization in the mesenteric lymph nodes (FIG. 4).

A number of studies have demonstrated that productive infection by Yptb bacteria requires the same set of virulence factors in a variety of organ sites, such as the Peyer's patches, spleen, liver and lung (Une, T. et al. 1984 Infect Immun 43: 895-900; Balada-Llasat, J. M. et al. 2006 PLoS Pathog 2: e86; Fisher, M. L. 2007 Infect Immun 75: 429-442). The Yptb infection of the mesenteric lymph nodes is the anomaly, in that it is the only organ in which the virulence plasmid is not required (Balada-Llasat, J. M. et al. 2006 PLoS Pathog 2: e86). That mrtAB genes are essential for infection of mesenteric lymph nodes provided additional evidence for the unique nature of the mesenteric lymph nodes interaction with Yptb cells. This raises the possibility that fully virulent Yptb bacteria persisted in an entirely different selective environment in the mesenteric lymph nodes (MLN) than in other organ sites. As Yptb bacteria interacts with and preferentially translocate Yops into neutrophils in vivo, whether altered neutrophil co-localization with bacteria in the spleen relative to MLN was tested in Examples herein. Data show that neutrophils similarly surrounded the bacterial microcolonies in almost all bacterial foci in the MLN or in the spleen (FIG. 7). In addition, it was observed that the virulence plasmid was capable of rescuing/increasing growth of an mrtAB mutant bacteria in every organ except the mesenteric lymph nodes (FIG. 3 and FIG. 4), and that the virulence plasmid was dispensable for growth only in the MLN (Balada-Llasat, J. M. et al. 2006 PLoS Pathog 2: e86). The possibility existed that the plasmid-encoded type III secretion system substrates are not expressed by Yptb bacteria in the MLN. However, no difference in YopE expression was detected in the spleen and MLN of subjects orally administered or intravenously injected with a bacteria carrying a YopE-mCherry reporter (FIG. 7 panels A and B).

Characterization of the wildtype Yptb(P+) infection of the spleen and MLN did not reveal any clear differences that could explain the differential requirement for MrtAB in the colonization of these two organs (FIG. 4 and FIG. 7). Therefore, to further analyze the role of MrtAB protein in the mesenteric lymph nodes, the analysis of subjects orally infected was extended to four days post-infection. Interestingly, the difference in bacterial burden in the MLN alter four days of infection was largely erased, with the mrtAB mutant displaying only a five-fold lower colonization of the mesenteric lymph nodes, compared to an approximately 100-fold lower burden at one day post-infection (FIG. 4 and FIG. 8). These data show that MrtAB protein was specifically required for initial mesenteric lymph node colonization, and that MrtAB transporter protein does not play a role in post-colonization growth in this organ. Without being limited by any particular theory or mechanism of action, it is here envisioned that with a much lower dose of infection, the mrtAB mutant gene could be completely deficient for MLN colonization throughout the infection.

To determine whether the transport activity of MrtAB transporter protein is required to support Yptb survival in mouse tissue sites, a Walker A box of MrtB peptide was mutated, and tested for the ability to rescue/modulate the growth of Yptb (P⁻) ΔmrtAB bacteria in the spleen (FIG. 7). Mutation of the MrtB Walker A box protein strongly reduced the growth of Yptb bacteria in mouse spleens, without noticeably altering the expression of the protein during growth in broth culture (FIG. 5 panels A and B). The data in Examples herein show that the ATPase transport activity of the MrtAB ABC transporter protein was an important and/or necessary for its role in promoting Yptb bacteria growth in vivo. The sequence and genetic organization of operon mrtAB is consistent with MrtAB protein forming a heterodimeric ABC family exporter. There exists a conserved TEVGERV motif in both MrtA and MrtB peptides that is found only in ABC export systems (Davidson, A. L. et al. 2008 Microbiol Mol Biol Rev 72: 317-364; Dawson, R. J. et al. 2006 Nature 443: 180-185).

Furthermore, over-expression of MrtAB protein enhanced resistance to ethidium bromide, an activity that was largely dependent on the transport activity of MrtB peptide (Table 2). Conversely, multi-copy expression of mrtAB operon resulted in increased susceptibility to pyocyanin, a phenotype that required the MrtB ATPase. Pyocyanin disrupts the cell membrane respiratory chain, although the mechanism of pyocyanin toxicity is unclear (Hassan, H. M. et al. 1980 J Bacteriol 141: 156-163; Baron, S. S. et al. 1981 Antimicrob Agents Chemother 20: 814-820; Gusarov, I. et al. 2009 Science 325: 1380-1384). Pyocyanin blocks transport that is dependent on the proton motive force, consistent with a disruption of respiration (Baron, S. S. et al. 1989 Curr Microbial 18: 223-230). Many of the components of the electron transport chain are accessible to or located within the periplasm. Therefore, expression of a transporter that moves pyocyanin into the periplasm, as a result of export across the inner membrane, could readily increase susceptibility to this toxic compound.

There are numerous potential roles that a bacterial transporter could play in virulence, including uptake of nutrients, resistance to toxic compounds, or secretion of an immunomodulatory bacterial compound. Data obtained from Examples herein show that MrtAB protein is involved in cell secretion, including that MrtAB protein is homologous to other ABC family exporters and that MrtAB provided resistance to pyocyanin, a toxic compound. Were MrtAB involved in secretion of a toxic host compound, it is unlikely to be a toxic compound encountered in the small intestine, liver, spleen, or Peyer's patches, as the ΔmrtAB mutant bacteria colonized these organs at comparable levels to wildtype Yptb bacteria (FIG. 4 panel B). While it is possible that MrtAB protein is required for resistance to an unknown toxic host compound that is unique to the mesenteric lymph nodes, it is unlikely because mrtAB-deficient Yptb bacteria were capable of colonizing the MLN at a level that is only moderately less than that of wildtype Yptb bacteria (FIG. 8 panel A), and mrtAB operon was required for Yptb(P−) to survive in the liver and spleen (FIG. 3).

To determine if MrtAB transporter protein was required for dissemination of Yptb(P+) bacteria from the intestine, the ability of the mrtAB-deficient bacteria to colonize the spleen following oral infection was analyzed in Examples herein. Interestingly, the mrtAB-deficient bacteria colonized and grew in the spleen at a level comparable to wildtype Yptb(P+), indicating that MrtAB transporter protein is specifically required for transit of bacteria to the MLN (FIG. 4 panel B and FIG. 8 panel B). While it is unknown how Yptb bacteria travel to different organs during oral infection, it has been observed that the mesenteric lymph nodes and the spleen are colonized independently, with the spleen being successfully colonized later during infection, following bacterial replication in the intestine, and the mesenteric lymph nodes being colonized earlier, within hours of infection (Balada-Llasat, J. M. et al. 2006 PLoS Pathog 2: e86; Barnes, P. D. et al. 2006 J Exp Med 203: 1591-1601). It is unknown how Yptb bacteria traffics to the MLN, though dendritic cells are thought to be important for Salmonella enterica serovar Typhimurium to gain access to this immune organ (Voedisch, S. et al. 2009 Infect Immun 77: 3170-3180). A possibility exists that MrtAB transporter protein is required to survive interaction with trafficking dendritic cells, either by exporting an immunomodulatory bacterial compound, or providing resistance to a toxic dendritic cell compound. Without being limited by any particular theory or mechanism of action, it is here envisioned that during transit with an innate immune cell to the mesenteric lymph nodes, Yptb bacteria refrain from using the TTSS in order to avoid disrupting the normal trafficking of the host cell. This possible characteristic of Yptb bacteria could explain why the virulence plasmid rescued an mrtAB-deficient mutant bacteria in all aspects of virulence except colonization of the mesenteric lymph nodes.

Pathogenic Yersinia species share a tropism for growth in lymph nodes, and lymph node pathology is commonly observed in infections with all Yersinia species, ranging from inflammation and swelling of regional lymph nodes (Y. pestis bubonic plague), to inflammation of the mesenteric lymph nodes (Y. enterocolitica and Y. pseudotuberculosis oral infections). See Smego. R. A. et al. 1999. Eur J Clin Microbial Infect Dis 18: 1-15. A high degree of conservation of MrtA and MrtB molecules was observed in Yersinia strains, as there is 99% identity in Y. pestis and 91-93% identity in Y. enterocolitica. Without being limited by any particular theory or mechanism of action, it is here envisioned that MrtAB protein plays a role in the colonization of lymph nodes by all pathogenic Yersinia species, including Y. pestis and Y. enterocolitica. The role of MrtAB in strains of Y. pseudotuberculosis that do not share the phoP mutation present in the YPIII strain used in this study was analyzed in Examples herein (Grabenstein, J. P. et al. 2004 Infect Immun 72: 4973-4984). Finally, MrtA and MrtB proteins are also highly conserved in other bacterial pathogens that colonize the MLN, including Salmonella enterica serovar Typhimurium (76-79% identity), allowing a possibility that transport mediated by the mrtAB operon/MrtAB protein may be a common mechanism by which bacterial pathogens colonize this immune organ.

Examples herein identified a number of candidate virulence factors in Y. pseudotuberculosis. MrtAB protein is the first mesenteric lymph node specific virulence factor identified in Yersinia species. Further analysis and examination of the ABC transporter MrtAB and its substrate(s) provides valuable insight into the interaction of Y. pseudotuberculosis with the mesenteric lymph nodes and its unique requirements for establishing bacterial replication in this site.

Compositions, methods and kits herein bind to virulence factors of Gram-negative bacteria using a modulator. As used herein, a “modulator” refers to any molecule, compound, or construct that modulates function and/or expression of Gram-negative virulence factors.

The modulator in various embodiments includes a protein, a nucleotide acid encoding a protein that modulates expression of the virulence factor, an agent that binds to the virulence factor, and a nucleotide sequence encoding expression of the agent. For example, the nucleotide sequence, which encodes a peptide or protein, is in various embodiments substantially identical to the genes identified in Table 1 herein. In various embodiments, the modulator or agent inhibits expression or activity of the virulence factor to reduce or eliminate the negative effects of the Gram-negative bacteria.

Modulators of virulence factor in examples herein include conservative sequence modifications to the nucleotide sequences or amino acid sequences described herein. As used herein, the term “conservative sequence modifications” refers to amino acid or nucleotide modifications that do not significantly affect or alter the characteristics of the modulator, for example by substitution of an amino acid with a functionally similar amino acid. Such conservative modifications include substitutions, additions and deletions. Modifications of amino acid sequences or nucleotide sequences is achieved using any known technique in the art e.g., site-directed mutagenesis or PCR based mutagenesis. Such techniques are described in Sambrook et al., Molecular Cloning: A Laboratory Manual, fourth edition, Cold Spring Harbor Press, Plainview, N.Y., 2012 and Ausubel et al., Current Protocols in Molecular Biology, fifth edition, John Wiley & Sons, New York, N.Y., 2002.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

In certain embodiments, the amino acid sequence or nucleotide sequence of the modulator is a sequence that is substantially identical to that of the wild type sequence, for example the sequences identified in Table 1 herein, e.g., SEQ ID NOs: 1-17 and SEQ ID NO: 26. The term “substantially identical” is used herein to refer to a first sequence that contains a sufficient or minimum number of residues that are identical to aligned residues in a second sequence such that the first and second sequences can have a common structural domain and/or common functional activity. For example, amino acid sequences that contain a common structural domain having at least about 60% identity, or at least 70%, 75%, 80%, 85%, 90%, 95%, or 99% identity. For example, the modulator has at least 60% identity, at least 65% identity, at least 70% identity, at least 75% identity, at least 80% identity, at least 85% identity, at least 90% identity, at least 95% identity, or at least 99% identity to the amino acid sequence of a wild-type bacterial sequence. In certain embodiments the nucleotide sequence of the modulator has at least about 60%, 70%, 75%, 80%, 85%, 90%, 95%, or 99% identity to a wild-type bacterial nucleotide sequence, e.g., mrtAB gene.

Calculations of sequence identity between sequences are performed as follows. To determine the percent identity of two amino acid sequences for example, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences are accomplished using a mathematical algorithm. Percent identity between two amino acid sequences is determined using an alignment software program using the default parameters. Suitable programs include, for example, CLUSTAL W by Thompson et al., Nuc. Acids Research 22:4673, 1994, BL2SEQ by Tatusova and Madden, FEMS Microbiol. Lett. 174:247, 1999, SAGA by Notredame and Higgins, Nuc. Acids Research 24:1515, 1996, and DIALIGN by Morgenstern et al., Bioinformatics 14:290, 1998.

### Vectors

In various embodiments of the invention herein, a method for modulating virulence factors of Gram-negative bacteria, for example Salmonella, Escherichia and Yersinia, is provided, the method including contacting cells or tissue with a pharmaceutical composition including a modulator, or a nucleotide sequence that is a source of expression the modulator. For example, the modulator is a recombinantly produced protein administered in situ or ex vivo. The term “recombinant” refers to proteins produced by manipulation of genetically modified organisms, for example micro-organisms or eukaryotic cells in culture.

In accordance with the present invention a source of the modulator includes polynucleotide sequences that encode the transcription factor, for example, engineered into recombinant DNA molecules to direct expression of the protein or a portion thereof in appropriate host cells. To express a biologically active protein, a nucleotide sequence encoding the protein, or functional equivalent, is inserted into an appropriate expression vector, i.e., a vector that contains the necessary nucleic acid encoding elements that regulate transcription and translation of the inserted coding sequence, operably linked to the nucleotide sequence encoding the amino acid sequence of the protein or portion thereof.

Methods that are well known to those skilled in the art are used to construct expression vectors containing a nucleic acid sequence encoding for example a protein or a peptide operably linked to appropriate transcriptional and translational control elements. These methods include in vitro recombinant DNA techniques, synthetic techniques and in vivo recombination or genetic recombination. Techniques are described in Sambrook et al., Molecular Cloning: A Laboratory Manual, fourth edition, Cold Spring Harbor Press, Plainview, N.Y., 2012.

A variety of commercially available expression vector/host systems are useful to contain and express a protein or peptide encoding sequence. These include but are not limited to microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. See Ausubel et al., Current Protocols in Molecular Biology, John Wiley & Sons, New York, N.Y., 2004.

Virus vectors include, but are not limited to, adenovirus vectors, lentivirus vectors, retrovirus vectors, adeno-associated virus (AAV) vectors, and helper-dependent adenovirus vectors. For example, the vectors deliver a nucleic acid sequence that encodes a modulator protein or agent that binds to a target or antigen that as shown herein modulates virulence or growth of Gram-negative bacterial cells. Adenovirus packaging vectors are commercially available from American Type Tissue Culture Collection (Manassas, Va.). Methods of constructing adenovirus vectors and using adenovirus vectors are shown in Klein et al., Ophthalmology, 114:253-262, 2007 and van Leeuwen et al., Eur. J. Epidemiol., 18:845-854, 2003.

Adenovirus vectors have been used in eukaryotic gene expression (Levrero et al., Gene, 101:195-202, 1991) and vaccine development (Graham et al., Methods in Molecular Biology: Gene Transfer and Expression Protocols 7, (Murray, Ed.), Humana Press, Clifton, N.J., 109-128, 1991). Further, recombinant adenovirus vectors are used for gene therapy (Wu et al., U.S. Pat. No. 7,235,391).

Recombinant adenovirus vectors are generated, for example, from homologous recombination between a shuttle vector and a provirus vector (Wu et al., U.S. Pat. No. 7,235,391). The adenovirus vectors herein are replication defective, for example, are conditionally defective, lacking adenovirus E1 region, and a polynucleotide encoding MrtAB or portion thereof is introduced at the position from which the E1-coding sequences have been removed. The polynucleotide encoding the mrtAB genes or portion thereof alternatively is inserted in the E3 region, or is inserted in an E4 region using a helper cell line.

Lentiviral vector packaging vectors are commercially available from Invitrogen Corporation (Carlsbad Calif.). An HIV-based packaging system for the production of lentiviral vectors is prepared using constructs in Naldini et al., Science 272: 263-267, 1996; Zufferey et al., Nature Biotechnol., 15: 871-875, 1997; and Dull et al. J. Virol. 72: 8463-8471, 1998.

A number of vector constructs are available to be packaged using a system, based on third-generation lentiviral SIN vector backbone (Dull et al., J. Virol. 72: 8463-8471, 1998). For example the vector construct pRRLsinCMVGFPpre contains a 5′ LTR in which the HIV promoter sequence has been replaced with that of Rous sarcoma virus (RSV), a self-inactivating 3′ LTR containing a deletion in the U3 promoter region, the HIV packaging signal, RRE sequences linked to a marker gene cassette consisting of the Aequora jellyfish green fluorescent protein (GFP) driven by the CMV promoter, and the woodchuck hepatitis virus PRE element, which appears to enhance nuclear export. The GFP marker gene allows quantitation of transfection or transduction efficiency by direct observation of UV fluorescence microscopy or flow cytometry (Kafri et al., Nature Genet., 17: 314-317, 1997 and Sakoda et al., J. Mol. Cell. Cardiol., 31: 2037-2047, 1999).

Manipulation of retroviral nucleic acids to construct a retroviral vector containing a gene that encodes a protein, and methods for packaging in cells are accomplished using techniques known in the art. See Ausubel, et al., 1992, Volume 1, Section 111 (units 9.10.1-9.14.3); Sambrook, et al., 1989. Molecular Cloning: A Laboratory Manual. Second Edition. Cold Spring Harbor Laboratory Press, Cold Spring Harbor, N.Y.; Miller, et al., Biotechniques. 7:981-990, 1989; Eglitis, et al., Biotechniques. 6:608-614, 1988; U.S. Pat. Nos. 4,650,764, 4,861,719, 4,980,289, 5,122,767, and 5,124,263; and PCT patent publications numbers WO 85/05629, WO 89/07150, WO 90/02797, WO 90/02806, WO 90/13641, WO 92/05266, WO 92/07943, WO 92/14829, and WO 93/14188, each of which is incorporated by reference herein in its entirety.

A retroviral vector is constructed and packaged into non-infectious transducing viral particles (virions) using an amphotropic packaging system. Examples of such packaging systems are found in, for example, Miller, et al., Mol. Cell. Biol. 6:2895-2902, 1986; Markowitz, et al., J. Virol. 62:1120-1124, 1988; Cosset, et al., J. Virol. 64:1070-1078, 1990; U.S. Pat. Nos. 4,650,764, 4,861,719, 4,980,289, 5,122,767, and 5,124,263, and PCT patent publications numbers WO 85/05629, WO 89/07150, WO 90/02797, WO 90/02806, WO 90/13641, WO 92/05266, WO 92/07943, WO 92/14829, and WO 93/14188.

Generation of “producer cells” is accomplished by introducing retroviral vectors into the packaging cells. Examples of such retroviral vectors are found in, for example, Korman, et al., Proc. Natl. Acad. Sci. USA. 84:2150-2154, 1987; Morgenstern, et al., Nucleic Acids Res. 18:3587-3596, 1990; U.S. Pat. Nos. 4,405,712, 4,980,289, and 5,112,767; and PCT patent publications numbers WO 85/05629, WO 90/02797, and WO 92/07943.

Herpesvirus packaging vectors are commercially available from Invitrogen Corporation, (Carlsbad, Calif.). Exemplary herpesviruses are an α-herpesvirus, such as Varicella-Zoster virus or pseudorabies virus; a herpes simplex virus such as HSV-1 or HSV-2; or a herpesvirus such as Epstein-Barr virus. A method for preparing empty herpesvirus particles that can be packaged with a desired nucleotide segment is shown in Fraefel et al. (U.S. Pat. No. 5,998,208, issued Dec. 7, 1999).

The herpesvirus DNA vector can be constructed using techniques familiar to the skilled artisan. For example, DNA segments encoding the entire genome of a herpesvirus is divided among a number of vectors capable of carrying large DNA segments, e.g., cosmids (Evans, et al., Gene 79, 9-20, 1989), yeast artificial chromosomes (YACS) (Sambrook, J. et al., Molecular Cloning: A Laboratory Manual, fourth edition, Cold Spring Harbor Press, Cold Spring Harbor, N.Y., 2012) or E. coli F element plasmids (O'Conner, et al., Science 244:1307-1313, 1989).

For example, sets of cosmids have been isolated which contain overlapping clones that represent the entire genomes of a variety of herpesviruses including Epstein-Barr virus, Varicella-Zoster virus, pseudorabies virus and HSV-1. See M. van Zijl et al., J. Virol. 62, 2191, 1988; Cohen, et al., Proc. Nat'l Acad. Sci. U.S.A. 90, 7376, 1993; Tomkinson, et al., J. Virol. 67, 7298, 1993; and Cunningham et al., Virology 197, 116, 1993.

AAV is a dependent parvovirus in that it depends on co-infection with another virus (either adenovirus or a member of the herpes virus family) to undergo a productive infection in cultured cells (Muzyczka, Curr Top Microbiol Immunol, 158:97 129, 1992). For example, recombinant AAV (rAAV) virus is made by co-transfecting a plasmid containing the gene of interest, for example, the NR×3.2 gene. Cells are also contacted or transfected with adenovirus or plasmids carrying the adenovirus genes required for AAV helper function. Recombinant AAV virus stocks made in such fashion include with adenovirus which must be physically separated from the recombinant AAV particles (for example, by cesium chloride density centrifugation).

Adeno-associated virus (AAV) packaging vectors are commercially available from GeneDetect (Auckland, New Zealand). AAV has been shown to have a high frequency of integration and infects nondividing cells, thus making it useful for delivery of genes into mammalian cells in tissue culture (Muzyczka, Curr Top Microbiol Immunol, 158:97 129, 1992). AAV has a broad host range for infectivity (Tratschin et al., Mol. Cell. Biol., 4:2072 2081, 1984; Laughlin et al., J. Virol., 60(2):515 524, 1986; Lebkowski et al., Mol. Cell. Biol., 8(10):3988 3996, 1988; McLaughlin et al., J. Virol., 62(6):1963 1973, 1988).

Methods of constructing and using AAV vectors are described, for example in U.S. Pat. Nos. 5,139,941 and 4,797,368. Use of AAV in gene delivery is further described in LaFace et al., Virology, 162(2):483 486, 1988; Zhou et al., Exp. Hematol, 21:928 933, 1993; Flotte et al., Am. J. Respir. Cell Mol. Biol., 7(3):349 356, 1992; and Walsh et al., J. Clin. Invest, 94:1440 1448, 1994.

Recombinant AAV vectors have been used for in vitro and in vivo transduction of marker genes (Kaplitt et al., Nat. Genet., 8(2):148 54, 1994; Lebkowski et al., Mol. Cell. Biol., 8(10):3988 3996, 1988; Samulski et al., EMBO J., 10:3941 3950,1991; Shelling and Smith, Gene Therapy, 1: 165 169, 1994; Yoder et al., Blood, 82 (Supp.): 1:347 A, 1994; Zhou et al., Exp. Hematol, 21:928 933, 1993; Tratschin et al., Mol. Cell. Biol., 5:3258 3260, 1985; McLaughlin et al., J. Virol., 62(6):1963 1973, 1988) and transduction of genes involved in human diseases (Flotte et al., Am. J. Respir. Cell Mol. Biol., 7(3):349 356, 1992; Ohi et al., Gene, 89(2):279 282, 1990; Walsh et al., J. Clin. Invest, 94:1440 1448, 1994; and Wei et al., Gene Therapy, 1:261268, 1994).

The expression of the engineered RNA precursors is driven by regulatory sequences, and the vectors of the invention can include any regulatory sequences known in the art to act in mammalian cells, e.g., murine cells; in insect cells; in plant cells; or other cells. The term regulatory sequence includes promoters, enhancers, and other expression control elements. It will be appreciated that the appropriate regulatory sequence depends on such factors as the future use of the cell or transgenic animal into which a sequence encoding an engineered RNA precursor is being introduced, and the level of expression of the desired RNA precursor. A person skilled in the art would be able to choose the appropriate regulatory sequence. For example, the transgenic animals described herein can be used to determine the role of a test polypeptide or the engineered RNA precursors in a particular cell type, e.g., a hematopoietic cell or bacterial cell. In this case, a regulatory sequence that drives expression of the transgene ubiquitously, or a specific regulatory sequence that expresses the transgene only in those cells, can be used. Expression of the engineered RNA precursors in a cell means that the cell is now susceptible to specific, targeted RNAi of a particular gene. Examples of various regulatory sequences are described herein.

The regulatory sequences can be inducible or constitutive. Suitable constitutive regulatory sequences include the regulatory sequence of a housekeeping gene such as the α-actin regulatory sequence, or may be of viral origin such as regulatory sequences derived from mouse mammary tumor virus (MMTV) or cytomegalovirus (CMV).

Alternatively, the regulatory sequence can direct transgene expression in specific organs or cell types (see, e.g., Lasko et al., 1992, Proc. Natl. Acad. Sci. USA 89:6232). Several tissue-specific regulatory sequences are known in the art including the albumin regulatory sequence for liver (Pinkert et al., 1987, Genes Dev. 1:268-276); the endothelin regulatory sequence for endothelial cells (Lee, 1990, J. Biol. Chem. 265:10446-50); the keratin regulatory sequence for epidermis; the myosin light chain-2 regulatory sequence for heart (Lee et al., 1992, J. Biol. Chem. 267:15875-85), and the insulin regulatory sequence for pancreas (Bucchini et al., 1986, Proc. Natl. Acad. Sci. USA 83:2511-2515), or the vav regulatory sequence for hematopoietic cells (Oligvy et al., 1999, Proc. Natl. Acad. Sci. USA 96:14943-14948). Another suitable regulatory sequence, which directs constitutive expression of transgenes in cells of hematopoietic origin, is the murine MHC class I regulatory sequence (Morello et al., 1986, EMBO J. 5:1877-1882). Since MHC expression is induced by cytokines, expression of a test gene operably linked to this regulatory sequence can be upregulated in the presence of cytokines.

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals such as mice, include regulation by ecdysone, by estrogen, progesterone, tetracycline, chemical inducers of dimerization, and isopropyl-beta-D-1 thiogalactopyranoside (IPTG) (collectively referred to as “the regulatory molecule”). Each of these expression systems is well described in the literature and permits expression of the transgene throughout the animal in a manner controlled by the presence or absence of the regulatory molecule. For a review of inducible expression systems, see, e.g., Mills, 2001, Genes Devel. 15:1461-1467, and references cited therein.

The regulatory elements referred to above include, but are not limited to, the cytomegalovirus hCMV immediate early gene, the early or late promoters of SV40 adenovirus (Bernoist et al., Nature, 290:304, 1981), the tet system, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast .alpha.-mating factors. Additional promoters include the promoter contained in the 3′ long terminal repeat of Rous sarcoma virus (Yamamoto et al., Cell 22:787-797, 1988); the herpes thymidine kinase promoter (Wagner et al., Proc. Natl. Acad. Sci. USA 78:1441, 1981); or the regulatory sequences of the metallothionein gene (Brinster et al., Nature 296:39, 1988).

The compositions, methods, kits and devices herein show compositions, methods and kits for the early diagnosis of virulent Gram-negative bacteria, and also compositions, methods and kits for treating or preventing a disease associated with Gram-negative bacteria using a modulator that binds to and inhibits function and/or expression of a virulence factor associated with the bacteria. The modulator in certain embodiments is a protein that binds to the virulence factor or that binds to a gene having a nucleotide sequence that encodes the virulence factor. Methods are shown for identifying the virulence factor using genetic screening, and for preparing a modulator that inhibits the function and expression of the virulence factor. In certain embodiments, the modulator is an RNA precursor. In certain embodiments the RNA precursors are expressed in a cell, for example using a viral vector or a bacterial vector. In certain embodiments the RNA precursors are processed by the cell to produce targeted small interfering RNAs (siRNAs) that selectively silence targeted genes and/or a portion thereof.

### Antibodies

The present invention relates also to compositions, methods and kits treating or preventing a disease associated with Gram-negative bacteria using in various embodiments a modulator. The modulator in many embodiments includes a binding agent that is an antibody that selectively binds the virulence factor. The term “antibody” as referred to herein includes whole antibodies and any antigen binding fragment (i.e., “antigen-binding portion”) or single chains of these. A naturally occurring “antibody” is a glycoprotein including at least two heavy (H) chains and two light (L) chains inter-connected by disulfide bonds.

As used herein, an antibody that “specifically binds to a virulence factor” is intended to refer to an antibody that binds to a domain or portion of the virulence factor having an amino acid sequence or nucleotide sequence. For example the antibody has a K_{D} of 5×10⁻⁹ M or less, 2×10⁻⁹ M or less, or 1×10⁻¹⁰ M or less. For example, the antibody is monoclonal or polyclonal. The terms “monoclonal antibody” or “monoclonal antibody composition” as used herein refer to a preparation of antibody molecules of single molecular composition. A monoclonal antibody composition displays a single binding specificity and affinity for a portion of a Gram negative bacterial cell for example a particular epitope of the cell such as a MrtAB protein. The antibody is an IgM, IgE, IgG such as IgG1 or IgG4.

Also useful for systems, method and kits herein is an antibody that is a recombinant antibody. The term “recombinant human antibody”, as used herein, includes all antibodies that are prepared, expressed, created or isolated by recombinant means, such as antibodies isolated from an animal (e.g., a mouse). Mammalian host cells for expressing the recombinant antibodies used in the methods herein include Chinese Hamster Ovary (CHO cells) including dhfr-CHO cells, described Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. To produce antibodies, expression vectors encoding antibody genes are introduced into mammalian host cells, and the host cells arc cultured for a period of time sufficient to allow for expression of the antibody in the host cells or secretion of the antibody into the culture medium in which the host cells are grown. Antibodies can be recovered from the culture medium using standard protein purification methods.

Standard assays to evaluate the binding ability of the antibodies toward the target of various species are known in the art, including for example, ELISAs, western blots and RIAs. The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as goats, dogs, sheep, mice, or camels are immunized by administration of an amount of immunogen, such as the intact protein or a portion thereof containing an epitope from a Gram-negative bacterial strain, effective to produce an immune response. In certain embodiments, the animal is subcutaneously injected in the back with 100 micrograms to 100 milligrams of antigen, dependent on the size of the animal, followed three weeks later with an intraperitoneal injection of 100 micrograms to 100 milligrams of immunogen with adjuvant dependent on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing the virulence factor or a portion thereof.

The technique of in vitro immunization of human lymphocytes is used to generate monoclonal antibodies. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies. Crystal et al., U.S. Pat. No. 7,863,425 issued Jan. 4, 2011, and Hill et al., U.S. Pat. No. 7,572,449 issued Aug. 11, 2009 are each incorporated by reference herein in their entireties.

### RNA Interference

The present invention further encompasses compositions, methods and kits useful for suppressing or selectively eliminating virulence factors and/or for immunizing a subject to the virulence factors. The present invention provides agents for treating a subject having been exposed to a Gram-negative bacterial strain associated with the virulence factor or preventing the pathological symptoms of virulence factors including contacting a subject with an agent that negatively modulates the virulence factor. For example contacting the subject includes contacting with a virus vector that expresses the agent including a negative modulator of expression of the virulence factor, or contacting involves administering a recombinant bacterial strain lacking the virulence factor.

The present invention provides a method of treating a human subject having been exposed to a bacterial strain having a virulence factor or at risk for being exposed to the bacterial strain, the method including: constructing a negative modulator of a virulence factor protein or of a nucleic acid encoding a region having the virulence factor; and contacting at least one of cells or tissue of the subject with the modulator that down regulates or eliminates expression of the region and/or the virulence factor, thereby treating the subject.

The methods herein include engineering the modulator by constructing a small interfering RNA (siRNA) that specifically targets a nucleic acid encoding the region having the virulence factor or encoding an agent that binds to the virulence factor.

The modulator in various embodiments of the method includes a nucleic acid vector or a virus vector that includes a nucleic acid sequence encoding a siRNA negative modulator of a target nucleic acid encoding the region, such that the siRNA down-regulates or interferes with the function of mRNAs encoding the virulence factor. In certain embodiments, the virus vector includes a nucleic acid sequence encoding an antisense RNA modulator of a target nucleic acid, for example the target nucleic acid includes a conserved domain or a polymorphic domain of a gene carried by a Gram-negative strain. Alternatively, the vector is a bacterial vector lacking the region or domain of a gene encoding the virulence factor, e.g., a MrtAB protein or a gene encoding the MrtAB protein.

Methods for constructing synthetic siRNA or an antisense expression cassette and inserting it into a recombinantly engineered nucleic acid of a vector are well known in the art and are shown for example in Reich et al. U.S. Pat. No. 7,847,090 issued Dec. 7, 2010; Reich et al. U.S. Pat. No. 7,674,895 issued Mar. 9, 2010; and Khvorova et al. U.S. Pat. No. 7,642,349 issued Jan. 5, 2010, each of which is incorporated herein in its entirety. For example, the invention herein includes synthetic siRNAs that include a sense RNA strand and an antisense RNA strand, such that the sense RNA strand includes a nucleotide sequence substantially identical to a target nucleic acid sequence in cells. Thus, under the circumstances of cells being contacted with viral vectors encoding the siRNAs, the cells express the siRNAs that then negatively modulate expression of the target nucleic acid sequence, e.g., a target nucleotide sequence in a mrtAB gene.

Methods and compositions for targeting and negatively modulating regions in pathogens and organisms are shown also in Zamora et al., U.S. Pat. No. 7,772,203 issued Aug. 10, 2010; Zamora et al. U.S. Pat. No. 7,893,036 issued Feb. 22, 2011; and Zamora et al., U.S. patent number 8,304,530 issued Nov. 6, 2012, each of which is incorporated herein in its entirety. In certain embodiments, the modulator, for example the siRNA, is permanently integrated into a genome of the cells or tissue of the subject. Methods for identifying presence of conserved or polymorphic domains and regions, methods for designing and constructed RNA interference agents that negatively modulate expression of the domain or domains of proteins, and methods for preparing transplants, donor cells, or graft materials are shown for example in McSwiggen et al., U.S. Pat. No. 7,176,304 issued Feb. 13, 2007; Cicciarelli et al., U.S. Pat. No. 8,236,771 issued Aug. 7, 2012; and Trono et al., U.S. patent application number 2005/0014166 published Jan. 20, 2005. Methods of producing libraries or banks of cells (e.g., stem cells, helper cells, and donor cells) are shown for example in West, U.S. patent publication number 2004/0091936 published May 13, 2004; and Crawford et al., international patent publication number WO/2011/041240 published Apr. 7, 2011. All of references, issued patents, patent publications and international applications identified herein are hereby incorporated by reference herein in their entireties.

### Pharmaceutical Compositions

An aspect of the present invention provides pharmaceutical compositions that include either an attenuated form of a bacterial pathogen, or a modulator that selectively binds or negatively modulates expression of a virulence factor that is associated with a Gram-negative bacterial disease or condition. In related embodiments, the pharmaceutical composition is formulated sufficiently pure for administration to a subject, e.g., a human or a non-human such as a mouse, a rat, a dog, a cat, and a cow. The pharmaceutical composition is administered for example to an abdomen such as a liver, spleen, or kidney; an appendage; a lymph node; or vascular system.

In certain embodiments, the pharmaceutical composition further includes at least one therapeutic agent selected from the group consisting of: anti-bacterial agent, anti-fungal agent, growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid. See Toole et al. U.S. Pat. No. 5,902,795 issued May 11, 1999, which is incorporated by reference herein in its entirety.

The therapeutic agent in various embodiments includes an anti-cancer or anti-tumor agent selected from the group of: alkylating agents, such as mechlorethamine, cyclophosphamide, melphalan, uracil mustard, chlorambucil, busulfan, carmustine, lomustine, semustine, streptozoticin, and decrabazine; antimetabolites, such as methotrexate, fluorouracil, fluorodeoxyuridine, cytarabine, azarabine, idoxuridine, mercaptopurine, azathioprine, thioguanine, and adenine arabinoside; natural product derivatives, such as irinotecan hydrochloride, vinblastine, vincristine, dactinomycin, daunorubicin, doxorubicin, mithramycin, taxanes (e.g., paclitaxel) bleomycin, etoposide, teniposide, and mitomycin C; and miscellaneous agents, such as hydroxyurea, procarbazine, mititane, and cisplatinum. See Brown et al. U.S. publication number 20050267069 published Dec. 1, 2005, which is incorporated by reference herein in its entirety.

In other embodiments, the therapeutic agent is a cell, a compound, a composition, biological or the like that potentiates, stabilizes or synergizes the effects of the modulator or another molecule or compound on a cell or tissue. In some embodiments, the drug may include without limitation anti-tumor, anti-viral, antibacterial, anti-mycobacterial, anti-fungal, anti-proliferative or anti-apoptotic agents. Drugs that are included in the compositions of the invention are well known in the art. See for example, Goodman & Gilman's The Pharmacological Basis of Therapeutics, 9th Ed., Hardman, et al., eds., McGraw-Hill, 1996, the contents of which are herein incorporated by reference herein.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, the choice of agents and non-irritating concentrations to be determined according to the judgment of the formulator.

### Therapeutically Effective Dose

Methods provided herein involves contacting a subject with a pharmaceutical composition, for example, administering a therapeutically effective amount of a pharmaceutical composition having a vaccine which is an attenuated mutant of a bacterial pathogen, or is a modulator that selectively binds a virulence factor, or a gene that encodes an inactive form of the virulence factor, and optionally further having a therapeutic agent, to a subject in need thereof, in such amounts and for such time as is necessary to achieve the desired result.

The compositions, according to the method of the present invention, may be administered using any amount and any route of administration effective for treating a subject. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., intermediate or advanced stage of the disease associated with the Gram-negative pathogen; age, weight and gender of the patient; diet, time and frequency of administration; route of administration; drug combinations; reaction sensitivities; and tolerance/response to therapy. Long acting pharmaceutical compositions might be administered hourly, twice hourly, every three to four hours, daily, twice daily, every three to four days, every week, or once every two weeks depending on half-life and clearance rate of the particular composition and/or modulator.

The active agents of the invention, such as the vaccine or the modulator, are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, as provided herein, usually mice, but also potentially from rats, rabbits, dogs, or pigs. The infected animal model described herein is also used to achieve a desirable concentration and total dosing range and route of administration. Such information can then be used to determine useful doses and routes for administration in humans or non-humans, e.g., high value animals, farm animals, or pets.

A therapeutically effective dose refers to that amount of active agent that ameliorates the symptoms or prevents progression of pathology or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies are used in formulating a range of dosage for human use.

### Administration of Pharmaceutical Compositions

As formulated with an appropriate pharmaceutically acceptable carrier in a desired dosage, the pharmaceutical composition including the attenuated bacterial strain vaccine, or the modulator provided herein is administered to humans and other mammals topically such as ocularly (as by solutions, ointments, or drops), nasally, bucally, orally, rectally, topically, transdermally, parenterally, intracisternally, intravaginally, or intraperitoneally.

Injections include intravenous injection, direct or parental injection into a tissue or organ, or injection into the external layers of the tissue or organ or adjacent tissues, such as injection into the peritoneal cavity.

The pharmaceutical composition in various embodiments is administered with inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the delivered compositions can also include adjuvants such as wetting agents, and emulsifying and suspending agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous routes of administration are achieved with aqueous drops, a mist, an emulsion, or a cream. Administration may be diagnostic, prognostic, therapeutic or it may be prophylactic. The invention includes delivery devices, surgical devices, audiological devices or products which contain disclosed compositions (e.g., gauze bandages or strips), and methods of making or using such devices or products. These devices may be coated with, impregnated with, bonded to or otherwise treated with a composition as described herein.

Transdermal patches have the added advantage of providing controlled delivery of the active ingredients to the body. Such dosage forms can be made by dissolving or dispensing the compound or composition including the modulator in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions used to prepare a live attenuated bacterial vaccine, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For the purpose any bland fixed oil can be employed including synthetic mono- or diglycerides. In addition, fatty acids such as oleic acid are used in the preparation of injectables. The injectable formulations prior to addition of the attenuated bacteria can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of the invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

Numerous embodiments of the invention are provided herein are found in publication in PLoS Pathogens (Public Library of Science, Pathogens) entitled “Identification of MrtAB, an ABC Transporter Specifically Required for Yersinia pseudotuberculosis to Colonize the Mesenteric Lymph Nodes” by Gregory T. Crimmins, Sina Mohammadi, Erin R. Green, Molly A. Bergman, Ralph R. Isberg, and Joan Mccsas (Crimmins, G. T. et al. 2012PLoS Pathog 8(8): e1002828). This publication and supplementary material, and U.S. provisional application Ser. No. 61/656,640 filed Jun. 7, 2012 entitled, “Methods, compositions and kits for treating or preventing a disease associated with Gram-negative bacteria” by Joan Mecsas, Gregory T. Crimmins, Sina Mohammadi, Erin R. Green and Ralph R. Isberg, are each hereby incorporated herein by reference in their entireties.

A skilled person will recognize that many suitable variations of the methods may be substituted for or used in addition to those described above and in the claims. It should be understood that the implementation of other variations and modifications of the embodiments of the invention and its various aspects will be apparent to one skilled in the art, and that the invention is not limited by the specific embodiments described herein and in the claims. Therefore, it is contemplated to cover the present embodiments of the invention and any and all modifications, variations, or equivalents that fall within the true spirit and scope of the basic underlying principles disclosed and claimed herein. All of references, issued patents, patent publications and international applications identified herein are hereby incorporated by reference herein in their entireties.

### EXAMPLES

### Example 1

### Bacterial Strains and Genetics

Yersinia pseudotuberculosis (Yptb) strains used in this study were derived from strain YPIII (Balada-Llasat, J. M. et al. 2006 PLoS Pathog 2: e86). In frame deletions were generated using pCVD442 and 500-800 base pairs (bps) upstream and downstream of the DNA to be removed (Ibid.). Primer sequences used to generate the mrtAB knockout construct were the following: mrtAB FORT: attaGCATGCTTGCTGGAAACGTTTAAAGCGTTTGG (SEQ ID NO: 18), mrtAB REV1:attaGAATTCTAATTGTGCAAACAATCTCACGCAGTTT (SEQ ID NO: 19), mrtAB FOR2: attaGAATTCAGGAGGTCGAAGC CGATGAATAAC (SEQ ID NO: 20), mrtAB REV2: attaGAGCTCTTGAAA TCAGCGCCATCCGCCAAT (SEQ ID NO: 21). For HA tagging of mrtA (YPK_{—}3222; SEQ ID NO: 2), the HA sequence was inserted directly downstream of the ATG start codon of the operon. For the FLAG tagging of mrtB (YPK_{—}3221; SEQ ID NO: 1), the FLAG sequence was inserted immediately upstream of the stop codon. The coding regions of the two genes are overlapping, and tags in the C terminus of MrtA or the N terminus of MrtB were constructed. Yptb regions were tagged with GFP by driving expression of GFP by the constitutive Tet promoter on pACYC 184. The tetA::GFP promoter-gene fusion from pDWS (Cummings, L. A. et al. 2006 Mol Microbiol 61: 795-809) was PCR-amplified with SphI end sites and moved it into pACYC184 cut with SphI. Forward primer: 5′ gatcgcatgcgaattctcatgtttgacagcttat 3′(SEQ ID NO: 22); Reverse primer: 5′ gccgccgcaaggaatggtgcatgc (SEQ ID NO: 23). The resulting plasmid was very stable in vivo.

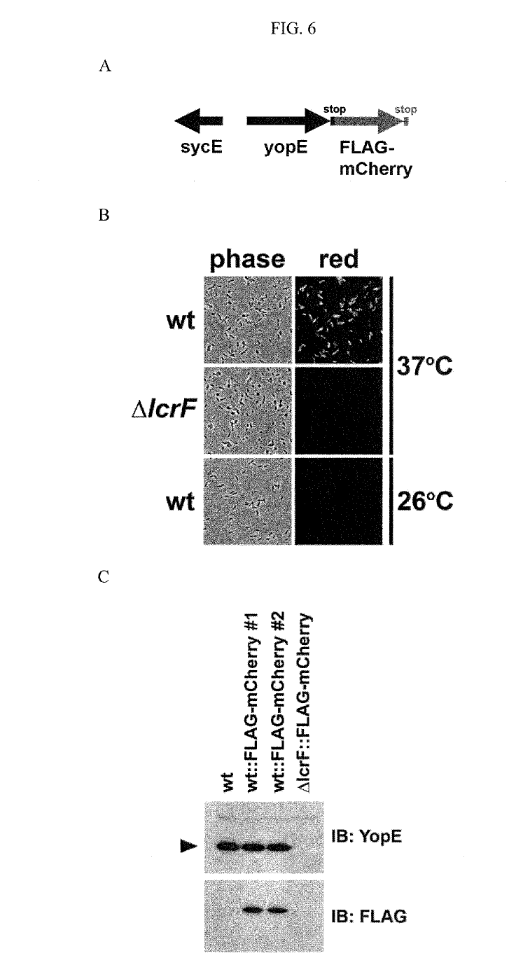

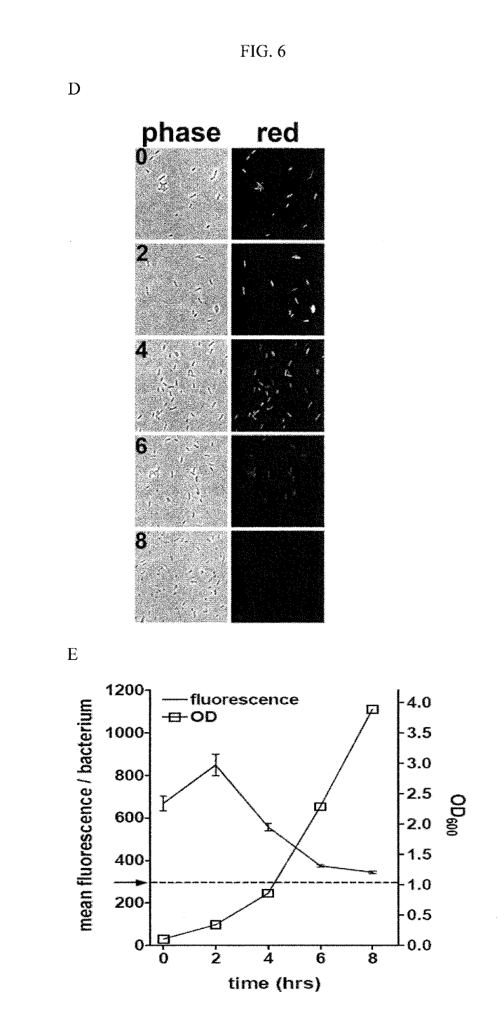

For the construction of the mrtAB complementation plasmid (pmrtAB), pACYC 184 was digested with EcoRV and SalI, and the mrtAB operon was PCR-amplified with EcoRV and SalI end sites. The entire intergenic sequence between YPK_{—}3223 and YPK_{—}3222 (101 bps) was included upstream of the mrtA start codon, and the mrtB terminator was included after the gene. The primers used for the complementation vector were: CompFor: attaTCTAGAATAATTCACTAAAAAATCTGTTTATCAATGGT (SEQ ID NO: 24), and CompRev: attaGTCGACAAGTGA GTGAGTGAGTGAGTGAGT (SEQ ID NO: 25). A YopE reporter strain was constructed with a FLAG-mCherry sequence immediately following the yopE stop codon. An isogenic, unmarked T3SS reporter strain was constructed that contains FLAG-mCherry sequence immediately after the yopE stop codon (see FIG. 6 panel A). A DNA fragment containing the FLAG-mCherry sequence, flanked by about a one kilobase (kb) of genomic sequence on each side of yopE stop codon was constructed by PCR and cloned into the Sad and BamHI sites of pSR47s. The resulting plasmid (pSR47s-yopE-FLAG-mCherry) was introduced into E. coli DH5α λpir and integrated onto the Y. pseudotuberculosis virulence plasmid by triparental mating using the helper E. coli strain IIB101(RK600).

### Example 2

### Media and Growth Conditions

Yptb cultures were grown in 2XYT broth (Sigma-Aldrich; St. Louis, Mo.), except for cultures used in determination of the MIC as described herein. Kanamycin (30 micrograms per milliliter; μg/ml) for selection of transposon, and Irgasan (2 μg/ml) for selection for Yptb were used in production of Yptb transposon mutant libraries. Chloramphenicol (25 μg/ml) was used in selection for pACYC184 derived complementation plasmids. For intravenous (IV), oral, or intraperitoneal (IP) infections, Yptb cultures were grown at 26° C. overnight with rolling, prior to injection/administration to subjects. For in vitro growth for measuring MrtB protein levels by Western blot, bacteria were grown overnight in LB medium, and were diluted (1:40) the following morning in LB. Cultures were grown 90 minutes at 26° C., then half the samples were transferred to 37° C. and half were left at 26° C., and were grown for an additional 90 minutes prior to protein isolation. Mouse anti-FLAG antibody was used as a primary antibody (overnight at 4° C.) and Cy5-labeled goat anti-mouse antibody was used as a secondary antibody. Western blots were visualized using a Fuji FLA-9000 image scanner (FujiFilm Corporation; Tokyo, Japan).

### Example 3

### Generation of Mariner Transposon Mutant Libraries in Yptb (P⁻)

Vector pSC189 containing Himar1 (Chiang, S. L. et al. 2002 Gene 296: 179-185) was mutated on one end of the transposon recognition sequence to produce an Mmel restriction site (van Opijnen, T. et al. 2009 Nat Methods 6: 767-772). To perform transpositions in a Yptb strain, the Himar1 (MmeI) transposon was introduced into YPIII(P⁻) by mating with SM10λpir. Approximately 25 ml of YPIII(P⁻) was grown overnight in 2XYT broth at 26° C., and 75 ml of SM10λpir(pSC189Himar1MmeI)) was grown overnight at 37° C. in LB medium containing 30 μg/ml kanamycin and 100 μg/ml ampicillin. The SM10λpir cultures were washed three times with PBS, pelleted, and re-suspended in the YPIII(P⁻) culture medium. Mating was conducted for 16 hours to 24 hours at 37° C. in the spent Yptb culture medium. Cells were then pelleted, re-suspended in 2XYT broth (5 ml), and spread on ten LB plates containing 30 μg/ml kanamycin and 2 μg/ml irgasin. Libraries of cells obtained from approximately 10,000 colonies were prepared by scraping cells from the plates, pelleting, re-suspending in 50% glycerol and storing at −80° C.

### Example 4

### Genetic Screens

Libraries of 10,000 Himar1 mutants were adjusted to yield a total of 200,000 colonies on LB medium, and cells were scraped and were re-suspended in 2XYT broth. Small aliquots were used to start overnight cultures (26° C.) in 2XYT broth. Mice were injected in the tail vein with 1×10⁵ bacteria. Organs were excised and homogenized at various times post infection. Bacteria were isolated by plating homogenized samples on LB medium containing 30 μg/ml kanamycin and 1 μg/ml irgasan. Colonies were removed from plates and genomic DNA was isolated using Qiagen DNeasy kit. Samples were prepared for Illumina sequencing (van Opijnen, T. et al. 2009 Nat Methods 6: 767-772; Opijnen, T. et al. 2010 Curr Protoc Microbiol Chapter 1: UnitlE 3). For 26° C. compared to 37° C. screenings, both 10,000 Himar1 mutant libraries were plated and combined. Overnight cultures were grown at 26° C., diluted into 2XYT broth the following day, and grown overnight at either 26° C. or 37° C. The temperature selection screen of the cells was thus an analysis for both growth and stationary phase at 37° C.

Screen data analysis was performed by plating out each overnight culture of an individual library of 10,000 transposon mutants to obtain more than 200,000 colonies. The colonies were then used to generate genomic DNA which is defined herein as an Input sample. For each library, there were at least two Input samples, with a corresponding set of Output samples, defined as the colonies from an individual infected liver derived from a given Input injection dose. After Illumina sequencing, the number of reads/hits for each gene in the Output sample was normalized for amount of DNA added to sequencing run (total number of reads) and normalized for the number of unique insertions in a particular pool. This calculation produced a value for each gene, specifically the value which corresponds to the relative abundance of clones containing a transposon insertion in a given gene X within the pool. Insertions located within the first 5% or last 10% of a gene were discarded and the remaining values for insertions within a single gene were summed.

Table 1 contains data identifying mutants defective for colonization in liver samples. Ten thousand mutants were screened through ten murine subjects, for a total of more than 20,000 independent transposon insertion mutants, encompassing 3,088 genes (FIG. 2 panel A). Table 1 lists genes encoding different functional categories shown in the left column of the table: known virulence factors; amino acid and purine synthesis; lipopolysaccharides (LPS) modification; and novel candidate virulence factors. Table 1 lists for each gene a nucleotide sequence and an amino acid sequence, respectively. For example the nucleotide sequence of YPK_{—}3221 is SEQ ID NO: 1 and the amino acid sequence is SEQ ID NO: 58, and the nucleotide sequence of YPK_{—}32212 is SEQ ID NO: 2 and the amino acid sequence is SEQ ID NO: 59.

TABLE 1
Identification of mutants defective for colonization in the liver

| | | | | | | | | SEQ ID |
|---|---|---|---|---|---|---|---|---|
| Functions | Gene | Library | Annotation | Output/Input | | 37/26 | | NOs.: |
| known | YPK_2757 | #1 and #2 | pH 6 Ag | 1.07E−02 | (−4.72) | 1.15 | (0.39) | 27; 75 |
| virulence | YPK_0665 | #1 and #2 | sufI | 1.86E−02 | (−3.99) | 0.38 | (−1.95) | 28; 76 |
| factors | YPK_2429 | #1 and #2 | invasin | 1.98E−02 | (−3.91) | 1.23 | (0.53) | 29; 77 |
| | YPK_2759 | #1 and #2 | pH 6 Ag | 3.08E−02 | (−3.32) | 0.83 | (−0.29) | 30; 78 |
| | YPK_2758 | #2 | ph 6 Ag | 0 | (NA) | ND | | 31; 79 |
| amino acid | YPK_0321 | #1 and #2 | aroE | 3.28E−04 | (−9.27) | 0.23 | (−3.05) | 32; 80 |
| and purine | YPK_1253 | #1 and #2 | purM | 5.28E−03 | (−5.67) | 0.96 | (0.002) | 33; 81 |
| synthesis | YPK_0226 | #1 and #2 | aroB | 1.29E−02 | (−4.48) | 0.28 | (−2.62) | 34; 82 |
| | YPK_0357 | #1 and #2 | purH | 1.80E−02 | (−4.04) | 0.66 | (−0.8) | 35; 83 |
| | YPK_2670 | #1 and #2 | aroA | 3.06E−02 | (−3.33) | 0.44 | (−1.65) | 36; 84 |
| | YPK_2047 | #1 and #2 | trpA | 3.33E−02 | (−3.22) | 0.61 | (−1.0) | 37; 85 |
| | YPK_2528 | #1 and #2 | hisB | 3.42E−02 | (−3.18) | 0.87 | (−0.22) | 38; 86 |
| | YPK_0356 | #1 | purD | 9.53E−03 | (−2.58) | 0.82 | (−0.33) | 39; 87 |
| | YPK_1364 | #2 | purC | 0 | (NA) | 0.57 | (1.1) | 40; 88 |
| LPS | YPK_3181 | #1 and #2 | O—Ag | 0 | (NA) | 0.22 | (−3.17) | 41; 89 |
| modification | YPK_3646 | #1 and #2 | waaL | 0 | (NA) | 0.02 | (−8.55) | 42; 90 |
| | YPK_4033 | #1 and #2 | wecA | 0 | (NA) | 0.0008 | (−15.03) | 43; 91 |
| | YPK_3937 | #1 and #2 | rfaH | 2.36E−05 | (−12.87) | 0.52 | (−1.31) | 44; 92 |
| | YPK_3184 | #1 and #2 | O—Ag | 1.52E−03 | (−7.32) | 0.61 | (−0.95) | 45; 93 |
| | YPK_3190 | #1 and #2 | O—Ag | 1.98E−03 | (−6.97) | 0.24 | (−2.91) | 46; 94 |
| | YPK_3179 | #1 and #2 | O—Ag | 2.32E−03 | (−6.77) | 1.09 | (0.27) | 47; 95 |
| | YPK_3183 | #1 and #2 | O—Ag | 5.38E−03 | (−5.64) | 0.37 | (−2.02) | 48; 96 |
| | YPK_3182 | #1 and #2 | O—Ag | 8.05E−03 | (−5.11) | 0.23 | (−3.06) | 49; 97 |
| | YPK_3189 | #1 and #2 | O—Ag | 2.05E−02 | (−3.87) | 0.15 | (−4.0) | 50; 98 |
| | YPK_4030 | #1 and #2 | wecC | 2.61E−02 | (−3.55) | 1.03 | (0.16) | 51; 99 |
| | YPK_1834 | #1 and #2 | arnD | 4.77E−02 | (−2.74) | 0.69 | (−0.71) | 52; 100 |
| | YPK_3180 | #1 | O—Ag | 8.83E−03 | (−2.64) | 0.16 | (−3.74) | 53; 101 |
| | YPK_3188 | #2 | O—Ag | 2.63E−04 | (−5.42) | 0.25 | (−2.86) | 54; 102 |
| candidate | YPK_3221 | #1 and #2 | mrtB | 0 | (NA) | 0.73 | (−0.58) | 1; 58 |
| virulence | YPK_3222 | #1 and #2 | mrtA | 2.19E−03 | (−6.84) | 0.77 | (−0.46) | 2; 59 |
| factors | YPK_1234 | #1 and #2 | phage protein | 2.69E−03 | (−6.57) | 0.68 | (−0.73) | 3; 60 |
| | YPK_2423 | #1 and #2 | flgD | 3.03E−02 | (−3.34) | 1.03 | (0.16) | 4; 61 |
| | YPK_1292 | #1 and #2 | rodZ | 4.07E−02 | (−2.95) | 0.48 | (−1.45) | 5; 62 |
| | YPK_2066 | #1 and #2 | oppD | 4.40E−02 | (−2.85) | 1.05 | (0.19) | 6; 63 |
| | YPK_3575 | #1 and #2 | apaH | 4.48E−02 | (−2.82) | 0.18 | (−3.6) | 7; 64 |
| | YPK_1713 | #1 and #2 | Hypothetical | 5.20E−02 | (−2.63) | 1.05 | (0.2) | 8; 65 |
| | YPK_2406 | #1 | Hypothetical | 0 | (NA) | 1.75 | (1.28) | 9; 66 |
| | YPK_3656 | #1 | Hypothetical | 0 | (NA) | 0.78 | (−0.45) | 10; 67 |
| | YPK_0453 | #1 | tRNA synthase | 1.73E−04 | (−5.98) | 1.59 | (1.08) | 11; 68 |
| | YPK_0688 | #1 | Hypothetical | 4.46E−04 | (−5.17) | ND | | 12; 69 |
| | YPK_2424 | #1 | FlgC | 8.56E−03 | (−2.67) | 2.26 | (1.83) | 13; 70 |
| | YPK_3600 | #1 | Hypothetical | 9.96E−03 | (−2.54) | ND | | 14; 71 |
| | YPK_2199 | #2 | Hypothetical | 0 | (NA) | 0.85 | (−0.27) | 15; 72 |
| | YPK_4078 | #2 | sthA | 6.57E−03 | (−2.96) | 0.3 | (−2.49) | 16; 73 |
| | YPK_0208 | #2 | Hypothetical | 8.67E−03 | (−2.75) | 0.85 | (−0.26) | 17; 74 |

Data in Table 1 identify gene insertions that were greater or equal to 2.5 standard deviations (s.d.) depleted from the pool relative to the mean based on number of sequencing reads described in the experimental procedures herein. The column identified as “Library” lists the library harboring the mutations in the corresponding gene. The column identified as “Output/Input” shows the average ratio of the relative abundance of clones containing transposon insertions in genes in the Output liver sample/Input liver sample (±sd). The column labeled “ 37/26” shows the average ratio of the relative abundance of clones containing transposon insertions in the gene after growth in broth at 37° C./growth in broth at 26° C. (±sd).

Table 1 contains data identifying mutants defective for colonization in liver samples. Ten thousand mutants were screened through ten murine subjects, for a total of more than 20,000 independent transposon insertion mutants, encompassing 3,088 genes (FIG. 2 panel A). Table 1 lists genes encoding different functional categories shown in the left column of the table: known virulence factors; amino acid and purine synthesis; lipopolysaccharides (LPS) modification; and novel candidate virulence factors. Table 1 lists for each gene a nucleotide sequence and an amino acid sequence, respectively. For example the nucleotide sequence of YPK_{—}3221 is SEQ ID NO: 1 and the amino acid sequence is SEQ ID NO: 58, and the nucleotide sequence of YPK_{—}32212 is SEQ ID NO: 2 and the amino acid sequence is SEQ ID NO: 59.

Data in Table 1 identify gene insertions that were greater or equal to 2.5 standard deviations (s.d.) depleted from the pool relative to the mean based on number of sequencing reads described in the experimental procedures herein. The column identified as “Library” lists the library harboring the mutations in the corresponding gene. The column identified as “Output/Input” shows the average ratio of the relative abundance of clones containing transposon insertions in genes in the Output liver sample/Input liver sample (±sd). The column labeled “37/26” shows the average ratio of the relative abundance of clones containing transposon insertions in the gene after growth in broth at 37° C./growth in broth at 26° C. (±sd).

TABLE 2
ATPase activity of MrtB is required for resistance to ethidium bromide

| | cell genotype: | | | | | |
|---|---|---|---|---|---|---|
| | | Yptb(P⁻) | Yptb(P⁻) | | Yptb(P⁻) | Yptb(P⁻) |
| | Yptb(P⁻) | ΔmrtAB | ΔmrtAB | Yptb(P⁻) | ΔmrtAB | ΔmrtAB |
| Agent | pGC1 (empty) | pGC1 (empty) | pmrtA⁺B⁺ | pmrtA⁺B⁺ | pmrtA⁺B⁺ flag | pmrtA⁺B*- flag |
| ethidium bromide | 25 | 25 | 100 | 100 | 100 | 50 |
| (EtBr) | | | | | | |
| aeridine orange | 50 | 50 | 50 | 50 | ND | ND |
| (Acr orange) | | | | | | |
| pyocyanin | 1 | 1 | 0.25 | 0.25 | 0.25 | 2 |

Data are displayed as minimum inhibitory concentration (MIC in μg/mL), defined as: lowest concentration of toxic compound that resulted in less than half maximal growth in an overnight culture incubated without shaking, at 37° C.
⁺indicates wild type gene, and
*indicates a gene with a point mutation in the Walker A box of the ATPase domain.

The values for each gene in the Output liver samples in Table 1 were then divided by the values in the corresponding Input sample. This calculation yields a ratio of the relative abundance of clones containing a transposon insertion in gene X in the Output liver sample, divided by the relative abundance of clones containing a transposon insertion in gene X in the Input sample. The Log₂ value of this ratio was used for further statistical analysis, including determining the average ratio and standard deviation (s.d.). Table 2 contains data showing that ATPase activity of MrtB is required for resistance to ethidium bromide.

Tables 1-6 herein are found also in PLOS Pathogen publication entitled “Identification of MrtAB, an ABC Transporter Specifically Required for Yersinia pseudotuberculosis to Colonize the Mesenteric Lymph Nodes” by Gregory T. Crimmins, Sina Mohammadi, Erin R. Green, Molly A. Bergman, Ralph R. Isberg, and Joan Mecsas (Crimmins, G. T. et al. 2012PLoS Pathog 8(8): e1002828), and U.S. provisional application Ser. No. 61/656,640 filed Jun. 7, 2012, each of which including supplementary data is hereby incorporated herein by reference in its entirety.

Table 3 contains screening data for positive hits affecting the same gene observed to be present in both libraries. Columns C-F list the number of reads for each gene in the Input samples normalized for the amount of DNA added to sequencing run (total number of reads) and normalized for the number of unique insertions in a particular pool. Columns G-AC list all the Output Liver samples, normalized as in columns C-F, then divided by the values in the corresponding Input sample. Columns AD-AM show the statistical analysis and annotation, including the average ratio of Output/Input, the Log₂ value of this ratio, the number of Standard Deviations away from the mean, and a reference to the 26° C. growth compared to 37° C. growth (Table 6).

Table 4 contains screening data from genes observed to have been mutated only in Library A. The analysis is similar to that in Table 3, including only data from genes hit Library A and not hit in Library B. Columns B and H list the number of reads for each gene in Library A Input sample normalized for the amount of DNA added to sequencing run (total number of reads) and normalized for the number of unique insertions in a particular pool. Columns C-G and I-M show the Output Liver samples, normalized as in columns B and H, then divided by the values in the corresponding Input sample. Columns O-U show the statistical analysis, including the average ratio of Output/Input, the Log₂ value of this ratio, and the number of Standard Deviations away from the mean.

Table 5 contains screening data from genes hit only in Library B. The analysis is similar to that in Table 3, including only data from genes hit Library B and not hit in Library A. Columns B and II show the number of reads for each gene in Library B Input sample normalized for the amount of DNA added to sequencing run (total number of reads) and normalized for the number of unique insertions in a particular pool. Columns C-G and I-P show the Output Liver samples, normalized as in B and H, then divided by the values in the corresponding Input sample. Columns Q-Y show the statistical analysis, including the average ratio of Output/Input, the Log₂ value of this ratio, and the number of Standard Deviations away from the mean.

Table 6 contains data for 26° C. growth compared to 37° C. growth in vitro. Libraries A and B were combined and grown overnight at 26° C., diluted into 2XYT medium broth the following day, and grown overnight at either 26° C. or 37° C. Columns B and C show the number of reads for each gene from samples grown at 26° C., normalized for the amount of DNA added to sequencing run (total number of reads) and normalized for the number of unique insertions in a particular pool. Columns D and E show a similar analysis to column B and C from samples grown at 37° C. Columns F-N show the statistical analysis, including the ratio of 26° C. values compared to 37° C. values, the Log₂ value of this ratio, and the number of standard deviations away from the mean.

### Example 5

### Mouse Infections and Histology

The subjects used in Examples herein were eight to ten weeks old mice. Animal subjects infected intravenously (IV) for subsequent analysis of CFU in organs were C57BL/6 strain of mice and CFU were analyzed at times and in organs as indicated. For Yptb (P⁻) bacteria testing, mice were infected intravenously with 1×10⁵ bacteria. For a control, mice were infected intravenously with 1×10³ wildtype bacteria. Oral infections were performed using BALB/c mice for ease in isolating the Peyer's patch (PP). Intraperitoneal infections were performed using BALB/c mice. For oral infections, food was removed from cages 16 hours before oral inoculation with 2×10⁹ Yptb bacteria. For Peyer's patch quantification of CFU, visible Peyer's patches from a single animal were combined prior to homogenization and plating. For small intestine CFU quantification, a five centimeter (cm) section of small intestine upstream of the cecum was removed. For both Peyer's patch and small intestine analyses, homogenates were plated using LB agar containing Irgasan® (triclosan; 1 μg/ml). For histology, inoculations with green fluorescent protein (GFP)-tagged bacteria were performed as all other inoculations, and organs were fixed in 4% paraformaldehyde for three hours, then flash frozen in Sub Xero freezing media (Mercedes Medical; Sarasota, Fla.). Ten micrometer (micron; μm) sections were excised from organs using a cryostat microtome, and sections were stained with Hoechst dyes (1:10,000 dilution). For neutrophil staining, monoclonal anti-Ly6G clone 1A8 antibody (BD Pharmingen; Franklin Lakes, N.J.) was used at a 1:100 dilution. For quantification of the YopE reporter strain, tissue sections were prepared as described herein, and were imaged using a Nikon AAR confocal imaging system (Nikon Instruments Inc.; Mellville, N.Y.). Images were quantified using an ImageJ processing program (Collins, T. J. 2007 BioTechniques 43 (1 Suppl): 25-30) with each microcolony analyzed for median mCherry fluorescence (normalized to median GFP), which was constitutively expressed.

Animal studies in Examples herein were performed in strict accordance with the recommendations in the Guide for the Care and Use of Laboratory Animals of the National Institutes of Health. The animal testing protocol was approved by the Tufts University Institutional Animal Care and Use Committee (IACUC). All efforts were made to minimize suffering including carefully monitoring animals following infection and euthanizing prior to or directly upon exhibiting substantial signs of morbidity. Animals were euthanized by carbon dioxide asphyxiation followed by cervical dislocation.

### Example 6

### Analysis of Minimum Inhibitory Concentration

Yptb bacterial cells were grown in 2XYT broth overnight at 26° C. Chloramphenicol (25 μg/ml) was added for bacterial strains containing a derivative of plasmid pACYC184. Bacteria were diluted in LB broth, and 96 well plates containing two-fold serial dilutions of N,N,N′,N′-tetramethylacridine-3,6-diamine (acridine orange), ethidium bromide, or pyocyanin were inoculated. Control wells lacked inhibitory agents. Some samples of bacteria were grown overnight at 37° C. without shaking, and absorbance was measured at a wavelength of 600 nm (OD600 or A₆₀₀) 18 hours later. The minimum inhibitory concentration (MIC) was calculated and is defined as the lowest concentration of toxic compound that results in half maximal growth (i.e. half the A₆₀₀ of the untreated control). Table 2 shows MIC values which are the average of six replicates.

### Example 7

### Characterization of Yersinia pseudotuberculosis (P⁻) Colonization of Mouse Organs and Determination of the Bottleneck Size

Plasmid-deficient strains of Yersinia pseudotuberculosis, Yptb (P⁻), grow and persist in various host organs (Balada-Llasat, J. M. et al. 2006 PLoS Pathog 2: e86, Simonet, M. et al. 1984 J Med Microbiol 18: 371-375). A genetic screen was performed for chromosomal Yptb virulence factors in mouse organs. It was previously believed that the number of clones that colonize the small intestine or successfully invade internal organs after oral inoculation was small (Mecsas, J. et al. 2001 Infect Immun 69: 2779-2787; Barnes, P. D. et al. 2006 J Exp Med 203: 1591-1601). Therefore, to increase the number of mutants that could be analyzed in a single mouse infection, ability of Yptb (P⁻) bacteria to infect mouse spleens and livers following intravenous injection was analyzed. It was observed that intravenous injection of 10⁵ Yptb (P⁻) bacteria into murine subjects resulted in presence of approximately 10% of the inoculum in the liver or spleen at four hours post-infection (FIG. 1 panel A). Furthermore, the Yptb (P⁻) bacteria present in these organs were able to survive for longer than a week, and exhibited roughly 30-fold greater growth in cell number during this time period of time (FIG. 1 panel A).

An estimate of the number of clones that colonized the spleen and liver was determined by calculating viable counts of bacteria in these organs at four hours post-infection, however it was unclear how many of these clones would survive the various aspects of the host immune system for a period of days. For example, the increase in colony forming units (CFU) in the liver from 10⁴ to more than 10⁵ between lour hours and three days post-infection could represent the loss of 99% of the clones, followed by 1.000-fold growth of each remaining bacterial clone. Alternatively, the increase in CFU could result from more than ten-fold increase in growth for each bacterial clone. Thus, the CFU values may be caused by a narrow bottleneck or wide bottleneck, respectively. This is a critical distinction in performing a genetic screen in an animal, as it determines how many mutants it is possible to screen in each animal.

Methods herein used transposon mutagenesis and deep sequencing (TnSeq), an insertion mutagenesis procedure that allowed calculation and monitoring of the presence of individual insertions by deep sequencing of the entire pool of insertion sites before and after inoculation. The TnSeq was used to determine the size of the bottleneck for Yptb(P⁻), the number of clones in the liver and spleen that initially entered these organs, and whether or to what extent the clonal number decreased over time (van Opijnen T. et al. 2009 Nat Methods 6: 767-772). Libraries of approximately 10⁴ mariner transposon mutants were generated. Without being limited by any particular theory or mechanism of action, it is here envisioned that this approximate number of mutants represents the maximum number of clones present in each organ as the protocol and method described herein resulted in no more than 10⁴ clones establishing residence in the liver or spleen.

Each of the pools of 10⁴ mutants was inoculated into mice, and bacteria were then isolated from the liver and spleen at time points of four hours, three days, or six days post-infection. Genomic DNA was isolated from the bacterial colonies obtained from these organs. Deep sequencing was then performed on the insertion sites to identify the number of clones that survived during this time period (van Opijnen T. et al. 2009 Nat Methods 6: 767-772). Data show the presence of about 7,600 clones in the liver and about 2,600 clones in the spleen, respectively (FIG. 1 panel B). A subsequent loss of clones was observed during the period of time, and the persistence of the vast majority of clones during a period of three days was surprising, as these plasmid-deficient bacteria lack many of the known virulence factors. By six days post-infection the number of clones in the liver detected was less than half the number determined to have initially colonized the organ, and the variance increased, as it was observed that some mice lost a greater number of clones than in others.

### Example 8

### Identification of Yptb(P⁻) Mutants Having a Phenotypic Defect in Colonization of Deep Tissue Sites

After obtaining the data from the clonal analysis (FIG. 1 panel B), mutant bacteria that were defective for growth/persistence in the liver three days after intravenous injection inoculation were sought by the screen, to maximize the number of mutant bacteria that could be screened and allow growth within these tissue sites. Each library of approximately 10,000 mutants was screened using ten murine subjects, for a total of more than 20,000 independent transposon insertion mutants, encompassing 3,088 genes (FIG. 2 panel A). The “output” samples for the screen were the pooled CFU from each individual infected liver, and the “input” samples were the pooled CFU from each library culture prior to inoculation by injection into subjects. The bacteria were scraped from the plate, and genomic DNA was isolated. The abundance of each transposon (In) insertion was quantified for each output and input sample using deep sequencing (van Opijnen T. et al. 2009 Nat Methods 6: 767-772).

Data obtained show that biological replicates of the input samples displayed very little variability as the binary logarithm (log2) of the ratio of each genus of biological replicates (BR1 and BR2) was about zero (FIG. 2 panel B), indicating the reproducibility of the method herein. Preparation of the input pool involved growth of the bacteria in culture at 26° C. prior to inoculation into subjects, so mutants defective for growth in subjects could simply have been temperature sensitive for growth. A control screen was performed by preparing a control pool of bacteria grown at 37° C. in culture to identify insertions that were depleted in the liver and also had general defects in growth at elevated temperatures. Data for the control screen were compared to the data for the input grown at 26° C. (Table 1 and Table 6).

Data for insertions in a given gene were then analyzed, and average ratios for output values and input values (output/input) were determined for each gene, using bacterial colonies isolated from each liver as a separate output and bacterial colonies isolated from the injection dose as input (FIG. 2 panel C). Genes of interest were identified as having a log2 normalized output/input ratio of ≧2.5 s.d. from the mean. The data from the screen are summarized in Table 1.

Insertion mutations that fulfilled the above criteria were grouped in four categories of genes, shown in Table 1. The categories were: genes encoding proteins required for disease in animal models (Known Virulence Factors; Mecsas, J. et al. 2001 Infect Immun 69: 2779-2787; Oyston, P. C. et al. 2000 Infect Immun 68: 3419-3425; Makoveichuk, E. et al. 2003 J Lipid Res 44: 320-330); genes encoding proteins involved in amino acid or nucleic acid synthesis; genes encoding proteins involved in LPS modification, especially O-antigen (O-Ag) synthesis; and uncharacterized genes or other genes encoding proteins not previously known to be important in previous Yersinia models of disease (candidate novel virulence factors).

Identification of five genes known to encode proteins implicated in virulence, including the genes encoding each of pH 6 antigen (mutations in 3 genes), invasin, and Sufi (Mecsas. J. et al. 2001 Infect Immun 69: 2779-2787), provided excellent positive controls for success of outcomes for the screen. Analysis of the data indicated that the screening used in Examples herein was able to mutate, isolate and thereby identify proteins that are important to virulence in a Yptb(P⁺) strain background (Table 1). Mutations in genes encoding proteins involved in amino acid and purine synthesis have been previously identified in screens for mutants defective for disease in animal models, and several orthologs of the genes identified in Table 1 are required for disease in related pathogens such as Salmonella enterica serovar Typhimurium (Davidson, A. L. et al 2008 Microbiol Mol Biol Rev 72: 317-364). The 14 genes required for LPS modification shown in Table 1, particularly those genes need for O-Ag synthesis, fell into two categories as well: those that are required for growth at elevated temperatures (e.g., 37° C.), and those that are not required for growth. For example, the genes that encode for the predicted O-Ag ligase (YPK_{—}3646; SEQ ID NO: 42) and WecA transferase (YPK_{—}4033; SEQ ID NO: 43) are both required for growth at 37° C. In contrast, a number of the genes that were predicted to be involved in LPS and O-Ag modification and synthesis were observed herein to not be required for growth at 37° C., and several have an intermediate, minor defect at 37° C. (Table 1).

### Example 9

### Identification and Characterization of Novel Yptb Virulence Factor, Mesenteric Lymph Node Required Transporter (mrtAB)

Of the mutations identified herein that were determined to be located in previously uncharacterized genes, insertions in two contiguous genes, YPK_{—}3222-3221 (SEQ ID NO: 1 and SEQ ID NO: 2 respectively) encoding a predicted heterodimeric ABC transporter (SEQ ID NO: 103) were observed to have the most severe predicted phenotypic defects. An in-frame deletion removing both genes in the plasmid-cured Yptb(P⁻) strain was generated to determine whether the defect predicted by the TnSeq analysis could be repeated during mouse infections using single strains. Three days after intravenous injection of the Yptb(P⁻) strain of bacteria into mice, it was observed that approximately 10²-10³ fewer bacteria were present in the liver and spleen, respectively, than in these organs injected with the parental Yptb bacteria (FIG. 3 panel A). Comparable data were obtained with individual deletions of the genes contained in the YPK_{—}3222-3221 operon (SEQ ID NO: 26). These genes in combination encode the heterodimeric transporter protein MrtAB (SEQ ID NO: 103). The presence of a lowered number of the mrtAB-deficient bacteria in deep tissue sites after intravenous inoculation of mice was not due to a general growth defect or to temperature sensitive growth, as the knockout strain of bacteria removing both genes YPK_{—}3221 and YPK_{—}3221 grew (i.e., mrtAB-deficient bacteria) identically to the parental Yptb(P⁻) bacteria in broth culture at 37° C. (FIG. 3 panel B). The defective splenic colonization phenotype of the YPK_{—}3222-3221 deletion mutant bacteria was almost completely complemented in a strain in which the two genes were placed on a low copy number plasmid pmrtAB in trans complementation. The number of bacteria in the spleen of subjects injected intravenously with the deletion mutant bacteria containing the complementing pmrtAB plasmid was similar to the number observed in the spleen of subjects injected with the wildtype bacteria carrying an empty vector as control (FIG. 3 panel C). The mrtAB-deficient bacteria survived transit through the blood and colonized the spleen or liver, as the number of bacteria (CFU) detected in these organs four hours after intravenous inoculation was identical to wildtype Yptb (P−) bacteria (FIG. 3 panel ID). Insertion mutations in the ABC transporter genes resulted in defective growth or persistence of bacteria in deep tissue sites, and in contrast did not cause an initial colonization defect or defective growth in culture for the bacteria.

The phenotype of the YPK_{—}3222-3221 deletion mutant in a Yptb(P⁺) background was then analyzed. Surprisingly, even though the absence of the predicted ABC transporter lowered yields of the plasmid deficient (P⁻) bacteria in the liver and spleen, there was no apparent defect in these organ sites after intravenous inoculation by a strain having the same mutation in the Yptb(P⁺) background (FIG. 4 panel A). Instead, deletion of YPK_{—}3222-3221 in Yptb (P⁺) bacteria resulted in a defect in colonization of only one organ, the mesenteric lymph nodes (MLN). Mice were orally inoculated with a wildtype Yptb(P⁺) strain or with the YPK_{—}3222-3221 deletion mutant strain. Results obtained show that bacteria lacking the putative ABC transporter were fully capable of persisting in the small intestine and of exponentially increasing their numbers in the Peyer's patches as rapidly as the wildtype strain. Yptb(P⁺) strains lacking the YPK_{—}3222-3221 ABC transporter displayed almost a 100-fold defect in the colonization or early growth in the MLN compared to wildtype bacteria (FIG. 4 panel B).

The observed defect in trans of the YPK_{—}3222-3221 deletion strain in colonizing the MLN was reversed by presence of a plasmid carrying both genes, demonstrating that the predicted ABC transporter genes were essential for a phenotype of early colonization of the MLN (FIG. 4 panel C). In addition, this early defect in colonizing the MLN was independent of route of administration, as mice intraperitoneally injected with the YPK_{—}3222-3221 deletion strain (i.e., mrtAB-deficient bacteria) were observed to have ten-fold fewer CFU in the MLN compared to mice injected with the wildtype Yptb bacteria (FIG. 4 panel D). Colonization in the spleen was observed to be comparable for wildtype Yptb bacteria and the YPK_{—}3222-3221 deletion strain (FIG. 4 panel D). As the wildtype cells lacking the mrtAB operon showed a specific defect in MLN colonization, operon (YPK_{—}3222-3221; SEQ ID NO: 26) encoding the MrtAB protein was named mrtAB, for Mesenteric lymph node Required Transporter.

### Example 10

### A Predicted ATP Binding Site of MrtB is Required for Survival In Vivo

The ATPase activity of ABC family transporters is a driving force behind the export or import of cargo across the membrane (Davidson, A. L. et al. 2008 Microbiol Mol Biol Rev 72: 317-364). A point mutation predicted herein to disrupt ATP binding in the MrtB Walker A box, was engineered on the pmrtAB complementation plasmid, to determine if the nucleotide binding site of MrtB was necessary for growth of Yptb in vivo. The corresponding amino acid change produced by the point mutation was predicted to disrupt the ATPase activity of ABC transporters (Davidson, A. L. et al. 1997 J Bacteriol 179: 5458-5464; Torres, C. et al. 2009 Biochim Biophys Acta 1788: 61 5-622).

The MrtB protein was engineered with a FLAG™ epitope tag (amino acid sequence DYKDDDDK; SEQ ID NO: 57) to determine if the predicted ATPase mutation would reduce steady state levels of the protein.

The ability of this mutated gene to rescue the growth of Yptb(P⁻)ΔmrtAB bacteria in the spleen was also determined by administering to mice the engineered strain. The observed CFU per spleen (FIG. 5, ordinate) is shown for mice intravenously administered 1×10⁵ three days earlier with each of either the Yptb(P⁻) strain, the Yptb(P⁻)ΔmrtAB strain, the Yptb(P⁻)ΔmrtAB/pmrtA⁺mrtB⁺-flag complementation strain, or the Yptb(P⁻)ΔmrtAB/pmrtA⁺mrtB*-flag strain carrying a complementation encoding a K380A mutation in MrtB. It was observed that mice receiving Yptb(P⁻) bacteria displayed a larger CFU compared to mice receiving Yptb(P⁻)ΔmrtAB bacteria (FIG. 5). FLAG-tagged MrtB on the plasmid encoding mrtAB (pmrtA⁺B⁺-flag) rescued growth of the ΔmrtAB bacteria in the spleen to the same extent as a wild type version of the gene (FIG. 3 panel C and FIG. 5 panel A). Disruption of MrtB Walker A box in the mrtAB flag complementation construct (pmrtA⁺B*-flag) resulted in a six-fold decrease in number of bacteria in the spleen three days post-injection (FIG. 5 panel A), without noticeably affecting protein expression (FIG. 5 panel B). These data show that the ATPase activity of MrtB is required for growth of Yptb(P⁻) bacteria in vivo.

To determine the effect of MrtA, a protein was engineered with a peptide tag hemagglutinin (HA) at the N terminus of the protein, located at the N terminus to avoid disruption of the mrtAB operon, in which the 3′ end of the mrtA gene coding region overlaps with the 5′ end of the mrtB gene. It was observed that mutation of the MrtA Walker A box had no negative effect on the rescue of Yptb (P⁻)ΔmrtAB bacteria by HA-mrtA*mrtB⁺ in the spleen. Further neither the HA-tagged MrtA protein nor the mutated HA-tagged MrtA protein was detected, possibly due to cleavage of both HA and with the signal sequence. Most important, data show that a binding site of MrtB is required for bacteria survival in vivo.

### Example 11

### Wildtype Yptb Cells Express YopE and are Associated with Neutrophils in the MLN

Analysis of growth and colonization data obtained herein indicated that the virulence plasmid bypassed a requirement for MrtAB in all organs except the MLN. After oral inoculation, yields of Yptb(P⁻) bacteria in the MLN are indistinguishable from a virulence plasmid-containing strain even though Yptb(P⁻) strains exhibit a growth defect in every other organ tested (Balada-Llasat, J. M. et al. 2006 PLoS Pathog 2: e86). Without being limited by any particular theory or mechanism of action, it is here envisioned that in the MLN, a large proportion of Yptb bacteria do not express the plasmid-encoded TTSS and Yops, making MrtAB essential for growth in this organ site. Further it is also possible the bacteria interact with different sets of innate immune cells in the MLN and the spleen, creating two distinct selective environments for Yptb cells in these organ sites.

A reporter strain was engineered herein in which the gene for the fluorescent mCherry protein was transcriptionally fused downstream from an intact yopE on the virulence plasmid, to determine whether Yptb strains do not express the plasmid-encoded TTSS and Yops. The yopE-mCherry construct was regulated in the same fashion as yopE during growth in broth culture, as cells encoding the fusion displayed thermally-induced mCherry expression that required the transcription factor LcrF (Garrity-Ryan, L. K. et al. 2010 Infect Immun 78: 4683-4690). It was observed that the mCherry protein was stably expressed, and that cells carrying the construct transferred from inducing to non-inducing culture conditions displayed reduced expression of mCherry protein (FIG. 6). Thus, the fusion yopE-mCherry protein is a useful tool for analyzing YopE expression at the single cell level in a replicating pool of bacteria that were detectable by constitutive GFP expression.

The expression of the YopE reporter was compared in spleens and MLN. Colonization of bacteria in the spleen after oral infection is known to occur much later than colonization in the MLN (Balada-Llasat, J. M. et al. 2006 PLoS Pathog 2: e86). Therefore, to approximately synchronize the wildtype yopE mCherry infections, colonization data was compared for spleens of mice intravenously injected two days earlier to colonization data for MLN from mice orally injected two days earlier. Surprisingly, it was observed that the expression of the YopE reporter was comparable in the spleen and MLN (FIG. 7 panels A-C).

TTSS secreted Yops proteins are preferentially found five days post-infection inside neutrophils in the Peyer's patches, MLN, and spleen, which may indicate an intimate interaction between Yptb and neutrophils during a late stage of the infection (Durand, E. A. et al. 2010 Cell Microbiol 12: 1064-1082). A possibility was tested, that at earlier stages post-inoculation, when there is a large difference in the requirement of MrtAB in the spleen compared to MLN, there would be altered co-localization of Yptb with neutrophils in these organ sites. Surprisingly, it was observed that two days post-infection the Yptb bacterial foci in the spleen and MLN displayed similar levels of co-localization with neutrophils, with 7/8 foci in the MLN, and 28/28 foci in the spleen strongly co-localizing with neutrophils (FIG. 7 panels D-E). The bacterial colonies in the MLN appeared to have a more diffuse morphology than the colonies in the spleen, however the respective colonies were still comparably co-localized with neutrophils. Without being limited by any particular theory or mechanism of action, it is here envisioned that wildtype Yptb bacteria express YopE and are associated with neutrophils in the MLN, and each of the Yptb colonies in the spleen and MLN contend with this potent innate immune cell.

### Example 12

### mrtAB Deficiency Results in Delayed Growth in the MLN, and Normal Spleen Colonization and Lethality During Oral Infection of Mice

The number of wildtype Yptb(P+) bacteria and mrtAB-deleted bacteria in the small intestine, Peyer's patches, and MLN of mice four days post-infection was analyzed to determine the role of MrtAB during a later stage of oral infection. Data observed herein show that MrtAB was not required for Yptb(P+) cells to persist in the small intestine or the PP following oral infection (FIG. 8 panel A).

Interestingly, while there were initially roughly five-fold fewer of the ΔmrtAB Yptb(P+) bacteria in the MLN compared to wildtype bacteria, the mutant strain after establishing itself in the organ were observed to have increased and largely caught up to the wildtype Yptb(P+) numbers in this organ by four days post-infection (compared to 100 fold fewer of the mutant strain one day post infection). Thus, the primary role of MrtAB was observed to have functioned during the initial colonization of the MLN rather than during later growth after the bacteria establish a replication site in this organ (FIG. 4 panel B, 8 panel A).

Ability of the mrtAB cells to colonize the spleen two days post-infection was analyzed to determine whether MrtAB was generally required for colonization of multiple organs following oral infection. The results obtained show that MrtAB appeared to be specifically required for MLN colonization, as the mutant colonized the spleen at a level comparable to wildtype Yptb(P+) control strain (FIG. 8 panel B). Further, it was observed that the wildtype Yptb(P+) strain and the mrtAB-deficient mutant strain caused an equivalent rate of lethality following acute Y. pseudotuberculosis oral infection with 10⁹ bacteria (FIG. 8 panel C). This result is consistent with the data that showed a similar ability of wildtype bacteria and MrtAB deficient Yptb(P+) bacteria to colonize internal organs such as the spleen. Thus the mrtAB deficiency resulted in delayed growth of bacteria in the MLN during oral infection, and resulted in normal spleen colonization and lethality during oral infection.

### Example 13

### Multicopy Expression of mrtAB Results in Enhanced Resistance to Ethidium Bromide and Increased Sensitivity to Pyocyanin

Little literature is available regarding the potential consequences of loss of MrtAB function. A phenotypic survey of E. coli genes revealed that a strain lacking the E. coli mrtB ortholog showed enhanced sensitivity to pyocyanin, an antimicrobial produced by Pseudomonas aeruginosa (Nichols, R. J. et al. 2011 Cell 144: 143-156).

No difference was detected herein in the minimal inhibitory concentration, MIC, of pyocyanin resulting from the absence of MrtAB, however an altered sensitivity under conditions predicted to overproduce MrtAB protein was observed. The vector used was a derivative of plasmid pACYC 184, which is a multi-copy plasmid, and expression of mrtAB on this plasmid resulted in increased susceptibility to pyocyanin. The point mutation predicted to interfere with the ATPase activities of MrtB protein removed this increased pyocyanin sensitivity (Table 2).

A further analysis was performed to determine whether there was an altered sensitivity for mrtAB strains to compounds that are substrates of efflux pumps. Ethidium bromide (EtBr) is a commonly used compound in the study of efflux pumps, as efflux provides the primary mechanism of EtBr resistance (Yu, E. W. et al. 2005 J Bacteriol 187: 6804-6815). Multi-copy expression of mrtAB enhanced Yptb resistance to EtBr, increasing the MIC by four-fold (Table 2).

The ability of MrtAB to confer enhanced resistance to EtBr strongly indicated that MrtAB functions as an efflux pump. Further, the ATPase function of MrtB was required for full EtBr resistance, in support of this model of MrtAB function (Table 2). The phenotype of increased resistance to EtBr and enhanced susceptibility to pyocyanin are indicative of a function of MrtAB export of substrates across the inner membrane into the periplasmic space, as the site of action of EtBr is in the bacterial cytoplasm, and that of pyocyanin may be in the periplasm (Baron, S. S. et al. 1989 Curr Microbiol 18: 223-230).

### Example 14

### Engineering of Antibodies Specific for MrtAB

Murine hybridomas secreting anti-MrtAB antibodies are generated using native MrtAB as an immunogen. The hybridoma supernatants are screened for their antigen binding capacity by enzyme-linked immunosorbent assay (ELISA) using microplates coated with MrtAB protein or portions thereof. Positive hybridomas are selected and cloned. The isotype of the monoclonal antibodies is determined by ELISA. Some of the antibodies are IgG isotypes, recognizing both native and recombinant mrtAB. The antibodies are observed not to cross-react to non-specific target proteins. The reactivity of the monoclonal antibodies are identified and mapped by Western blot and ELISA using full length MrtAB and truncated peptide fragments. Mouse IgG monoclonal antibody against an irrelevant antigen is used as an isotype control.

### Example 15

### Design and Construction of Inhibitory Sequences Directed Against Target Sequences

Methods are provided herein for targeting regions/domains of Yptb(P+) using siRNA or antisense RNA. A suitable region/stretch of coding mRNA for targeting by siRNA or antisense RNA is identified as having a number of properties including a secondary structure that does not appear to hinder silencing (Fire et al. 1998 Nature, 391:806-11; Tuschl et al. 1999 Genes Dev., 13:3191-7; Zamore et al. 2000 Cell, 101:25-33; Elbashir et al. Nature, 411:494-498; and Elbashir et al. 2001 Genes Dev., 15:188-200).

Target sequences in the nucleotide sequence of Yptb(P+) are identified and several siRNA sequences directed to each of polymorphic sequences and to non-polymorphic sequences in the genome of Yptb(P+) are designed. The regions that are targeted include portions of the Yptb(P+) genome regions within and surrounding the area identified as genes and operons, including for example YPK_{—}3221 (SEQ ID NO: 1), YPK_{—}3222 (SEQ ID NO: 2), and YPK_{—}3222-3221 (SEQ ID NO: 26) encoding a predicted heterodimeric ABC transporter. SiRNA agents/modulators are also developed using recombinant plasmid cloning methods, and sequencing the vector to confirm successful insertion. A portion of the siRNA is engineered operably linked with a green fluorescent protein (GFP) reporter. A number of siRNA vectors are engineered with multiple expressions cassettes.

### Example 16

### In Vitro Modulation of Virulence Factors

Examples herein used the agents prepared herein (i.e., antibodies and sirRNA) to determine whether the activity and cytotoxicity of Yptb(P+) bacterial strains could be modulated. Agents are prepared herein, which are antibodies and siRNA to target mrtAB expression and/or the MrtAB protein or a portion thereof.

The Yptb(P+) bacterial cells are contacted with siRNA-encoding vectors and inhibition of expression of mrtAB in cells is analyzed. The vector preparation is incubated with the target cell population in culture for hours and overnight at different multiplicities of infection (MOI) between 0.1 and 10, and the medium is replenished the following day. After two to three days, viral transgene expression is confirmed by flow cytometric detection of the linked GFP reporter.

Further samples of cells not contacted with siRNA-encoding vectors are induced by growth in cell culture and monoclonal antibodies of different concentrations are administered to the culture and to cell lysates.

It is observed also that contacting Yptb(P+) cells with vectors carrying siRNA that target mrtAB operon decreases the expression of markers indicative of a Type III Secretion System (TTSS) such as Yops. Further data show that monoclonal antibodies that specifically target mrtAB protein on the cell membrane also reduce expression of the markers. Therefore, the in vitro data show that agents/modulators described herein inhibited the virulence factors in Yptb(P+) cells, and inhibition using siRNA or binding of MrtAB protein on the cytoplasmic membrane using immunoglobulins is associated with decreasing of expression of mrtAB operon.

### Example 17

### In Vivo Modulation of Virulence Factors

To determine whether the activity and cytotoxicity of Yptb(P+) could be modulated in vivo, animal subjects are infected with Yptb(P+) bacterial cells. Blood and tissue samples are collected at various time points, and are analyzed for markers of Yptb(P+) infection. Subjects are orally or intravenously administered with either vectors that carry siRNA that negatively target expression of mrtAB, or are administered immunoglobulin preparations that specifically bind MrtAB protein. Control subjects are untreated, administered buffer only and neither agent or are administered control siRNA or non-specific immunoglobulins. Blood and tissues samples are collected at the time points and analyzed for the markers. Data show longer survival and decreased presence of infection markers in samples for subjects administered vectors carrying siRNA specific for mrtAB genes and for subjects administered anti-MrtAB immunoglobulins compared to control subjects administered control siRNA or non-specific immunoglobulins. It is also observed that the extent of the decrease is in many cases a function of the number of vectors carrying the vectors carrying siRNA specific for mrtAB gene, and concentration dependent of anti-MrtAB immunoglobulins.

What is claimed is:

1. A pharmaceutical composition for treating or preventing a disease associated with a Gram-negative bacterial strain, the composition comprising a modulator of a virulence factor, wherein the virulence factor comprises a protein, wherein the modulator specifically binds to the virulence factor, and inhibits function or binds to a gene encoding expression of the virulence factor, wherein the composition prevents the Gram-negative bacterial cell growth and infection in tissues of the subject.

2. The composition according to claim 1, wherein the modulator comprises a nucleic acid vector, wherein the vector comprises DNA, mRNA, tRNA, rRNA, siRNA, RNAi, miRNA, or dsRNA.

3. The composition according to claim 1, wherein the modulator comprises at least one selected from the group of a protein, an antibody, an enzyme, a carbohydrate, a sugar, and a small molecule.

4. The composition according to claim 1, wherein the modulator comprises a nucleic acid binding protein that inhibits a gene encoding the virulence factor.

5. The composition according to claim 1, wherein the virulence factor protein is at least one selected from the group of: a transporter, a mesenteric lymph node required transporter (MrtAB), a lipopolysaccharide synthetase, a pH6 antigen, an invasin, an Ail, a flagellin, an attachment and effacement regulator, a cytoskeletal protein, and RodZ.

6. The composition according to claim 1, wherein the virulence factor is encoded by a nucleotide sequence of at least one gene selected from the group of YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof.

7. A method for treating or preventing a disease associated with a Gram-negative bacterial strain in a subject, the method comprising contacting a tissue of the subject with a composition comprising a modulator of a virulence factor identified by mutagenizing cells of the strain and isolating mutated virulence factors, wherein the modulator is specific to bind to the virulence factor to inhibit function or to bind to a gene encoding expression of the virulence factor, wherein the composition prevents the Gram-negative bacterial cell growth and infection in the tissue of the subject.

8. The method according to claim 7, wherein contacting the cells or the tissue of the subject with the modulator comprises delivering a nucleic acid vector that inhibits expression of the virulence factor comprising a protein selected from the group of: a transporter, a mesenteric lymph node required transporter (MrtAB), a lipopolysaccharide synthetase, a pH6 antigen, an invasin, an Ail, a flagellin, a cytoskeletal protein, and RodZ.

9. The method according to claim 7, wherein the gene comprises a nucleic acid vector including DNA or RNA, wherein the nucleic acid vector comprises a genetically engineered genome derived from at least one virus selected from the group of: adenovirus, adeno-associated virus, herpesvirus, and lentivirus.

10. The method according to any of claim 7, wherein prior to contacting, the method involves engineering the modulator by constructing at least one of: mRNA, tRNA, rRNA, siRNA, RNAi, miRNA, and dsRNA, or a portion thereof.

11. The method according to claim 7, wherein contacting comprising administering the modulator to the tissue selected from: muscular, epithelial, endothelial, lymph, and vascular, wherein the tissue is in at least one of: eye, heart, kidney, thyroid, brain, stomach, lung, liver, pancreas, stomach, liver, spleen, pancreas, and gall bladder.

12. The method according to claim 7, wherein the modulator comprises at least one selected from the group of: an antibody, an enzyme, a nucleic acid binding protein, and a fusion protein.

13. The method according to claim 7, wherein contacting the cells comprises contacting the tissue in situ or in vivo, wherein the cells are at least one selected from the group consisting of: muscular, epithelial, endothelial, vascular, eye, heart, kidney, thyroid, brain, abdomen, stomach, gastrointestinal tract, lung, liver, pancreas, spleen, and lymph node.

14. The method according to claim 7, wherein contacting the tissue comprises adding the modulator to the tissue ex vivo to form a mixture, and then administering the mixture to the subject.

15. The method according to claim, wherein the nucleic acid vector encoding the modulator inhibits the virulence factor encoded by at least one gene shown in Table 1 and is selected from the group of: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof.

16. A method of identifying a therapeutic agent for treating or preventing a disease in a subject associated with a Gram-negative bacterial strain, the method comprising:
contacting a first sample of cells or tissue with the strain expressing a virulence factor, contacting a second sample of the cells or tissue with the strain and the therapeutic agent, and contacting a third sample of the cells or tissue with the strain and a control agent encoding a detectable protein that does not induce the colonization of the cells or the tissue, wherein the first sample, second sample, and third sample are each from the subject; and
measuring an amount of the marker in the first sample, the second sample, and the third sample, wherein the marker is characteristic of the disease, wherein the increased amount of the marker in the first sample compared to the second sample is a measure of treatment and protection by the therapeutic agent, wherein a decreased amount of the marker in the third sample compared to the first sample is an indication that the agent is therapeutic, thereby identifying the potential therapeutic agent for treating or preventing the disease.

17. The method according to claim 16, wherein the Gram-negative bacterial strain is a short facultatively aerobic or micro-aerobic rod or an enteric strain.

18. The method according to claim 16, wherein the detectable protein is at least one selected from the group consisting of: a purification tag, a fluorescent protein, an enzyme, a colorimetric molecule, a chemifluorescent protein.

19. The method according to claim 16, wherein the therapeutic agent is selected from the group of: a vector, a viral vector, a nucleic acid, a DNA, a RNA, a protein, an enzyme, an antibody, a small molecule, a carbohydrate, and a sugar.

20. The method according to claim 16, wherein measuring the marker comprises detecting presence, activity, or amount of a protein or a nucleic acid.

21. The method according to claim 16, wherein contacting comprises contacting the first sample, second sample and third sample in an animal model in vivo or in vitro, wherein the cells or the tissue comprise at least one selected from: muscular, epithelial, endothelial, vascular, eye, heart, kidney, thyroid, brain, abdomen, stomach, gastrointestinal tract, lung, liver, pancreas, spleen, and lymph node.

22. The method according to claim 16, wherein measuring further comprises observing at least one of: cellular morphology, cell viability, cellular pathology, and tissue pathology.

23. A kit for modulating growth or severity of a disease associated with a Gram-negative bacterial strain, the kit comprising:
a modulator of a virulence factor expressed by the Gram-negative bacteria, wherein the virulence factor comprises a protein, wherein the modulator is specific to bind to the virulence factor to inhibit function or to bind to a gene encoding expression of the virulence factor, wherein the composition prevents the Gram-negative bacterial cell growth and infection in tissues of the subject;
instructions for use; and,
a container.

24. The kit according to claim 23, wherein the gene comprises a nucleic acid vector including DNA or a RNA, wherein the nucleic acid vector comprises mRNA, tRNA, rRNA, siRNA, RNAi, miRNA, and dsRNA, or a portion thereof.

25. The kit according to claim 23, wherein the modulator comprises at least one protein selected from the group of: an antibody, an enzyme, a fusion protein, and a nucleic acid binding protein.

26. The kit according to claim 23, wherein at least one gene encoding the virulence factor is at least one selected from the group of: YPK_{—}3221 (SEQ ID NO: 1), YPK_{—}3222 (SEQ ID NO: 2), YPK_{—}1234 (SEQ ID NO: 3), YPK_{—}2423 (SEQ ID NO: 4), YPK_{—}1292 (SEQ ID NO: 5), YPK_{—}2066 (SEQ ID NO: 6), YPK_{—}3575 (SEQ ID NO: 7), YPK_{—}1713 (SEQ ID NO: 8), YPK_{—}2406 (SEQ ID NO: 9), YPK_{—}3656 (SEQ ID NO: 10), YPK_{—}0453 (SEQ ID NO: 11), YPK_{—}0688 (SEQ ID NO: 12), YPK_{—}2424 (SEQ ID NO: 13), YPK_{—}3600 (SEQ ID NO: 14), YPK_{—}2199 (SEQ ID NO: 15), YPK_{—}4078 (SEQ ID NO: 16), YPK_{—}0208 (SEQ ID NO: 17), and a portion thereof.

27. A pharmaceutical composition for treating or preventing a disease associated with a Gram-negative bacterial strain, wherein the composition specifically binds to a protein virulence factor to inhibit its function or to a gene encoding expression of the virulence factor to inhibit its expression, and prevents the Gram-negative bacterial cell growth and infection in tissues of the subject.


SEQUENCE LISTING (103 sequences)

SEQ ID NO: 1 - DNA, 1824 bp, Yersinia pseudotuberculosis
atgaataacgctcagcaactttggccgacattgaaacgcctgctttcctacggttcgccttatcgtaaaccgctggggctggcagtcttaatgctgtgggttgcggcagcagcagaggtaagtggtccactactgatcagttattttattgaccatgtagtcgccaaaggcacattacctctggggctagtcagtgggttggcattggcctatctgttgttgcaattattggccgcgacattgcactattttcaggcattgctgtttaaccgtgcggcagtgggggtcgttcagcggctacgcattgatgtgatggacgctgcattacgccaacctctcagtgcttttgatactcagcctgttgggcagttgatttcccgagtaaccaatgacactgaagtgatcaaagatttatatgtcatggtggtttctacggttttgaaaagtgcggccttaattagtgcgatgctggtggcgatgtttagtctggattggcggatggcgctgatttcaatttgtatcttccctgcggtgttggtggtgatgacaatctatcagcgctatagcacccctatcgttcgccgggtacgatcctatctggctgatattaacgatggttttaatgaagtcattaatggcatgggcgtcattcagcaattccgtcagcaggcccgtttcggggaacgcatggcgtctgccagccgtgcgcattatgttgcccggatgcaaaccttacggctggagggctttctattacgccctttattgagcctattctccgccttggtgttatgtggtttactgctgcttttcggtttcagccctgaaggttctgtgggggtgggtgtgctgtatgcgttcattaattaccttggccgcctgaatgagccgttgattgaactgacatcgcagcaatcaattatgcaacaggccgtggtggcaggagagcgtatttttgatctgatggatcgtgcgcagcaggactatggtagcgacaatattcccttgagcagtggccgtattcaggtggaaaacgtcagctttgcgtatcgctcggataaaatggtgttacacaatatttctctcagtgtcccctcccgtggatttgtggctttagttgggcacaccggcagtggtaaaagtacgctagctaacctgttgatgggctactatcccattcagcaaggtgagattttgcttgatggccgaccattatcccgcctctcccatcaggttctgcgccagggggtagcgttggtacagcaagatcccgtggtgttggctgactccttctttgcgaatatcacccttgggcgtgaccttagtgaacagcaagtgtgggaggcactcgaaaccgttcaattggcaccgttggttcgtaccttacctgatggcttgtacagtttacttggggaacagggcaataccttgtctgttgggcaaaaacagttgctggcgatggcgcgggtgttggtgcaagcgccccaaatcctgattctggatgaagccactgcgaatattgactctggcactgaacaggctattcagcgggcattacaggtgattcgaaaaaataccacgctggtggttattgctcatcgcctttcgacgattgtggaggcggatagtattttggtactgcaccgtggtgttgcggttgagcagggtaatcatcaagcgctgctagctgcccgtgggcgttattaccagatgtaccagttgcagttagtcagcgaagatttggctgccattgatcaggaagctattgataaaggctctattgatcaaagcactattgatcaagccggaatgagcgtgagttaa

SEQ ID NO: 2 - DNA, 1767 bp, Yersinia pseudotuberculosis
gtgagattgtttgcacaattaggttggtattttcgccgtgaatggcaccgttatgtaggggcggtgctactactgattattatcgctattctgcaattaattccgcccaagttggtgggcgttatagtggatggtatcagtacaaaacagatgtccaccaatatgttattggtttggattggcgtgatgctcgcgactgccgtggtggtctatttgttgcgttatgtctggcgagtcttattattcggtgcctcttatcagttggcggtggagctgagatctgatttttatcgtcagctgagtcggcaaactcccggtttttattcacgtcatcgcactggcgatttaatggctcgcgccaccaatgatgttgatcgcgttgtcttcgccgcaggtgaaggggtactaacgctggtcgattcactggtcatggggtgtgcggtattgattgttatgagcacccaaatcagctggcaattaacactgttatcgctattaccgatgcccatcatggcgatagtgattaagtattacggtgaccaacttcatcagcgttttaaatccgctcagggtgccttttctttgcttaataatcaggctcaagagagcctgaccagtattcgcatgattaaggcctttggtctggaagatcgtcagtcacaacagtttgctcaggtagcggtagaaaccggtgcgaagaatatgtatgtcgcccgcattgatgcccgctttgaccctacaatttatattgctattggtatcgctaatttattagctattggtggcggtagctggatggtggtgaataacagcattaccttagggcaattaaccagttttgttatgtatttggggctgatgatttggccaatgctggcattggcatggatgtttaatattgttgagcgcggtagtgccgcttatagccgcatccgcagtttgttggatgaagcacctgtggttaaagatggccacattactttgtctgatgtgcgtgacacattagcggttaatattcgtcatttttgttatccaggcagtgatcaaccggcactacataatgtggtactgacgctggttccaggggccatgctggggttatgtgggccaacaggttcgggtaagagtaccttgctggccttaatccagcggcaatttgatattgatgacggtgttatttgttatcaagggcatccgctgtcggatattcggttgaatgattggcgtggccgtttatcggtcgttagccagacccctttcctgttctccgattcggtggccggtaatattgccttgggtaaacccgatgcgacccccgcacagattgagcaggcggcccgcctggcctgtgtacatgaagatattttgcgtctgccgcagggttatgacactgaagtgggcgagcgtggagtgatgttgtccggtggtcagaaacagcgcatatctattgccagagcgcttttgttggatgctgaaatacttattcttgatgatgcactttctgcggttgatggtcagacggagcatgaaattttaaaaaacctgcgcgagtggggcgaacaacgtaccgtcattatcagcgcgcatcgcctttctgcattgaccgaagccagcgaaattctggtgatgcaacatggcggtgtaatgcagcgggggccccacagccttttagtcaatcagacgggttggtatcgggagatgtatcgctatcagcaattagaagccgcattggatgacggtgagcaggaggtcgaagccgatgaataa

SEQ ID NO: 3 - DNA, 807 bp, Yersinia pseudotuberculosis
atgctgagatttgaccaatttgctattgatgtcgctcattatcgttggttgggccggaaaacctggcacccgttgttgagccatatttctctggatgtgaatccgggggaattggtggcattggtcggcgggagtggagagggtaagagcttgttactccaaagtgtcttgggtttattgccggagaatatgcggtgccatggcgagattatcctcgatggtgaggtactgtgcgcccgcgataaaattgagcagcggggtaaaaccttatgttatgtcccccaaggcgtcagtgctctgaacccactgattaaagttggctcacaaatcacccgagcagcgcaactcagtggtgtaaaactgcgtttggaagatgtcgccatgcagttgaaaacttataatcttgcgccggggctggtaaatgatttcccgcgtcagctctcaggcggcatggcgaaacgggtactgaccagctgcgcgaccttgacccatgcccgctatattctggccgatgaagtgacctcatggctagatgaagaccatgcttgccagttactgacgcatctgcgtgctttttgtcagcaggggcgcggtattctgtgggtaacccatgatttagcgttggccgcacgttttgccgataaaattgcggtgttacataaaggtgagttgcacgaaacattgagcgctgacgaactgaaaaatagcggtggtagcccatggttgcagtcactctgggccgcattaccggaacagcaatttgtcagcaagccaatcccactggctgaggtggccgactatgcttag

SEQ ID NO: 4 - DNA, 687 bp, Yersinia pseudotuberculosis
atggccattaccaattccctgacggaaagtgattacggtattaccggcacgaccgggaccagttccactaccggcagcagtagtcaagatctgcaaaacagcttcctgacgttattagtggcgcagttgaaaaaccaagaccctactaacccaatggaaaacaacgagttgaccacacagttggcgcaaatcaacaccgtgagtggcattgaaaagctgaatacgaccctcggtgctattaccggtcaaattgacagcagccaatctctgtacgccaccagcctgattggtcgaggcgttatggtccctggtaccaatatatttacgggcagcaccgatggcacggtcagcaccacgccttttggcctcgaattgcaacgacctgcggataaagtgaccgccaccattagcgatagcaatggtcaggtggtacggaccattgagatcggtggtttaaacgcaggtgttcactcctttacctgggatggctcactggacgcgggcggtaatgcgccagatggtgcttataccgtagccattactgccagcaatggtggggaatcactggtcgcaacgccgttgaactacgccattgtcaacggtgtaacccgcggcgcggatggctccaaactggacttgggtctggcaggaaccatcacgctggatgaagtgcgccagattttataa

SEQ ID NO: 5 - DNA, 1107 bp, Yersinia pseudotuberculosis
atgaatactgaagcctcccaagatcaaactgttacagagacgccaggtgtgcgcctgcgtcaagcccgcgaatcattagggctaacccagcagactgttgcagaacgtctgtgtctgaaagtatccacaatccgtgatattgaggaggataacgcgcaagctaatctcgcctcgacattccaccgtggctacattcgttcttacgccaagctggttcatcttcctgaagatgagttattgcctattttggagaaacaagcgccagtcagagcggcgaaagttgccccaatgcaaagtttttcattgggtaaaaaacacaaaaaacgtgatggctggttgatgagtttcacctggcttattgtgttggttgtgttaggtctgaccggagcatggtggtggcaaaatcatcaggcacagcaagcagagattgccaatatggttgatcaatcttctgcccaactgtcacaaaatggcgggcaaccagtgccgctaactgatgataacagcgatgctattgcgccaacggatgccccggcaccggtagccaatggtcagccggtaccactgacaaatcattcaacttcggcagtaactaattcagctacaaccagttcagctacaaccagttcagttccaacaacaagttcagttccaaaaacaactttagttccaaaaacaactttagttccaaaaacaaatagtactgaacctgtcgatacggcgaatacaaataccacaatgcatcaggagggggctgcgtcagcggccgtttccccaagtcaggtaccgcaacttggtatgcccacagaccaaccgccattacctacagcagacgctggggtaagtggtagcgcgtcatctgtgggggcgttggtgatgaattttacagcagattgttggttgcaggttgttgatgcaacgggcaagacgctgtttagtggtattcaaaaagggggcgctgtgcttaacctggcaggtaaagcgccctataagctgactatcggtgcaccaggtgctctgacgatttcgtatcagggtaacccggtagatttaagtaagtttattaaggctaatcgcgtagcccgcctgactgtcggtgtagagtag

SEQ ID NO: 6 - DNA, 324 bp, Yersinia pseudotuberculosis
ttgagaaaaaacgtgaaatatttgctgatttttttattagtactggtggttttcgtgatttcagtcacgttaggggccaataacgaccaactcgtcgcctttaactatttactcgctcaaggtgaatatcagctgtctacgctattggcaacgctttttgcggccgggctggtgctaggctgggtcatttgtgggcttttttatctgcgggtacggatctctttgggccgcgcagagcggaaaattaaacgtattgaaactcagcttgaacagcctgttgagcaaagcacgcttgtgtccacagctgctgtcaacaaggaataa

SEQ ID NO: 7 - DNA, 870 bp, Yersinia pseudotuberculosis
atgtctacttatcttattggtgatattcatggctgcctcgatgaattacttgcgcttttagcccaggtgaacttcgatccacagcaggatactttatggctgacgggcgatttagttgcccgtggtcccgcttcattggatgttttacgttatgtacgttcgttaggacccgcagttcgtatggtgctgggtaaccatgacctgcatttactggcagtctacgcaggcatcagccgtaataaaccaaaagatcgtattaccccactgctggatgcacccgatgctgatgagctaattaattggctacgtcgccaacccgttttgcaggttgatgaccagcttaaattgattatggcccacgcaggcatcacaccacagtgggatattgaaacagcccaaatgtgtgcgcgagaagttgaagcggtattgagcagcgacagttatccgctatttttggatgcgatgtacggtgatatgccaaacaactggtcaccagagcttaccgggctggcacgtttgcgctttagtaccaatgcacttacgcggatgcgcttttgcttcccgaatggccaacttgatatgatctgcaaagacacgccagaaaacgcgccagcccctctcaaaccttggtttgacctaccaaggctcgttgaccctgaatattcaattatttttggtcattgggcatcattggaaggtaaaggtgtaccagaggggatatatggactggacactggctgttgctggggaggggatttaaccctacttcgttgggaagataaacgttatttcacacagcgcgccttcaaagccgaagctgaaattaataataacaatggatttgcagcaggcgaagaagtacaacactaa

SEQ ID NO: 8 - DNA, 1122 bp, Yersinia pseudotuberculosis
atggattaccaactcgatctcgattggcctgactttctacaacgctattggcaaaagcgccctgttatcctcaaacgtggcttcaaaaattttattgacccactctcaccagatgaactggccgggttagccatggaaaatgaagtcgatagccgcctggtaagccatgaagatgggcgttggcatgttagccacgggccatttgaaagcttcgatcatttaggtgaaaacaactggtcattgttagttcaggcggtagaccattggcatgaacccgcggcggcgctaatgcgccctttccgtccactctctgactggcgtatggatgatttaatgatctccttctccgtgcctggcggtggtgttgggcctcattttgatcaatatgatgtttttattattcagggttcaggccgtcgccgctggcgggtgggcgaaaaaactgaaatgaaacaacattgcccgcacccagatttgctccaagtggggcctttcgacgctatcattgatgaagaaatggagcctggtgacattctttatattccaccgggcttccctcatgaaggctattcccttgaaaatgcgctgaattactccgttggtttccgcgccccaagtggtcgagaactggtcagtggttttgcggattatgtattggctcgagaactgggtagctatcgttatagcgatccagatttacagctacgcgagcatccagccgaagtattaccgtccgaagttgataaactgcgcacaatgatgctggatctggtccagcaacctgaacatttccaaaactggtttggtgaatttatttcccaatcacgccatgagttggatcttgcaccgccggagccgccttatcagaccggcgatatctatgaactattgaagcaaggcgatgaattacaacgccttagtggattacgggttctgcgggttggtgatcgttgctttgctaatggtgagttgattgatacgccacacttacaggccgccaatgcactgtgccagcactttagcgtgaatgcagagatgttgggtgatgcacttgaagacccttctttcctggcaatgcttgcagcactggtcaatagcggttattggtattttaacgattga

SEQ ID NO: 9 - DNA, 600 bp, Yersinia pseudotuberculosis
atgtacttatcaaaaccaagtatcatatatggtttccatggaatggatgaagatgcagcattacccattctattaaaaaaagacaacttcaaacacagtaataattcatatgattggcttggcaatggtgtttatttttgggaaaacaattatgaaagagccattcaatatgctattgaagataaagcaagaaaaaactcgagaataaaaaaaccatttgtattaggcgctattattgaccttggtaattgtttagacttatttgatcaaaagcatattgattttctgaaattctcatttgaagaaatgcttggatcattaaattcgcaaaagaaagaaataccggtgaataaaaaatttggtaacagcgattttgattttagatgtagagaattagattgtgcggttattcgatatgcccacactcttgcaaaaaaagaaggaaaaccattcgactctgttagagccgcttttttagaaggtaatcctttgtatgaaggcgctaaattttatgaaaaaaaccatatccaaatcgctgtactaaatcccaactgtattaaaggggtcttttacccaagggaaaaagtagtttacccttaa

SEQ ID NO: 10 - DNA, 492 bp, Yersinia pseudotuberculosis
atggataacattcatcgcctattcttagcactggctttttctgccccgttggtgaccggtgctgctcacgccaacagtttattggattcggtaaaaagcaccgcggagcaatacggtaaatcagccggtagctcatcgtcactgccgtcgatgtcatcactgacgaacttactcaatggcggagataaagcgttaagtgcaggcaccatgaacaatgccaccggtattttgcaatattgcgtacagaataatgtgttatcaagcgatggcaccacggcgattaaagaccaactgatgagcaaactgggtatcagtggtacagaagcggcgaaaagtacggattatgaagagggcttgggcgggttgctgaaaaccagtgaaggcaaaagcgtaaacctgaatgacctcggtaccgggcaaatcacagaaaaaatcaaaaccaaagcctgcgatttagtgcttaagcaaggaaagtcattcttattgaagtaa

SEQ ID NO: 11 - DNA, 966 bp, Yersinia pseudotuberculosis
atgcgtattggacacttccagcttacaaattgcctgattgccgccccgatggcgggtatcacagatcgccccttcagagcgctatgtcatggtatgggggctgggatggctgtatctgaaatgctctcctctaatccagaggtttggcgaacggataagtcgcgtttacgcatggtccatgttgatgaacccgggattcgtaacgtgcaaattgccggtaacgatccagatgagatggcagcagccgccagaatcaatgtggcgagtggtgcccaaatcatcgatatcaatatgggatgtccagccaagaaagtgaaccgcaaactggcgggttctgcattattgcagcatcctgatctggtcaaacagatcctctccgcagtggttaatgcggtagatgtgccggtgacgttgaagatccggacgggttggtcaccagaacaccgtaactgtatagaaattgcccaactggccgaaaattgtggtatacaagccctgacgattcatggccgaacccgttcttgcctgtttaatggtgaggcggaatacgacagtattcgagcagttaagcagactgtttccattcccgttatcgcgaatggtgacatcactgacccgcataaagccagagcggtactcgactacactggagctgatgctctgatgataggccgtgccgctcagggaagaccgtggatcttccgggaaatccagcattatctggacactggggaactgctgccaccgatgccacttggcgaagtgcagcgtttgttagacgggcatatacgggaattgcacgacttttatggtccaggcaagggatttcgtattgcacgtaagcacgtctcttggtatctccaagagcacgcccctaacgaccagtttcggcgcacattcaacgccattgaggatgccagcgaacagctggaggcgttggaggcatatttcgaaaatcttgcgtaa

SEQ ID NO: 12 - DNA, 198 bp, Yersinia pseudotuberculosis
atgatgaacttcatgactaaagcttacgttaccgcacaagttaaagcacaagccttcgcacaggataaccgtggttctatcattgaatatgtgatgatcattgcactggcgggtgtgtttattggtctggcaaaaccagaactgacagacatcatcaccgataccgttgccaaaacaaaagcagccattgctccataa

SEQ ID NO: 13 - DNA, 405 bp, Yersinia pseudotuberculosis
atgtctttactgaatatttttgatattgccggttcagcattatcagcccagtcgcaacggatgaacgtcagtgccagtaatctggccaacgccgacagtgtgacgggccccgatgggcaaccttaccgcgccaagcaagtggtcttccaggtggctgcgcagccagggcaggaaaccggtggggtgcgggttgctcagatagttgatgatccgtcaccggatcgtttggtgtatcagccgggtaacccgctggcggatgccaaagggtatgtgcggatgccgaacgttgatgtgacgggggaaatggtcaacaccatttctgcatcacgtagctaccaggcaaacgttgaagtcctgaataccaccaaatccctgatgctcaaaacgctgacactgggtcaataa

SEQ ID NO: 14 - DNA, 777 bp, Yersinia pseudotuberculosis
atgcttattattatctccccggccaaaacgcttgattatcaaagccccttagcgacaacgaaatttagccaaccagaaatgttggataaatcacaagcattgattgagatatgccgcgagctaaccccggctcaaatcagcagcctgatggggattagcgataaactggcaggtttaaatgccgctcgattcagtgaatggcagcctgatttcacacccgcaaatgcgcgtcaggctattctggcttttaaaggtgacgtctataccgggatgcaggccgaaagtttcagtgaagctgattttgactttgcacaacagcatttgcgtatgttgtcaggtttatacggtctgctgcgcccgctagatttaatgcaaccttatcgtttagaaatggggactaagctggctaacccacgcggcaaagatctttatgccttttggggcgatcagataaccgagaaacttaatcaggcgctggaactgcaaggcgataatattctgattaatttggcgtccgatgagtattttaaggctgttaaacccgctaaattaagcggttcactgattaaaccggtatttttggatgagaaaaacggtaagtacaaaataatcagtttctacgcgaagaaagcccgtggcctgatgagccgctttattattcagaataagttaaccaaaccagagcaattggttgattttaatctggaaggctatgaatttgatgcgggattatcggcgaaaaacgaactcgtgtttaaacgggctgaacagcactaa

SEQ ID NO: 15 - DNA, 951 bp, Yersinia pseudotuberculosis
atgctcctaaataatattacgccagtgaataaatcactgacactacaagatttattaggaattttgagtcactcatcggcaatttccaatgtggcaaatgggatttatgtggaaagcgaaatccttgaagtaggttcatggctttcagcctacgcggctaataaagatgaaattttttcgcagatcattaccgagttggagaacccttatcaattccagctggagaatgacatacaggcaccgagttttattctttacagtaatgaacgcataactattcgtcttgttatgtggctcccattgcagggaaaattagatcggacaccttattcctacgaagaagcacatgatcataattttgacttttggacagtgaatttttttggaggtggctatcgaactcggctctatgactatgattacgataaagttagtggcgtaaataacgaagttgtagaacttaattgttatggagataagattctttccccgaatacagttatgttttattttcgtagtaaggatgttcatactcaatatccgcctgatgaattatcagtatcgctcaatttaatagtgcgaccaataaaatcgaagcatcaatacgaatttcagatagattcagatgcattggaaggaaaaatagaggcaagaattaaaaagggaagatatgagcgctacgcttttcaaaatgtgttatataacggcctactgagtcttgaaaatgaaaaaagtcgtcaactggttcacaaagtgtctctttgtaaccatcgagaagagatacgattaatcgcttatgaagctttacttaagcacgcccaaaaaaaaggtaatgtgagtgatataaagagcattagtgaacaagcatttaaagaccaaagcctctatatcaaaaataagatttctcacagtattggaagcatgccatgcatgagtccaaaaccccgctaa

SEQ ID NO: 16 - DNA, 1401 bp, Yersinia pseudotuberculosis
atgcaacagcacttccattttgatgccattgtgattggctcgggccctggcggtgaaggtgccgcgatggggctggtcaagcaaggggcacgcgttgccgtgattgaacggtataataatgtgggtggcggatgtacccattggggtaccattccttctaaagccttgcgccacgccgttagccgtattatcgagttcaaccaaaaccccctctacagcgacaatgccagaaccataaaatcctctttcgctgatattttaaaccatgcagatcgtgttattaatcagcaaacccgtatgcggcaaggcttttatgatcgcaaccattgccacatgttctcaggtgatgccagcttcattgatgccaatacggttaatgtgcgttacgccgacggtaccagtgataccttgcaggccgataacattgtcattgcaactggctcgcgcccttatcgcccagtcaatgttgattttaaccatgaacgcatttatgacagcgataccattttgcagctcagccatgagccacagcacgtcattatttatggtgcaggggtgattggttgcgaatatgcgtctatcttccgtgggttgagtgtcaaagtcgatttgatcaatacccgtgaccgcctactggctttccttgatcaggaaatgtcagatgcgctctcttatcacttctggaataacggcgtggttatccgccataacgaagaatttgagcagattgaagggacaacggacggcgtgattgtccatttgaaatcaggcaaaaaggtcaaagcagactgcttgttatatgctaatggacgaacaggtaataccagcggtttaggcttggaaaatattggcctggaagccgatagccgtggtttgctgaaagtgaacagcatgtaccaaaccgcgctgtctcatgtctatgctgtcggtgatgtgattggttacccaagtctggcatctgcggcttatgatcaggggcgtattgctgcacaagccatgatcaaaggtgaagccaacgttcacctgattgaagatatcccgacaggcatttacaccattcctgaaatcagttctgtcgggaaaaccgaacaagagctgacggcgatgaaagtgccctacgaagtgggccgggcgcaatttaaacatctggcgcgggcgcaaattgttgggatggataccggtagtttgaaaatcctgttccatcgggaaacgaagcaaattttgggtattcattgctttggtgaacgtgctgctgaaattatccacatcgggcaagctatcatggaacagaaaggagaaggcaatacgctcgagtatttcgttaatactaccttcaactacccaaccatggcagaagcctaccgtgtggccgcactcaatggcttaaatcgcctgttctaa

SEQ ID NO: 17 - DNA, 221 bp, Yersinia pseudotuberculosis
atgaaagcccatgtcagtagccgggagtatcaggataacggcaataaacgcatctataccctgatggatggcagcgtggtgatcgagtatcccaatctgccaggtaaatctcgtttcaatttctttaatcattgtgggaatacagttcacaaaaaccagcaacgtgtggccatgaaacaagccgttgaacaccataaaaaacagtggaaggtgaagccatg

SEQ ID NO: 18 - DNA, 36 bp, Artificial Sequence
  The nucleotide sequence was designed and synthesized
attagcatgcttgctggaaacgtttaaagcgtttgg

SEQ ID NO: 19 - DNA, 38 bp, Artificial Sequence
  The nucleotide sequence was designed and synthesized
attagaattctaattgtgcaaacaatctcacgcagttt

SEQ ID NO: 20 - DNA, 38 bp, Artificial Sequence; identical to SEQ ID NO: 19
  The nucleotide sequence was designed and synthesized

SEQ ID NO: 21 - DNA, 34 bp, Artificial Sequence
  The nucleotide sequence was designed and synthesized
attagagctcttgaaatcagcgccatccgccaat

SEQ ID NO: 22 - DNA, 34 bp, Artificial Sequence
  The nucleotide sequence was designed and synthesized
gatcgcatgcgaattctcatgtttgacagcttat

SEQ ID NO: 23 - DNA, 24 bp, Artificial Sequence
  The nucleotide sequence was designed and synthesized
gccgccgcaaggaatggtgcatgc

SEQ ID NO: 24 - DNA, 42 bp, Artificial Sequence
  The nucleotide sequence was designed and synthesized
attatctagaataattcactaaaaaatctgtttatcaatggt

SEQ ID NO: 25 - DNA, 34 bp, Artificial Sequence
  The nucleotide sequence was designed and synthesized
attagtcgacaagtgagtgagtgagtgagtgagt

SEQ ID NO: 26 - DNA, 3591 bp, Yersinia pseudotuberculosis
gtgagattgtttgcacaattaggttggtattttcgccgtgaatggcaccgttatgtaggggcggtgctactactgattattatcgctattctgcaattaattccgcccaagttggtgggcgttatagtggatggtatcagtacaaaacagatgtccaccaatatgttattggtttggattggcgtgatgctcgcgactgccgtggtggtctatttgttgcgttatgtctggcgagtcttattattcggtgcctcttatcagttggcggtggagctgagatctgatttttatcgtcagctgagtcggcaaactcccggtttttattcacgtcatcgcactggcgatttaatggctcgcgccaccaatgatgttgatcgcgttgtcttcgccgcaggtgaaggggtactaacgctggtcgattcactggtcatggggtgtgcggtattgattgttatgagcacccaaatcagctggcaattaacactgttatcgctattaccgatgcccatcatggcgatagtgattaagtattacggtgaccaacttcatcagcgttttaaatccgctcagggtgccttttctttgcttaataatcaggctcaagagagcctgaccagtattcgcatgattaaggcctttggtctggaagatcgtcagtcacaacagtttgctcaggtagcggtagaaaccggtgcgaagaatatgtatgtcgcccgcattgatgcccgctttgaccctacaatttatattgctattggtatcgctaatttattagctattggtggcggtagctggatggtggtgaataacagcattaccttagggcaattaaccagttttgttatgtatttggggctgatgatttggccaatgctggcattggcatggatgtttaatattgttgagcgcggtagtgccgcttatagccgcatccgcagtttgttggatgaagcacctgtggttaaagatggccacattactttgtctgatgtgcgtgacacattagcggttaatattcgtcatttttgttatccaggcagtgatcaaccggcactacataatgtggtactgacgctggttccaggggccatgctggggttatgtgggccaacaggttcgggtaagagtaccttgctggccttaatccagcggcaatttgatattgatgacggtgttatttgttatcaagggcatccgctgtcggatattcggttgaatgattggcgtggccgtttatcggtcgttagccagacccctttcctgttctccgattcggtggccggtaatattgccttgggtaaacccgatgcgacccccgcacagattgagcaggcggcccgcctggcctgtgtacatgaagatattttgcgtctgccgcagggttatgacactgaagtgggcgagcgtggagtgatgttgtccggtggtcagaaacagcgcatatctattgccagagcgcttttgttggatgctgaaatacttattcttgatgatgcactttctgcggttgatggtcagacggagcatgaaattttaaaaaacctgcgcgagtggggcgaacaacgtaccgtcattatcagcgcgcatcgcctttctgcattgaccgaagccagcgaaattctggtgatgcaacatggcggtgtaatgcagcgggggccccacagccttttagtcaatcagacgggttggtatcgggagatgtatcgctatcagcaattagaagccgcattggatgacggtgagcaggaggtcgaagccgatgaataaatgaataacgctcagcaactttggccgacattgaaacgcctgctttcctacggttcgccttatcgtaaaccgctggggctggcagtcttaatgctgtgggttgcggcagcagcagaggtaagtggtccactactgatcagttattttattgaccatgtagtcgccaaaggcacattacctctggggctagtcagtgggttggcattggcctatctgttgttgcaattattggccgcgacattgcactattttcaggcattgctgtttaaccgtgcggcagtgggggtcgttcagcggctacgcattgatgtgatggacgctgcattacgccaacctctcagtgcttttgatactcagcctgttgggcagttgatttcccgagtaaccaatgacactgaagtgatcaaagatttatatgtcatggtggtttctacggttttgaaaagtgcggccttaattagtgcgatgctggtggcgatgtttagtctggattggcggatggcgctgatttcaatttgtatcttccctgcggtgttggtggtgatgacaatctatcagcgctatagcacccctatcgttcgccgggtacgatcctatctggctgatattaacgatggttttaatgaagtcattaatggcatgggcgtcattcagcaattccgtcagcaggcccgtttcggggaacgcatggcgtctgccagccgtgcgcattatgttgcccggatgcaaaccttacggctggagggctttctattacgccctttattgagcctattctccgccttggtgttatgtggtttactgctgcttttcggtttcagccctgaaggttctgtgggggtgggtgtgctgtatgcgttcattaattaccttggccgcctgaatgagccgttgattgaactgacatcgcagcaatcaattatgcaacaggccgtggtggcaggagagcgtatttttgatctgatggatcgtgcgcagcaggactatggtagcgacaatattcccttgagcagtggccgtattcaggtggaaaacgtcagctttgcgtatcgctcggataaaatggtgttacacaatatttctctcagtgtcccctcccgtggatttgtggctttagttgggcacaccggcagtggtaaaagtacgctagctaacctgttgatgggctactatcccattcagcaaggtgagattttgcttgatggccgaccattatcccgcctctcccatcaggttctgcgccagggggtagcgttggtacagcaagatcccgtggtgttggctgactccttctttgcgaatatcacccttgggcgtgaccttagtgaacagcaagtgtgggaggcactcgaaaccgttcaattggcaccgttggttcgtaccttacctgatggcttgtacagtttacttggggaacagggcaataccttgtctgttgggcaaaaacagttgctggcgatggcgcgggtgttggtgcaagcgccccaaatcctgattctggatgaagccactgcgaatattgactctggcactgaacaggctattcagcgggcattacaggtgattcgaaaaaataccacgctggtggttattgctcatcgcctttcgacgattgtggaggcggatagtattttggtactgcaccgtggtgttgcggttgagcagggtaatcatcaagcgctgctagctgcccgtgggcgttattaccagatgtaccagttgcagttagtcagcgaagatttggctgccattgatcaggaagctattgataaaggctctattgatcaaagcactattgatcaagccggaatgagcgtgagttaa

SEQ ID NO: 27 - DNA, 2472 bp, Yersinia pseudotuberculosis
atgagcacttcatttttagtgggtgcccagcgttattcttttgatcccaatctgctagtggatggcaataacaacactgatacctcgttatttgaacagggcaatgaattaccgggcacctatttggtggatattatcttgaatgggaataaagtggactctacgaatgtgacatttcattcggagaaatcgccatcaggagagcctttcttgcaatcttgcttaaccaaggagcagctatcccgctatggtgtggatgttgatgcctatcccgagttatctccagcattaaaaaactcacagacaaacccgtgtgtcaatttagccgctatccctcaggccagtgaagagttccaattttataatatgcagttggtactgagtattccacaagcggctttacggcctgaaggtgaagtgccaatagaacgctgggatgatggtattacggcttttttgctgaactacatggcaaatatcagtgaaacccagtttcgtcaaaatggtggataccggcgttcacaatatatccagttatatcccggtttaaatttgggggcgtggcgtgttcgtaatgcgaccaactggagtcaatctggcgatagggggggtaaatggcaaagtgcttatacctatgccactcgtgggatttatcgtttaaagagtcgggtgaccttaggggagagttacaccccaggtgattttttcgacagcattccctttcgtggcgtgatgttgggggacgatcctaatatgcagccaagtaaccagcgggattttataccggtggtgcgtggtattgcccgtagtcaggcgcaggtggaaatcagacaaaatggttatctgatttatagtacggtggtgccgccggggccgtttgaactctctgacgtgatcccgagtaaatcaggcagtgacctacatgttagggtgctggaaagtaacggggcatcgcaggcgtttattgtgccttatgaggtgccagccattgcattgcgcaaagggcatttgcgatacaatctggtggcagggcagtatcgccccgccaatgctgatgttgagacgcctccggtcgctcaggcaaccgtcgcctacggcttgccgtggaacttgaccgcatttatcggtgagcagtggtcgcggcattatcaggcaacctctgccggattaggggtgctattgggggagtatggcgcattgtcatccagtatcacgcaggcaaccagtcagtatcaccatcaacaaccggttaaagggcaagcctgggaggttcgatacaacaaaaccttacaagcctctgatacgtcattttcattggtgaatagtcaatacagcactaatggtttcagtacgttatctgatgtattacaaagttaccggcagagcgggagtggcgataatagagataaaattgacgaaaattctcggtcaagggatttgcgtaaccaaatcagtgcggtcatcgggcagtcattgggcaagtttggctacctgaaccttaattggtcgcggcaggtctaccgtgggccgattccggctaagaattcattgggaatacactataacctcaatgtcggtaatagtttttgggcactgagttgggtgcaaaatgcgaatgagaacaagaatgatcgaatattgagtttgtctgtcagtattccgctgggaggtcatcacgatacttatgcttcctatagaatgacatcttccaacgggagcaatgatcatgagatagaaatgtacggccaggcttttgactctcgcttgagttggagtgtgcgtcaggcagagcattatggtcaacccaactcagggcataacagcggctcactcaggttgggctggcagggttcctatggcaatatcgcgggaaattactactatactccgagcatacgtcagttatccgctgatgtttccggaggggcgatcatccatcggcatgggctgacattagggccgcaaatcaatggcacctccgtgctggttgaagttccgggcgtcggcggggttaccaccacagaagatcgccgtctgaaaacagattttcgcggttacagcattgtgtctggcctctctccttatcaggagcacgatatcgtgttggaaactgcagatttaccgccggatgcggaggtggcaaaaacagataccaaggtcttgccgaccgagggggcgattgttcgtgccagttttagcccgcaaatcggggcaaaggcgctgatgacgatcacccgcgctaatggccaaaccattccgtttggtgcgatggcgagcttggttaatcagtcggccaatgcagccattgtcgatgaaggggggaaagcctatctaaccgggttaccggaaacagggcagttattggtgcaatggggtaaagatgcgggccaacaatgccgggtggattaccagctctcgccagcggaaaaaggtgatgccgggctgtatatgctcagcggagtatgccattaa

SEQ ID NO: 28 - DNA, 1425 bp, Yersinia pseudotuberculosis
atgtcactcagtcgtcgccagttcattcaggctgcgggcttagcgcttggtgctggctcactgccattgagggcacaggctagtagtactcagcaacctcagttaccggtgccccccttattagaatctcgccgcggccaaccgctttttttaaccttgcagcgcgcgcactgggcatttagtggcaataagaaagccgcagtgtggggaattaacggcatgtatctggggccgacggtcagagtttttaatggtgatgacgttaagctcatctacagcaaccggttgactgagccggtatcgatgacgatcagtgggttacaagtccctgggacgctgatgggcggagaggcccggatgatccaccctggagaggattggtcacccgtattgccggtacgtcagccggcggcaaactgctggtatcatgccaatacccccaaccgaatggctccccatgtctataacgggctagcggggatgtggctggtggaagatgcggtcagtaaagcgatgccgctgccaagtcactatggcgttgatgatttccctcttattattcaagataaacggctggataattttggtgtgccggaatataacccgcctgccaaaggggggtttgtcggggacacattattggttaacggggcgcaaagcccatttgttgaagtctctcgtgggtgggtacgtttacgtttattgaatgcgtctaacgcccgccgctacaccctgcaactcagtgatggccgtcctctgtatgttgttgccagcgatcagggttttctacctgcgccagtggcggtacagcaactttctcttgcaccaggtgaacggcgtgaagtggtgattgatatgtcacagggcaatgaagtctcgatcactgctggtgaatccgccggtattatggatcgcctgcgtgggttatttgagccatcaagtattttgatttctaccctggtgctgacattaaaaccaacggggttattgccattggtgacggataatttgcctatgcgtttattggcagatcagattattgaaggcagtgtgatccgttcccgtgaattccaactgggggataatctcccgggaattaacggcgctatctgggatatgaaccgagtagatgtgcaggctcagcaggggacatgggagcgttggataatacatgctgatatgccacaagcatttcatattcagggcgtttctttcctggtgaaaagtgtgaatggtgcggcggcaatggccgaagatcgtggttggaaggatacggtttgggtggatggtacggttgaattgtgggtgtatttcaatcaggtttcatcaccgcaattcccgttcttgttctacagtcagacgctggaaatggctgatcgtggctcggcggggcagttagtgacggtggcagcaccgacgctttga

SEQ ID NO: 29 - DNA, 2910 bp, Yersinia pseudotuberculosis
atgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatataa

SEQ ID NO: 30 - DNA, 477 bp, Yersinia pseudotuberculosis
atgaaaatgaaatgttttgcgaaaaatgcgctggcggttaccacactaatgatcgctgcttgtggtatggcaaacgcttctactgtcattaactccaaggatgtttctggtgaggtgactgtcaagcagggaaacacattccacgtcgattttgcgcctaacacaggagagatttttgcgggtaaacagccgggtgatgtcactatgtttacgctaactatgggtgatactgcaccacacggtggttggcgtttgattccaacaggggactcaaaaggtggatatatgatcagcgccgatggtgactatgttggtttatacagttatatgatgtcatgggtaggtatagataataactggtatataaatgatgactctcctaaagatataaaagatcatctgtacgttaaggcagggactgtccttaaaccaacgacttataaattcacggggcgtgttgaagagtatgtattttaa

SEQ ID NO: 31 - DNA, 822 bp, Yersinia pseudotuberculosis
atgaagaatctatttttttctgcatataaaaaagtattttcttatatcacatcaatagtcatattcatggtgtctttaccttatgcttattctcaagatgttgttgtcaatacgactaagcatctttttactgtaaaaatagggacgacgcgggtcatttacccttcgtcttcgacaaaaggggtatccgtatcggtggctaatccacaggattatccaatattggtacaaacccaagttaaagatgaagataagacgtcgcccgctccttttattgttacccctcctttattcaggcttgatgctgggttacaaggccgtgtacgtattattcggaccggtggaaaattccctgaagatcgtgaaacactgcaatggttgtgcctaacggggatcccacctaagaacggtgatgcttggggtaatacgcaaaataatccaaaaaattcatccccaactatggatattcaaatgtctatcagtacctgcataaaattattattcagacccgataaagttaaaggagatccgacggatagtgcggattcattaacttggagatataaaggtaactacttggaagttaataatccaacgccattctacatgaatttttattcactccgtattggtgatgaaaaaataaatttatctgatttaggttcaaaggatgaaataaaaaatggcagctatgttccgccattctcttctagggattttattattccggtaaaaaataaaggtaaggcaacagaggttttttggcaagtgattaatgataatggtggtgtgagccgggaatttaaaagcacagtccagtaa

SEQ ID NO: 32 - DNA, 822 bp, Yersinia pseudotuberculosis
atggatcagaagtttgcagtatttggtaatcctattagtcatagtaaatcgccgaggatccatacgcttttttccgagcaaacaggcattgaacatcgatatggtaaggtattagctccttctgaagcctttgagaacacattagtatctttttttgctgatggtgcccagggagcgaacattaccacaccgtttaaagaacgcgcatatgaccaatgtgatgaattaaccgaccgtgcatctctggcaggggctgttaatacgattaagcgtttggaagatgggcgcttgttgggtgacaacacagatggtattggtttgctcagtgatcttgaacgacaaaacttgatccgaacaactgatcatattttgttggttggggctggtggcgctgcccgaggagtgatccttcctctactttcttatgggtgtactgtggttgttaccaaccggacacatacccgggcgcagcaactggccaaagtatttaaccatattggtgatattgatgtttgtgaaatgtcggagttggctggacaacgattcgatttggttataaatgcgacggcatcaggccttcatggagaggtacctaacttaccagcagctatacttacttctcaaacacgttgttacgatatgttctatcaggcgggaaccacaccattcttggcttgggcacaacgattgggtttagcagattatgccgatggtttaggaatgctggtagggcaagcagcacatgctttcaaactttggcacggtgtgatgcctgaaattacgccagtattagcccagctacgcagtgagttaggtaaatag

SEQ ID NO: 33 - DNA, 3891 bp, Yersinia pseudotuberculosis
atggaaatactgcgtggttcacccgctttgtcggcttttcgtatcaccaaactgttgtcccgttgccaggatgctcacctgccggtgagtgatatctacgccgaatatgttcactttgctgatgtcagcgccccactgagtgctgatgaacacgccagactccagcggctgcttcaatatggcccatctcttcccgagcatccacccgcagggcgtttactgctggtcaccccaaggccgggtactatttctccatggtcttctaaagccaccgatatcgcacataactgtggattatcacaaattttacgtttagaacgtggtttggctttctctatccaaggcccgaatttgaacgagggtcagtggaaacaattagcggcgttattacatgaccgcatgatggaaactgtcttcaccgatttacagcaagcagagcagctattctctcatcaccaaccggcaccagtacaacgggtcgatattctggggcagggccgcagtgcattggagcaggccaatattaaattgggtctggcactggcgcaggatgagattgactacttactgaccgcttttaccggtttggggcgcaacccaacggatattgagctgtatatgtttgcgcaggcaaactcagagcattgccgccataaaatctttaatgcggattgggtcattgatggtgttgctcagccgaagaccctgttcaagatgatcaaaaacaccttcgaacacacccctgattatgtgctgtctgcatataaagacaacgctgcggtgatggaaggctctcaggtcgggcgtttctatgccacggcggagaaagggatttatgactaccatcaggaagaagcccatatcctgatgaaggttgaaactcataaccacccgacagcgatttcaccgtggcctggggcagcaaccggttcagggggtgaaatccgcgatgaaggtgcaaccgggcgtggtgccaagcctaaagcggggctggtagggttctctgtttcgaacctgcgcattccaggttttgagcagccgtgggaagagaacttcggtaagcctgatcgtatcgtgacggcgctagatatcatgactgaaggcccattaggcggtgccgcgtttaacaatgagtttggtcgcccggcgttgttgggttacttccgcacctatgaagagcgggttaacagccataacggtattgaattacggggttaccataagcctattatgttggcgggtgggttgggtaatattcgtgccgatcatgtgcaaaaaggtgaaatcaccgtgggggccaaattggtcgtactcggtggcccatcgatgaatatcggtttaggggggggcgcggcttcctctatggcatccgggcagtcggatgcggatctggattttgcatcggtacaacgggataacccagaaatggagcgccgttgtcaggaagttatcgatcgctgctggcagttgggcgagtataaccccattctgtttatccatgatgttggcgccggtggcctctctaatgccatgcctgaactggtcaacgatggtggccgtggtggccgctttgaattgcgggatatcctcaacgacgaaccgggtatgagcccgctggaagtgtggtgtaacgagtctcaggagcgttatgtactggcagtcgcacccgcacagatggccctgtttgacgaaatttgccgccgtgaacgcgcgccgtatgctgtcattggtgaagcgacagaagaaaaacatctgctcctgaatgatcgccatttcggcaatcaacccattgatatgccgctggatgtgctattaggcaaaacgccgaagatgctgcgtgatgttacccgcctacaggctaagggtgacgcgctgcaacgggctgatattagccttgccgaggcggtaaaacgcattatgcatttaccggcggtagcggaaaaaaccttcctgatcaccatcggcgaccgcacggtaaccggtatggtcacccgtgatcagatggttggcccttggcagatccccgtggctgattgcgcggtaaccagtgccagccttgatagctattatggcgaagccatgtcgctgggcgagcgcgctccggtggcattgctggactttgccgcctctgcccgcctggcggttggtgaagccttgacgaacatcgcagcaacccaaattggtgaattaaagcgtatcaagttatctgctaactggatgtctgctgctggtcacccaggggaagatgctggtttgtatgacgcggtacgtgcggtaggtgaagagctgtgcccagccctggaaatcacgattcctgtgggcaaagattcgatgtcgatgaaaacccgctggcaggaaggtcacgagcagcgcgaaatgacctcaccgctgtcactggtgatcaccgcttttgctcgcatagaagatgttcgccgcaccgtgacaccacaattgcgtacagataaaggtgataacgcgctgttgttgatcgatttgggcgcgggtcataatgcgctcggcgcgactgcgctgacgcaagtttaccgccagttaggtgataaaccagcggatgtccgtaacgtgcagcaactggctggcttcttcaacgccatgcagcgtttagtagccgatcaacatttgttggcgtaccacgaccgctccgatggcggcttgttggtcacgctggccgaaatggcctttgcagggcattgtggcgtaacggttgatattcagtctttgggtaacgatgctctggctgcactgttcaacgaggagctgggtgcagtgattcaagtccgtgctgagcagcgtgccgacgtggagaaattgctggctgaccacggtctggcaaattgtgttcactatctggggcgtgcggttgcgggcgatacctttgatattcgcagtggtactgacgtggtgtacagcgagaaacgcagcactctgcgcttgtggtgggcagaaacgagctggcaaatgcagcgtctgcgtgataatccggattgtgcggatcaagaacatcaggctaaacaggatgaaagtgatcccggcctgaatgtgaaactgacgttcgaccccgctgaagatatcgcggcaccattcatcctcaaacaggcgcggccaaaagttgctgtgctgcgggagcagggcgttaactctcacgttgagatggccgcggctttccaccgtgccgggtttgatgtggtcgacgttcatatgagtgacctgttggcgggccgcactgatttgcaatccttccaaaccttagttgcttgtggcgggttctcttacggtgacgtcttgggggccggtgaaggttgggcaaaatccattctgttcaatgatcgtgttcgtgatgaattcgaggctttcttccatcgcccaacgacattggcgcttggggtttgtaacggctgtcagatgatgtctaatctgcgtgaattaatcccaggtgcggagcattggccacggtttgtacgcaacttatcagatagctttgaagcacgtttcagcctggttgaagtggcaagcagcccatcactgtttatgcaggacatggtcggctcacgtatgccaattgctgtttcacatggtgaagggcaggtagaagtccgtgatgcggcacatttggctgctctggaacagagcaatctggtggcgttacgctttgtgaataaccacggggttgttaccgagcaatatccggctaacccaaatggttccgcgaatggtattacggcggtgaccagtgttagtgggcgggccacggtaatgatgccacaccctgagcgggtgttccgtacggtcagtaattcgtggcacccagaagagtggggcgaggatagcccatggatgcgtatgttccgtaatgcgcgtaagcaattaggttaa

SEQ ID NO: 34 - DNA, 1089 bp, Yersinia pseudotuberculosis
atggagaagattactgtcacgttaggggaacgtagctaccccattacgattgccgccgggttgtttaatgatccggcctcttttaagccgctaaaggcgggtgaccaggttatgctggtcactaaccaaacgttggctccgctctatctggattctctccgggcagtgttggaacacggtggcattaaagttgatcaggtgattttacctgatggtgagcagtataaatctctgagcgttatggagcaggttttttctgcccttctggaaaaaccgcacggtcgtgatactacgttggttgccttaggtggcggcgtagtgggcgacctgaccggttttgccgcagcttgctatcaacgcggtgtgcgctttattcaagttcctactactttactttctcaagtggattcttctgttggtggtaaaaccgccgttaaccatcccttgggtaaaaatatgattggtgccttctaccagcctgcatcggtggtggttgatcttaattgtcttaaaactctccccccgcgtgaactcgcttctggcttggctgaagtgatcaaatacggcatcatccttgatgcagctttcttcgattggttagaaaacaacattgacgctttattagcgctggatatgtcagcattagcttactgtattcgccgctgttgcgaattaaaggccgatgtcgttgctgctgatgaacgcgaagagagcggtgcgcgcgctttactcaatttgggtcacacctatggtcatgctatcgaagctgaaatgggctacggagtgtggttacacggtgaagcggttgctgctgggatggtgatggccgcacagacatcccgtcgtctggggcaactctctgtcagtgatgttgagcgtatcaagaaactcctattacgtgctggtctacccgtttgtgggcctaaagaaatggcaccagaatcttatctgccacacatgatgcgggataaaaaagtattggcgggtgaacttcgtctggtactgccaacggccatcggtaaatcagaaatccggggcggtgttgcgcatgatatggtgttggcatcgatagcggattgtcggccatag

SEQ ID NO: 35 - DNA, 1590 bp, Yersinia pseudotuberculosis
atgcaacaacgccgtccaatccgccgtgctctactcagtgtgtctgacaaagcaggtatcatcgaattcgcccaagcactttctcaacgcggtatcgagttactttccaccggtgggactgcccgcctgctggctgatgctggtttacccgttaccgaagtgtctgactacaccggcttcccggaaatgatggatggacgtgtgaagactttgcatccaaaggtgcatggtgggattttaggtcgtcgtggtcaggatgatggcattatggctcaacatggcattcagcccattgatattgtcgtcgttaatttatatcccttcgcccagacggttgcccgcccggattgctcgctggaagatgcggttgagaatattgatattggtggcccaaccatggttcgctctgcggccaagaaccataaagatgtcgccatcgtggtgaagagtagcgactaccccgccattattactgagcttgataataatgatggttcgttgacttaccccacccgtttcaatctggccattaaagctttcgaacacaccgccgcctacgacagcatgatcgccaactacttcggtacgctggtgccaccttatcatggtgatacggaacagccttccggccacttcccccgcaccctaaatcttaactatataaagaagcaggatatgcgttacggtgaaaacagccaccagcaagctgccttctatatagaagaagatgtcaaagaggcatccgttgccactgcccagcaattacaagggaaagccctctcttataacaatattgcggataccgatgccgcgctggaatgcgtgaaagagttcagtgaaccagcctgtgtgatcgttaaacatgccaacccatgcggtgtggctatcggtgattctattcttgccgcttatgaacgtgcctatcaaaccgatccaacctcagctttcggtggcatcatcgcctttaaccgtgaattggatgcagcaacggccaacgcgatcatcagccgccagtttgtcgaagtgatcattgcgccaacagtcagctctgatgcattggcattgcttgcagctaaacaaaatgtccgggtcctgacttgtggccagtggcaagcacgttcagcaggtttagatttcaaacgtgttaatgggggtttgctggtacaagaacgcgatttaggtatggtgacggcggccgaccttcgcgtggtttccaagcgtcagcctaccgaacaggaactgcgtgatgcgctgttctgctggaaagtggctaagtttgttaaatccaatgcgattgtctatgcccgcgataacatgacaatcggtataggtgccggccaaatgagccgtgtgtactctgcgaaaatagccggtatcaaggccgcagatgaagggctggaagtggctggctcagccatggcctctgatgccttcttcccgttccgtgatggtattgatgccgccgcggctgtgggcattacttgtgtcatccaaccaggcggctcaattcgtgatgatgaagtcatcgcggctgctgatgaacacggtattgccatgatcttcaccgacatgcgccatttccgtcattaa

SEQ ID NO: 36 - DNA, 1287 bp, Yersinia pseudotuberculosis
atgctggaatccctgaccttacaacccattgccctggttaatggcaccgttaatttacctggttcgaagagtgtctctaaccgcgcactgcttctggccgcgttggccgaagggaccactcagttgaataacgtgttagacagcgatgacatccgccacatgctcaatgcattacaggcattaggggtgaacttccgcctttctgctgatcgcacatgctgtgaggttgatggtctgggggggaaattagtggctgaacagccattgtcgcttttcttgggcaatgccggcaccgccatgcgtcctttggccgcggtgttatgtttgggtaatagcgatatcgtactgacgggtgagcctcggatgaaggagcggccaattggccatttggtggatgcgctacgtcagggcggtgcacagatcgattatctggaacaagaaaattacccgccattacgtttacgtggtggtttccgagggggggagttaactgttgatgggcgtgtctctagccagttcctgactgctttattgatgaccgccccgctggcggagcaagatacgactattcggattatgggtgatctggtttccaaaccttatatcgatattactctgcacttgatgaaagcatttggtattgacgtggggcatgagaactaccaaattttccacatcaaagggggccagacctaccgctcaccagggacttatttggttgagggcgatgcctcgtcggcttcctacttcttagcggctgcggctattaaggggggaacagtgcgtgtcactggtattggcaagaaaagtgtacagggcgacactaaatttgccgatgtgttggaaaaaatgggcgcgaaagtgacgtggggggatgattatatcgagtgcagtcgtggtgaattacagggcattgacatggatatgaaccacattcctgatgctgcaatgaccattgcgactacggcattatttgccacgggcccaacgacgatccgcaatatctacaactggcgggtaaaagaaactgaccggctgacggcgatggcaaccgagttgagaaaagtaggtgctgaagtggaagagggggaagattacatccgcgtggttccacccgtgcagctaactgctgcagatattggtacctacgatgaccaccgtatggcgatgtgtttctcgctggtcgcgttatcagacacccccgtgacgatccttgacccgaaatgtaccgcaaaaaccttccctgattattttgaacagtttgcgcgtctgagccaactggcctga

SEQ ID NO: 37 - DNA, 807 bp, Yersinia pseudotuberculosis
atggagcgttatcagcaactttttaagcagttagccgccaaaaaagaaggtgcctttgttcctttcgtccagcttggcgatccctctccggcgatgtcactcaatattattgatacgttaattgcggcaggtgcagatgcattagagttagggattccgttctctgatccgttggctgatggccctaccatacaaaatgcggcgttgcgtgcctttgctgcgggtgtcactccggctatttgttttgaaatactggccgaaatccgccaaaaacaccccacaatacctattggcctgttgatgtatgcaaatctggtattccacaatggtattgatcacttctaccagcgctgtgctgaggtgggtgtcgattcggtgctaattgctgatgtgccatttgaagagtctgcccctttccgggcagcggccttacgtcacgggattgcacccattttcatctgcccgcctaatgccgacggtgacttactgcgagaaatcgcttctcatggtcgtggttacacttacctgttatcacgtgccggagtcaccggtgcagaaaatcacggccagttaccgttaaaccatctggtagataaactgcgtgaatataatgcagcgcccgcattgcagggctttggtatttcagaacccgcgcaagtaaaagccagcctggcggcgggcgcggcgggtgctatttcaggctcagcaatcgtcaaaatcattgaaaaaaatgttgcacaaccagttgaaatgttagttcagctaacccgcttcgtcacagagatgaaagcggcaacccgcagctag

SEQ ID NO: 38 - DNA, 1068 bp, Yersinia pseudotuberculosis
atgagccagaaatttctttttattgaccgcgacggcaccatcattgccgagccaccaactgattatcaggttgaccggttggataaactggcgctggagcctgatgtcattcccgcattgctggcgttgcaaaaagcagactacaaactggtgatgatcactaatcaggatggcctcggcaccagcagtttcccgcaggaaaccttcgatccgccacataacctgatgatgcaaatcctgacgtctcaggggatcaattttgaacagatactgatttgcccacatctgccagccgataactgcacctgtcgcaaaccgaaaaccgcgctggtagaaagctatctggcagacggcgtgatgaacagtgccactagctatgtcatcggtgaccgtgaaactgacctacaactggccgagaacatgggtatcagcgggttacgttatcagcgtgatggcttgaactggacgcaaattgccaaacaactgacccagcgcgaccgccacgcctatgttaatcgcgtgaccaaagaaaccgccattgacgttaatgtttggctggatcgcgaagggggaagcaaaattaaaaccggcgtgggcttcttcgaccatatgctggatcaaatcgccacccacggcggttttcgcatggatattcaggtcagcggcgatctgtatatcgatgatcaccacacagtggaagataccgcgctggcactgggcgaagcgatcaacatcgcactgggtgacaaacggggtattggccgctttggttttgtattgccgatggatgagtgcctggcacgctgtgccttggatatttctggtcgcccgcatttggaatacaaagctgaatttaactaccagcgtgtcggcgatctaagcaccgagatggtcgagcacttcttccgctccctttcgtatgccatggcctgtaccttgcacctgaaaaccaaaggtcgcaacgatcatcaccgagtagaaagcctgtttaaagtatttggtcgtaccttgcgtcaagccattcgcgttgaaggcaataccctgccaagttcaaaaggagtgctgtaa

SEQ ID NO: 39 - DNA, 1287 bp, Yersinia pseudotuberculosis
atgaatattttgataattggtaacggcggtcgtgaacacgctctgggctggaaagccgcccaatctcctttagcggacaaaatttatgttgcaccaggtaatgcgggtacagcactggaaccgaccttagaaaatgttgatatcgccgccactgatattgccggtttactggcctttgctcaaagtcatgatatcggcctgacgattgttggcccagaagcccctttggtgatcggcgtggttgatgcgttccgcgctgctggtttagctatttttggcccgactcaggctgcggctcaattagagggttctaaagccttcaccaaagatttcctggcccgtcacaacattccctctgcggaataccaaaactttacagatgtcgaggccgcattggcctatgtgcgtcaaaaaggtgcgccaatcgttatcaaagccgatggtctggccgccggtaaaggcgtgattgttgcgatgacgctggaagaagccgaaaccgccgtaaatgacatgttggccggtaacgcttttggtgatgcagggcaccgtatcgtggtggaagagttccttgatggcgaagaagccagctttatcgtgatggttgatggcgaaaatgttttgccaatggcgaccagtcaggatcataagcgagttggcgatggtgataccgggccaaataccggcggaatgggtgcttattccccagcccccgtggtaacagatgatgttcaccaacgggtcatggatcaggttatttggccgaccgtgcgtggtatggcggcggaaggtaatatttacaccggtttcctctatgctggcctgatgatttcagccgatgggcaacccaaagtcattgagttcaactgccgctttggcgatccagaaacgcagccaatcatgttgcgtatgcgctccgatttggtcgaactgtgtttagccggtacacaaggcaaactaaatgaaaaaacctcagactgggatgagcgcccatcactgggggtcgttttagccgctggcggttatccagcagattaccgccagggtgatgttattcatggcttaccacagcaagaagtcaaggatggaaaagtcttccacgcggggaccaagctgaatgggaatcatgaagttgtcaccaatggtggccgcgtcttgtgtgtcactgcactcggtgaaaccgtcgcgcaggcgcaacaatatgcctatcagttagctgaggggatccagtgggaaggggttttctgccgtaaagatattggttatcgagcgattgctcgcggtaagtaa

SEQ ID NO: 40 - DNA, 714 bp, Yersinia pseudotuberculosis
atgcaaaagctagctgagttgtatcgtgggaaggcgaaaaccgtctataccaccgaaaatcctgacctactggtattggagttccgtaacgatacgtcagcactggatggtcagcgcattgagcagttcgatcgtaaaggtatggttaataataagtttaaccatttcattatgacgaaattggaagaggcgggtattcctacccaaatggaacgcctgctgtcagataccgaagtactggtgaaaaaactggagatgatcccggttgagtgcgttattcgtaaccgtgctgcaggttctttagtcaagcgtctggggatcgaagaaggtttgtcactgaatccaccgttgtttgacctctttttgaaaaatgacgcaatgcatgacccgatggttaatgagtcctactgtaaaacctttggttgggcgacagaagcgcaattggcccgaatgaaagaactgagctatttggcgaatgatgtgctgagcaaattgtttgatgatgccggtttaattctggtggatttcaaacttgagttcggtctgtttaacggtgaagtggtattaggggatgaattctctcctgatggtagccgtctgtgggataagaaaaccctgaacaagatggataaagaccgttatcgccagagcctgggcggtttgattgaagcctacgaagaagttgcgcaccgtatcggcgtaaaattagactaa

SEQ ID NO: 41 - DNA, 1389 bp, Yersinia pseudotuberculosis
atgctattacctgtaattatggctggaggtgctggtagccgtttgtggccattatcccgagctctttatcctaaacaatttctagcgctaacgtcagatttgacgatgctacaagaaaccctattgcgtctggacggccttccccaccttgcaccattagtgatttgtaacgaagaacatcgctttattatcgcagaacagttacgtcagaaaaatctggtgcatagcggaatagtcttggaacctgttgggcgcaataccgcgccagctatagcattggctgccttacgagcaacaatgagtggggatgatcctctactattggtattagcagccgatcacgtgattcaggataaacttgcatttattcgtgccgtccaacgtgctgaaccgcttgctgaagcgggaaaattggttacttttggaatcgtgccaaagagtccggaaacaggatatggatatattcgccaagggaagcaagtcgtagatggcgcttatcaggttgctgcttttgttgagaagccagatctgattactgcagagcggtatttggcttcgggtgactattattggaatagcggtatgtttgcatttaaagcatctcgctatctacaggaattagctctacatcgcccggatattttggctgcctgcaagcaagccattgctggtcaacatactgatttagattttattcgtctcaatgaagaagctttctctagttgccctagtgaatctatcgactatgctgtgatggaaaaaactagcgatgccgttgtagtgccactggatgcacagtggaatgatgttgggtgctggtcagcgctttgggaaattaatactaaagatgaccatggaaatgttattcgtggtgatgtattaatggaagatactaataatagctacgtttattctcaaaataggctcattgcaactgtaggcattaatgatttggttattgttgaaactaaagatgccattttggttgctcataaagataaagtacaaaatgttaaagggatagttggacaacttaagcttgaatctcgatgtgaatatctacagcaccgggaagtctatcgcccttggggttcgcatgatgctattgctgaaggtattcgctaccatgtgcagcatgtaacagtgaaaccgggtcagcgcattacgactcaaattcattatcacagagcagaacattggattgtagtttctggtacggccttggtaactattgcagataagacaattatattatgtgagaatgagtcaacatttatccctattggcaaacctcactctttagagaatcccggttctattccgcttgaaattatagaggttcagtcgggttcttatttaggtgaagatgatattatccgtttagaataa

SEQ ID NO: 42 - DNA, 1233 bp, Yersinia pseudotuberculosis
atgacaactttatcaccggcaattccggtcaatcgccgtagtgaaatgctgtgcagcatagcagcgttactcttaggtattagtatgccaacgaataatcagctaatgagtgtttcattggttttgattattatcagtttgatcattaaccgaaaatcccttgattttaaaccgttgctcaccagtccattagtctatctaccggcagctatgtttgtgttactggcgttatcattgttgtatcaaaacaacagctatggcccggatatggtaggaaaatataagaaacttctttatatcttgccattggccctgttctttatgaaccagccacggttgatcaaactgttctgtaccggctttctcgtcgctaacgctgtcatattggccggatctctggccgtgggtgttttacacattccgttgagtggtgtggaccccactaacccgaccatttttaaactgcaaattacccaaaacttctttatggcgctggcggcgttactctggctggtactggcgtttcaacatcaggggtggaaacgttggggttacagtgtgttagtggtggccgcaagttacagcatcttattcttggtattgggccgcaccggttatgttgccttgatagtgggtttaggggtatggctatttttctctctgggcaatcgccagcggttaacactggtcgtgctaggtgcattggcctttgctgccctgatctttatcccgaacaaagccacagatcgtatcgtacagggcgttgatgagataaaagtgtgtatggccgcatcggcaactgatgccgcagacgcatgtaattcctccatggggcagcgttctgcttttgtggttgaagctgcccggttaattaaagaatcacctattcttggtcatggcgcgggtggtttttattatgaaaataaagaagtggactacaaagtcaataacccgcacaaccagtatctactggaaacgattcaatcaggtgtgattggtctattcctctttttggcgtgggttatctgctgttatcgtgttatttggcagcaaacaccggctctgcgcaatgtgctgttggcggtattgaccagttatatggcatgtaacttcttcaattcatttctgctggattcgtctgaaggccacctgtttatgatttttgtggcggttctggcgggctattcggtgagcggttctcaatccatagcaggtaaacggctgccaacgtag

SEQ ID NO: 43 - DNA, 1083 bp, Yersinia pseudotuberculosis
atgagtactgaattaatttatatctttctgttttctatggcatttctatttgttgctcgcaaagttgccataaaaattggacttgtcgataaacccaactaccgtaagcgccatcaaggattaatccctctggtggggggaatatcggtattcgcgggggtctgttttgcttttctgattactaatcagcaaattccccattttcgtttatatcttgcatgtgccgggttattggtcttcgttggtgctttagatgatcgttttgatatcagcgtaaaaattcgcgcttttgttcaagcactggtcggcatcgcgatgatggcagtcgcggggctatatttacgtagtctggggcatgccttcggtccatgggaaatggtattagggccatttggttatgtggtcaccctgtttgcggtttgggcggccatcaatgcgttcaacatggttgatggaatagatggcttgctgggtggcctatcctgtgtttccttcggggccatggggattttgttgtaccaaagtgggcagatgtcattggcgttatggtgtttcgccatgatagccaccattattccctatatcttactgaatctcgggttactgggtcgccgctataaagtcttcatgggcgatgctggcagtactttgattggtttcaccgcgatctggatcctgttacaggcgactcagggtaatgctcatccgatcaaccctgtgacggcactatggattatcgccattccgttgatggacatgattgccatcatgtatcgtcgtttacgcaaaggcatgagtcctttctctccagatcgccaacatattcatcatttaatcatgcgggccggttttacctcccggcaagcttttgtcctcatcacactggctgccgcgttgttagccatgattggtgttatcggcgaacggctgacatttatacccgaatgggtcatgttggcattgttcttgcttgcatttctattgtatggctactgcattaaacgggcatggcgggtggcgcgttttatcaaacgtttcaagcgtcggatgcggcgggcatcgaaaaataagcatgaatcttaa

SEQ ID NO: 44 - DNA, 486 bp, Yersinia pseudotuberculosis
atgaaatcttggtatttattgtattgtaaacgcggtcagattttacgtgccaaagagcatttggaacggcaaactgtaaactgttggacaccaatagtggctatcgaaaagatagttcgagggaaacgtattgaggtaatagaggctctattccccaattatttattcgctgagtttgacccggaaaatatccataccacaaccgtcagcgccacccggggggtcagccattttgttcgttttggtacacagcccgcggtgatcccagcaaccgtcattgctgatatgcaagcccatgctgtggataagataattgcccctgaagtaccgaaacctggtgatatcgtaaaaattattgatggcgtttttgccggattacaggctatttataccgaaccagatggtgaagcccgctcgatgttgttactgaatatgctcaatagccaaattaagcatagcctggacaatcgtcagttcgaaaagtaa

SEQ ID NO: 45 - DNA, 1014 bp, Yersinia pseudotuberculosis
atgaagataatttacgatgggattattaattccttacaacgtactggtggtataactatttattttaaagagttagtgactcgtcttccagaaaggtattttgactggtattcatacgatgttaagttgggtgatattggtgttgatggtattgaactgaaatctaggttattagaacgttatagagatttttcaataaaaaatgttagtgataagtctcctgatatatttcattcatcatattatcgtctgccaaagtttgatattccgatagtcacgacagttcatgattttacatatgaaaagttcattaatggccctgcgaaatgggtgcactcctggcagaaaaaccgtgcggttaacaatagtgatttaattatttgtgtctcggaaaatacggctaaagacttacagaagtattgttctgtttcgagtgaaaagattagaattgtacataatggcgtgtcagaaaaatatcattccattactactgttacaagttacacgaataaagttatttttgttggtgcgcgtggtggttataagaattttgacattgcagtgaaagcaatatcaaaaacacctcacctcgaattatcagttgtaggtggaggggcattcactagtaaagaactgtcactactgaatcactatttacctgggcgctaccatggattaggacgtctgagtgacgaggccttgaatgaggcatataattcagcttatgcgctgctttacccatctagctatgaaggttttggtattccaatattagaagcgatgagtgcaggatgtcccgtaatatctgttaatgtatcttctatacctgaggtcgcaggtgatgccgctatattagtgcaaaaaccgactgttgatgaactagttgacggcttgcttgccgtagaaagtgaaaggtctaaacttattggctatggcatgaagcaagcggctaagttctcatgggataagtgttatcaagaaaccttagatgtttataaagaattgaacaaataa

SEQ ID NO: 46 - DNA, 1074 bp, Yersinia pseudotuberculosis
atgattaataatagtttctggcaaggtaaacgggtttttgtaacaggccatactgggtttaaaggtggctggttgagtttatggttgcaaaccatgggggcaacggtaaaaggttactctctgaccgcccccactgtgcctagcctatttgagaccgcacgagttgccgacgggatgcaatcggaaatcggtgatattcgtgatcaaaacaaattattagaatcaatccgcgaattccaaccagagattgttttccacatggctgctcagccactggtccgtctatcctattccgagcctgttgaaacctactcgacgaatgttatgggtaccgtttatttactggaagctattcgccatgttggtggcgtcaaagcggtggtcaatatcaccagtgataaatgctacgataataaagagtggatctggggctatcgcgaaaatgaagcgatgggggggtatgatccttactccaacagtaaaggttgtgcggaattagtgacgtcatcctaccgtaattcgttcttcaatccagcgaactatggccagcatggcactgccgtagcgacagtgcgtgcgggtaatgtcatcggtggtggcgattgggcattggatcgcatcgttccagatattcttcgggcgtttgaacagtcccaaccagtgattattcgcaacccacatgccattcgcccatggcagcatgtgttggagcctttgtcgggttatttgctgttggcacagaagttatatactgacggtgctgaatatgccgaaggttggaactttggtcctaacgatgctgatgctactccagtaaaaaacattgttgaacaaatggtgaaatattggggagagggtgcaagctggcaattagatggcaatgctcaccctcatgaagctcattatctgaaactggattgttcaaaagctaaaatgcaacttggctggcatcctcgctggaacttgaatactacgctcgaatatattgtgggctggcacaagaactggttatcaggcacagatatgcatgaatacagtattactgaaattaataattacatgaacactaaatga

SEQ ID NO: 47 - DNA, 960 bp, Yersinia pseudotuberculosis
atgaaaatagctttgattggtggttctggttttattgggacaaatttggcccgattattaatagataatagtgttgatttttctattttagataaagttaaaagtgatgtatatcctgagcgatgggtatattgtgatgtcacagattatgatagtttaatatctactcttatcggtcatgatttaattataaatttggctgcagaacataaagataatgttaatcctattagtctctactatcaagttaatgttgagggagctaaaaatatttgtagggctgcggatagccttaatataaagaatattgtatttacatcttctgtagctgtttacggttttgttgaaaaagatacagatgaaagtggaaaatacgctcctttcaatcattatggaaaatctaagttagaagcagaaaaggtctatgattcttggtttaattcttctgcagacaaaaaactagttactcttcgtcccacggttgtttttggtattggcaacagaggcaatgtatataatttatttaagcaaattgcatcaggcaagtttgttatgattggtcgtggtgagaacgagaaatctatggcctacgtggagaacatagctgctttcttagttcttactctatcatttcctgctggatatcatttaataaactatgtggataagccagacttcactatgaatgagctagctaatgtcatttatacatgtctcgggaaaaagagtaagatcgttagagtcccttatttttttggtctctttgcaggttatatatttgatctgttggcgaaaattactggcaaagagttacctgtaagtagtattcgaattaaaaaattctgcgctaaaacacagtttagttcaaaaaatattgagaattataaattttcggctccttactctttacaagacgctgtagtgaaaacaatctcacaagagtttatgagtgagaagtaa

SEQ ID NO: 48 - DNA, 1122 bp, Yersinia pseudotuberculosis
atgactaagattgcgctcattactggtattacaggacaagatggctcttatttggctgagtttttattagaaaagggttatgaggttcatggtattaagcgccgagcttcatcttttaataccagccgcattgatcatatttatcaagatcgccatgagacgaatccacgcttctttttacattatggtgacttgactgacacgtcgaatttaattcgtttggtccaggaaattcagcctgatgagatttataatttgggtgcacaatctcatgtggctgtttcatttgaatcaccggaatatactgccgatgttgatgccatggggacactgcggttgctagaggccattcgtatcaatgggcttgaaaagaagactcgtttctatcaagcctcaacctcagagctttatggtttagtgcaggaaacgcctcagcgtgaaacaacgccattctatccgcgttctccttatgctgttgccaaaatgtatgcttactggattacagtaaattaccgtgaatcatatggaatgtatgcttgtaacggaattttatttaaccatgagtctcctcgtcgtggtgaaacatttgttactcggaagattacccgtgctgttgcgaatattgcattaggtctagaaaaatgtctctatttaggcaatatagattcgctacgtgactggggccatgcgaaagattatgtgcgtatgcaatggatgatgttgcaacaagataagccagaagattttgttattgcaacaggcaagcaaattaccgttcgtgagtttgtacgtatgtcagcaagagaagcagggattgaattggaatttagtggagaaggcgttgaagaggttgccactgttgttgctattaatggtaaccatatttcttctgtcaatattggagatgtaatagtccgtgttgatccacgttatttccgtcctgctgaagttgaaacattattaggcgatccaactaaggctaaaaaagtattaggctgggtacctgaaattacggttgaagaaatgtgtgctgaaatggtcgctagtgacttagagcaggctaaacagcatgcattgttaaaggctaatggctttgacgtatccatttcgttggagaattaa

SEQ ID NO: 49 - DNA, 966 bp, Yersinia pseudotuberculosis
atggataagaaacgcgtatttattgccgggcatcgtggcatggtgggttctgctattgtacgtcaacttgaaaaccgtaatgatattgaattgatcattagggatcgtactgaacttgacctcatgtctcaatccgctgtgcaaaaattttttgctactgaaaaaattgatgaaatctatttggctgcggcaaaagtgggggggattcaggccaataataattatccggcagagttcatctaccaaaacttaatgatcgagtgcaatattattcacgcggctcatttagctggcattcaaaaattattatttttggggtcttcttgtatttatccaaaattggctgcacaaccaatgacagaggaggctctgttaactggcgtcttggaaccaacgaatgaaccttatgccatcgccaagatagccggtatcaaactgtgcgaatcttataatcgtcaatatggtcgcgattatcgcagtgttatgccaaccaacctttatggtgaaaatgacaattttcaccccgaaaattcccatgtcattcctgccttattacgtcgcttccatgaggctaaaattcgtaatgataaggaaatggttgtgtggggaacgggtaaaccaatgcgtgagttcctgcatgtagatgatatggctgctgccagtgtgcatgtcatggagctgtctgatcaaatttatcaaaccaatactcaaccaatgctttcgcatattaatgtcggaacgggtgtggattgcactattcgtgaattggcagaaactatggctaaagttgttggtttcaccggaaatttagtttttgattcaactaagccggacggaacaccacgaaaattgatggacgtaagccgcttggctaaactcgggtggtgttatcagatttcgcttgaagtaggtttaacgatgacttatcaatggttcttggctcatcagaataacttcagaaaatag

SEQ ID NO: 50 - DNA, 1314 bp, Yersinia pseudotuberculosis
atgagtcaagaagaattacgtcaacagattgctgagctggttgctcagtacgctgaaacggctatggcccctaagccatttgaagcaggtaagagtgtcgttccaccttcaggtaaagttattggtaccaaagaactccagttaatggttgaagcttctttagacggttggctaacaacgggccgttttaatgacgcttttgagaaaaaactaggcgagtatttgggcgttccttatgttctgactacaacttctggctcttcagccaacttattggctttgaccgcgctgacctcacctaaattaggggtacgggcgttgaagccaggtgacgaagttattactgttgccgcaggttttccaaccacagtaaacccaactattcagaatgggttaattcctgtctttgttgatgttgatattccaacttacaatgtaaatgctagcctgattgaagcggcggttagtgataaaaccaaagctattatgattgcccatacattaggtaatctattcgatctagctgaagttcgccgagtagctgataaatataacctgtggttaattgaagactgctgcgatgcgttgggttccacctacgatggaaaaatggctggtacatttggcgatattggtaccgttagcttctatcccgctcatcatatcaccatgggtgaaggtggggcggtatttacacaatcggcggaactgaagagtatcatcgaatctttccgtgattggggtcgtgattgttattgtgctccaggctgtgacaacacatgtaaaaagcgtttcggccagcaacttggctctttaccattcggttatgatcataaatatacttattcccatttaggctataacctaaaaatcacagatatgcaggctgcctgtggtttggcgcaactagagcgcatagaagagtttgttgaaaaacgtaaagctaactttaaataccttaaagacgcactccaatcttgcgctgactttcttgagttaccagaagcgactgaaaattcagatccatcatggtttggtttccctatcactctgaaagaagatagcggagttagccgcattgatctggttaaattccttgatgaagctaaagtgggaactcgcctactatttgccggtaatttaactcgccagccgtatttccatgatgtgaaataccgtgttgttggtgaattgacaaacaccgatagaattatgaatcaaactttctggattggtatttacccaggcctgacacatgatcatttggattatgttgtgagtaagcttgaagagttctttggtttgaatttttaa

SEQ ID NO: 51 - DNA, 1263 bp, Yersinia pseudotuberculosis
atgagttttgaaactatttctgttatcggtcttgggtatattggtctgccgaccgcggcggcttttgcttcgcgtaaaaagaaagtgattggcgttgacgtcaatgcgcatgcggttgaaaccattaatcgtggtgccatccatatcgtggagccagatttagacaaagtggtaaagattgctgttgaaggcggctacctgcaagcggtgacgaaaccacaggcagcggatgcattccttattgccgtaccaacaccgtttaaaggcgatcatgagccggatatgatttttgtcgaatcagccgctaaatccatcgcaccggtgctgaaaaaaggcgatttggtcattttggaatctacctccccggtaggtgctactgagcaaatggcgcaatggctggctgaggctcgccctgatttgagtttcccacagcaagcgggcgaagcagccgatatcaatattgcatactgccctgagcgcgtgttaccgggccaggttatggttgaactgatccagaatgaccgggtgattggtggcatgacaccaaaatgttctgcccgcgccagtgcgttatataaaattttcttggaaggtgagtgtgtggtgactaactcacgcaccgctgagatgtgcaaactgacggaaaacagcttccgtgacgttaacattgcctttgccaacgagttatcactgatttgtgatgagcaaggtatcaatgtgtgggagctgattcgtctggcgaaccgccatccacgcgtgaatatcttacagccgggtccaggtgtaggtggtcactgtatcgccgtcgatccatggtttattgtttctcagaatccacaattggcccgcctgatccacacggcgcgtctggtaaacgacggcaaaccgttgtgggtggttgatcgcgttaaagccgctgttgctgattgtctggccgccagtgataaacgcgcttctgaagtgaaaattgcctgcttcggtctggcttttaaacccgatattgatgacctgcgtgaaagcccagcagtgggtgttgcccgcttgatcgcggagtggcatgtgggtgaaacactggttgttgaacccaatgttgagcaattgccaaaatcactgatgggtcttgtcaccttgaaagataccgccactgctttacaacaggctgatgtcttggtcatgttggtggaccacaaacaattcaaggccatcaaacctgaagatattaagcaacagtggattgttgataccaaaggagtatggcgttga

SEQ ID NO: 52 - DNA, 906 bp, Yersinia pseudotuberculosis
atgaagcaagttggcctgaggattgatgtcgatacctaccgaggaacgcagtacggggtgccatcattactgactgttttagaaaaacatgacattcgtgccagttttttcttcagtgttgggccggataatatgggacgccatttatggcgcctttttcgtccgcgctttttatggaagatgctacgttctaatgctgcctcgctttacggttgggatattttactggcgggcacggcttggccaggtaagaagatcgctaaagactttggtccgttaatgaaagcagcggccatggcggggcatgaagtggggttgcatgcttgggaccatcaaggctggcaggccaacgtcgcttcatggtcacagcaacaactgactgagcaagtacagagaggagttgatactttacagcagagtattggtcaacccattagctgttctgctgctgcggggtggcgcgccgatgaacgggttcttgcggtaaaacagcaattcgattttagttataacagcgattgccgggggacccatcctttccggcctttgttgcctaatggcagcttagggagtgtacaaattcccgtgacgctgccaacttacgatgaagttgtcggcggtgaggtgcaagctgaaaacttcaatgactttattattgacgccatcctacgtgatagcggggtatcggtatacaccattcatgctgaagttgaggggatgtctcaggcagcaatgtttgagcagctcttgatgagagcaaagcaacaagatattgagttttgtccgctcagtaagctgctaccgtcagatttacaattactgcctgtgggcaaagtaatacgcgctacatttcctggccgggaaggttggttgggttgtcaatcagacataaaggatgctgaatga

SEQ ID NO: 53 - DNA, 750 bp, Yersinia pseudotuberculosis
atgaaaatatccataattactgctacatataatagtgcgtcaactattgtagaaacattagattctttgaatgaacaaacatatgataatatagaacatattatcattgatggtggctccacagataacacgcttgaacttgtaaaagcgtatgggaaacgtgtttcaattgtgatatcagaaaaagatgaaggaatttatgatgcattaaataaaggtatttccgttgctactggtgatattattggtattttacattcagatgatatgtttgcttatatcgatgcagtaagtgatattgcaaaagtatttttcgataatactgtggatgcttgctatggtgatttatcatatatatcgagatctggagataatcgagtcgtaagaacttggatcgcaggtgactactcagaaagtaaatataaatatggttggatgccgccacatacaaccttttatatgaaaaatgcactatacaaaaaactaggtggatatgatactacacttaaaatagcagcagattatgatgccatgctacgttatactttggtagcaaaaatcaatattgtgtatatacctaagattttaatttatatgaaggttggaggggtgagtacacggttatcacagaaatttgagtcattaaaagatgagttaattgtaatgaaacgctataatttaggtggagttatgacctttatgaaaaagaaaatttacaaactccctcagttttttaaaattgtcaaatag

SEQ ID NO: 54 - DNA, 858 bp, Yersinia pseudotuberculosis
atgaaaattcttattaccggcgttagcggttatttggggagccaattagccaatgctttaatgctagagcatgaagttgcgggaactgtgcgtgctggttctgtatgtaatcgcatcactgatattggtaatgttaatttaattaatgttactgacagtggctggatagataaggttttgtcattttctcctgatgtggttattaatactgtagcattatatggaaggaaaggagagttactttcagagctggttgatgccaatattcaatttccattacgtatattagaaatgttagtttcaacaggaaaaggcttgttttttcagtgcggaacatctctacctgcggatgttagtcaatacgctttaactaaaaatcaattcactgagcttgccagagaatattgcaataaatttagtggtaaatttatagagttaaaattagaacatttttttggtccttttgatgattcaactaaatttacaacttatgtaatcaatagttgtagaagtcatagtgacctaaaattgacagccggcttacaacgccgtgattttatttatattaatgatcttatcaacgctttcaaaattatgatttcaaaatcagagagtctaatttcaggcgaaagtatttccattgggtccggtcatgctgtcacgataaaagaatttgtagaaactgtcgcaaagatgacaagttaccaaggcaatctacagtttggtgcaatccctactagagagaatgaattgatgtatagctgtgcatcattggcgagaatacaggaactcggttggttgtgccaatattcattaaatagtgcaattaaagatacactaaacagaatgcgggtctga

SEQ ID NO: 55 - DNA, 1152 bp, Yersinia pseudotuberculosis
atgagtaataaacaggcacgaagtaaccttgatagtccaataccgaataactatgatttttcaaatgtatcttcttcaagaaatgaaattgatctctttgaaatttttggtgttgtatttaaatcaaagttcaagataatattaataacactatttttcttaattagtggtttagtggtctcatatatcctcccccaaaaatggacaagcactgcaataatagctcttcctggtgatgagcaagttcaagttctggatgaactaatcacaaatctgaccgtgcttgatataaaggttgatgtgagtgctaattatttgctgtcaacattcaaacaaaatttcgattctcaagatctccgtgaacaatatttagtaaatactaattactttaaacgtttgatgaagaataatccagaagatggtttggataaaagagcgttaatagagcgaatcgtaaatgaaaatatttcttcggttaacccattgaaagataaaaccgagggtgaaaatgaatatcgctattataaattatcatatagtgcaagcacaccgacagacgctcgtgacttgttgcaaggctctattaactatgtaaataccatcgttaatgctgatgttttccgaaaaatacagcgagcagtggatttagccaagggtatcggtacagataaatactctatggaattgttgaaagctagaaataaccaaaaagttaaaattgagcgcttaaggtatgcttcttctatcgctgatgccgcaggcgtaaaaaaaccagtttacagcaatggctcagccattagtgatgatccagacttccctattactatgggatccgatgcgctgaaccgtaaactggaaatagagaagtcagttatcgatttggcttcaatcaatactgaacttctaaaccgtaagttgtatttggataaattaaataggttagaaattcctaatgttaatatcgtgccatttaaatatttgcaacagccaacggaacccactaaaagagatgtccctaagcgcgcattgattgtgattctgtttgccctggtcggtcttatgggttctgtcggttttgttttagttgagcactttgtgcgtgaacggaagcgagaagaagaggggcttaagctctctcaaactaaggaatag

SEQ ID NO: 56 - DNA, 1665 bp, Yersinia pseudotuberculosis
atgaaattattaaaagacagtggcgcagcattattggcgctattttttgtattggtttatttgttaccggtgaatagccgtttgttatggcaacccgatgaaactcgctatgctgaaattagtcgtgaaatgctgcagcgtggcgactgggtcgtgccgtatttcatggatatacgttattttgaaaaaccggtcgccgggtattggtttaataacattagccaatggatctttggtgacagtaattttgcagtgagatttggctctatttttagcacggcattgagtgccgtattagtttattggctggcaaccttattatggcgtaatcggtcaacctcggtactggctacattaatttatctttcctttttattggtgtttggcattggtacctatgcagtgctggaccccatgatttctctgtggttgacagcggcgatggtgagtttttatctcacgctaaaagccgaaaattggcaacagaaagtcggcgcatatgcattacttggggttgcttgcggtatggggtttatgaccaaaggctttctggcattagcggtcccggtgattgcggtattacccattgtcatccagcaaaaaaggataaaagaccttgttgtttttggtccgatagcaattgtgtgcgcggtattattaagtttaccgtgggctttggcgatcgcccaacgagaacctgatttctggaactatttcttttgggttgaacatattcagcgctttgcggaagcgagtgctcaacataaatctcccatttggtattacctacccattttatgtattggtgtcctgccttggctagggctattacctggagccttatttaaaggttggcgtgagcgggcaacaaaaccagaattattctttttactcagttgggtggttatgccactcctattttttagcgtagcaaaagggaagctaccgacttatattttgccctgtatggcacctctatccttgctaatggcggcatatgcgacggattgcgctaataatatacgtatgcgggcattgaaaataaacggtgtaataaatctactcttcggggttgcctgtgcgttagtcatcgtcgttattggcttggggttagtgaaggatatcgtggcttatgggccgcaagagaatcagaaagtttggttaggtgtgttggcatttgctggttggggagtcactgggtttattaccttacgcaataacgcccgaaactggcgctgggcagcagcttgcccactgttattcattttgttagtgggttacctaattccacaacaagtggtggactcaaagcaaccgcaaaactttatcaaaaataattttagtgaactcagctccagccgctatgtgctgaccgacagcgttggtgttgccgctgggctggcatgggagctaaaacgcagtgatatactgatgttcagtgaaaaaggtgagctaacctatggtttggcctacccagacagtcaggataattatatcagtaatgacgatttccccacatggcttgcgcaggcgagaaaaaaaggcgacgtctcattggtcgtgcaattagccagaaatgaagcactcccggcccatctgccgtcagcggataaggtgaatttaatgaatcgactcgccctgttgtggtaccagaaaacaccatga

SEQ ID NO: 57 - protein, 8 aa, Artificial Sequence
  The amino acid sequence was design and synthesized
DYKDDDDK

SEQ ID NO: 58 - protein, 607 aa, Yersinia pseudotuberculosis
MNNAQQLWPTLKRLLSYGSPYRKPLGLAVLMLWVAAAAEVSGPLLISYFIDHVVAKGTLPLGLVSGLALAYLLLQLLAATLHYFQALLFNRAAVGVVQRLRIDVMDAALRQPLSAFDTQPVGQLISRVTNDTEVIKDLYVMVVSTVLKSAALISAMLVAMFSLDWRMALISICIFPAVLVVMTIYQRYSTPIVRRVRSYLADINDGFNEVINGMGVIQQFRQQARFGERMASASRAHYVARMQTLRLEGFLLRPLLSLFSALVLCGLLLLFGFSPEGSVGVGVLYAFINYLGRLNEPLIELTSQQSIMQQAVVAGERIFDLMDRAQQDYGSDNIPLSSGRIQVENVSFAYRSDKMVLHNISLSVPSRGFVALVGHTGSGKSTLANLLMGYYPIQQGEILLDGRPLSRLSHQVLRQGVALVQQDPVVLADSFFANITLGRDLSEQQVWEALETVQLAPLVRTLPDGLYSLLGEQGNTLSVGQKQLLAMARVLVQAPQILILDEATANIDSGTEQAIQRALQVIRKNTTLVVIAHRLSTIVEADSILVLHRGVAVEQGNHQALLAARGRYYQMYQLQLVSEDLAAIDQEAIDKGSIDQSTIDQAGMSVS

SEQ ID NO: 59 - protein, 588 aa, Yersinia pseudotuberculosis
MRLFAQLGWYFRREWHRYVGAVLLLIIIAILQLIPPKLVGVIVDGISTKQMSTNMLLVWIGVMLATAVVVYLLRYVWRVLLFGASYQLAVELRSDFYRQLSRQTPGFYSRHRTGDLMARATNDVDRVVFAAGEGVLTLVDSLVMGCAVLIVMSTQISWQLTLLSLLPMPIMAIVIKYYGDQLHQRFKSAQGAFSLLNNQAQESLTSIRMIKAFGLEDRQSQQFAQVAVETGAKNMYVARIDARFDPTIYIAIGIANLLAIGGGSWMVVNNSITLGQLTSFVMYLGLMIWPMLALAWMFNIVERGSAAYSRIRSLLDEAPVVKDGHITLSDVRDTLAVNIRHFCYPGSDQPALHNVVLTLVPGAMLGLCGPTGSGKSTLLALIQRQFDIDDGVICYQGHPLSDIRLNDWRGRLSVVSQTPFLFSDSVAGNIALGKPDATPAQIEQAARLACVHEDILRLPQGYDTEVGERGVMLSGGQKQRISIARALLLDAEILILDDALSAVDGQTEHEILKNLREWGEQRTVIISAHRLSALTEASEILVMQHGGVMQRGPHSLLVNQTGWYREMYRYQQLEAALDDGEQEVEADE

SEQ ID NO: 60 - protein, 146 aa, Yersinia pseudotuberculosis
MDISGFGTIVNIRASKTFPAGFNVTQFADDADPLDSPSQQLADVGMGLNGDMVSWSVAQVLQVTLNITPNSDDDRNLAILAEANRIAKGKRSVNDEITMSISYPSGESRTLSGGVITDAMIGNSVSSAGRLKSKPYIFKFENQVIA

SEQ ID NO: 61 - protein, 228 aa, Yersinia pseudotuberculosis
MAITNSLTESDYGITGTTGTSSTTGSSSQDLQNSFLTLLVAQLKNQDPTNPMENNELTTQLAQINTVSGIEKLNTTLGAITGQIDSSQSLYATSLIGRGVMVPGTNIFTGSTDGTVSTTPFGLELQRPADKVTATISDSNGQVVRTIEIGGLNAGVHSFTWDGSLDAGGNAPDGAYTVAITASNGGESLVATPLNYAIVNGVTRGADGSKLDLGLAGTITLDEVRQIL

SEQ ID NO: 62 - protein, 368 aa, Yersinia pseudotuberculosis
MNTEASQDQTVTETPGVRLRQARESLGLTQQTVAERLCLKVSTIRDIEEDNAQANLASTFHRGYIRSYAKLVHLPEDELLPILEKQAPVRAAKVAPMQSFSLGKKHKKRDGWLMSFTWLIVLVVLGLTGAWWWQNHQAQQAEIANMVDQSSAQLSQNGGQPVPLTDDNSDAIAPTDAPAPVANGQPVPLTNHSTSAVTNSATTSSATTSSVPTTSSVPKTTLVPKTTLVPKTNSTEPVDTANTNTTMHQEGAASAAVSPSQVPQLGMPTDQPPLPTADAGVSGSASSVGALVMNFTADCWLQVVDATGKTLFSGIQKGGAVLNLAGKAPYKLTIGAPGALTISYQGNPVDLSKFIKANRVARLTVGVE

SEQ ID NO: 63 - protein, 333 aa, Yersinia pseudotuberculosis
MNAMTEKKVLLEVADLKVYFNIQDGKQWFWQPSKTLKAVDGVTLRLYEGETLGVVGESGCGKSTFARAIIGLVKATGGSVAWLGKDLLNMSDADWRATRSDIQMIFQDPLASLNPRMTIGEIIAEPLRTYHPKISRQEVKDKVKAMMLKVGLLPNLINRYPHEFSGGQCQRIGIARALILEPKLVICDEPVSALDVSIQAQVVNLLQQLQREMGLSLIFIAHDLAVVKHISDRVLVMYLGHAVELGTYDEVYHNPQHPYTKALMSAVPIPDPDKERVKVIQLLEGELPSPIDPPSGCVFRTRCPIAGTECAKTRPLLEGSFRHAVSCLKVDPL

SEQ ID NO: 64 - protein, 289 aa, Yersinia pseudotuberculosis
MSTYLIGDIHGCLDELLALLAQVNFDPQQDTLWLTGDLVARGPASLDVLRYVRSLGPAVRMVLGNHDLHLLAVYAGISRNKPKDRITPLLDAPDADELINWLRRQPVLQVDDQLKLIMAHAGITPQWDIETAQMCAREVEAVLSSDSYPLFLDAMYGDMPNNWSPELTGLARLRFSTNALTRMRFCFPNGQLDMICKDTPENAPAPLKPWFDLPRLVDPEYSIIFGHWASLEGKGVPEGIYGLDTGCCWGGDLTLLRWEDKRYFTQRAFKAEAEINNNNGFAAGEEVQH

SEQ ID NO: 65 - protein, 373 aa, Yersinia pseudotuberculosis
MDYQLDLDWPDFLQRYWQKRPVILKRGFKNFIDPLSPDELAGLAMENEVDSRLVSHEDGRWHVSHGPFESFDHLGENNWSLLVQAVDHWHEPAAALMRPFRPLSDWRMDDLMISFSVPGGGVGPHFDQYDVFIIQGSGRRRWRVGEKTEMKQHCPHPDLLQVGPFDAIIDEEMEPGDILYIPPGFPHEGYSLENALNYSVGFRAPSGRELVSGFADYVLARELGSYRYSDPDLQLREHPAEVLPSEVDKLRTMMLDLVQQPEHFQNWFGEFISQSRHELDLAPPEPPYQTGDIYELLKQGDELQRLSGLRVLRVGDRCFANGELIDTPHLQAANALCQHFSVNAEMLGDALEDPSFLAMLAALVNSGYWYFND

SEQ ID NO: 66 - protein, 199 aa, Yersinia pseudotuberculosis
MYLSKPSIIYGFHGMDEDAALPILLKKDNFKHSNNSYDWLGNGVYFWENNYERAIQYAIEDKARKNSRIKKPFVLGAIIDLGNCLDLFDQKHIDFLKFSFEEMLGSLNSQKKEIPVNKKFGNSDFDFRCRELDCAVIRYAHTLAKKEGKPFDSVRAAFLEGNPLYEGAKFYEKNHIQIAVLNPNCIKGVFYPREKVVYP

SEQ ID NO: 67 - protein, 163 aa, Yersinia pseudotuberculosis
MDNIHRLFLALAFSAPLVTGAAHANSLLDSVKSTAEQYGKSAGSSSSLPSMSSLTNLLNGGDKALSAGTMNNATGILQYCVQNNVLSSDGTTAIKDQLMSKLGISGTEAAKSTDYEEGLGGLLKTSEGKSVNLNDLGTGQITEKIKTKACDLVLKQGKSFLLK

SEQ ID NO: 68 - protein, 321 aa, Yersinia pseudotuberculosis
MRIGHFQLTNCLIAAPMAGITDRPFRALCHGMGAGMAVSEMLSSNPEVWRTDKSRLRMVHVDEPGIRNVQIAGNDPDEMAAAARINVASGAQIIDINMGCPAKKVNRKLAGSALLQHPDLVKQILSAVVNAVDVPVTLKIRTGWSPEHRNCIEIAQLAENCGIQALTIHGRTRSCLFNGEAEYDSIRAVKQTVSIPVIANGDITDPHKARAVLDYTGADALMIGRAAQGRPWIFREIQHYLDTGELLPPMPLGEVQRLLDGHIRELHDFYGPGKGFRIARKHVSWYLQEHAPNDQFRRTFNAIEDASEQLEALEAYFENLA

SEQ ID NO: 69 - protein, 65 aa, Yersinia pseudotuberculosis
MMNFMTKAYVTAQVKAQAFAQDNRGSIIEYVMIIALAGVFIGLAKPELTDIITDTVAKTKAAIAP

SEQ ID NO: 70 - protein, 134 aa, Yersinia pseudotuberculosis
MSLLNIFDIAGSALSAQSQRMNVSASNLANADSVTGPDGQPYRAKQVVFQVAAQPGQETGGVRVAQIVDDPSPDRLVYQPGNPLADAKGYVRMPNVDVTGEMVNTISASRSYQANVEVLNTTKSLMLKTLTLGQ

SEQ ID NO: 71 - protein, 258 aa, Yersinia pseudotuberculosis
MLIIISPAKTLDYQSPLATTKFSQPEMLDKSQALIEICRELTPAQISSLMGISDKLAGLNAARFSEWQPDFTPANARQAILAFKGDVYTGMQAESFSEADFDFAQQHLRMLSGLYGLLRPLDLMQPYRLEMGTKLANPRGKDLYAFWGDQITEKLNQALELQGDNILINLASDEYFKAVKPAKLSGSLIKPVFLDEKNGKYKIISFYAKKARGLMSRFIIQNKLTKPEQLVDFNLEGYEFDAGLSAKNELVFKRAEQH

SEQ ID NO: 72 - protein, 316 aa, Yersinia pseudotuberculosis
MLLNNITPVNKSLTLQDLLGILSHSSAISNVANGIYVESEILEVGSWLSAYAANKDEIFSQIITELENPYQFQLENDIQAPSFILYSNERITIRLVMWLPLQGKLDRTPYSYEEAHDHNFDFWTVNFFGGGYRTRLYDYDYDKVSGVNNEVVELNCYGDKILSPNTVMFYFRSKDVHTQYPPDELSVSLNLIVRPIKSKHQYEFQIDSDALEGKIEARIKKGRYERYAFQNVLYNGLLSLENEKSRQLVHKVSLCNHREEIRLIAYEALLKHAQKKGNVSDIKSISEQAFKDQSLYIKNKISHSIGSMPCMSPKPR

SEQ ID NO: 73 - protein, 466 aa, Yersinia pseudotuberculosis
MQQHFHFDAIVIGSGPGGEGAAMGLVKQGARVAVIERYNNVGGGCTHWGTIPSKALRHAVSRIIEFNQNPLYSDNARTIKSSFADILNHADRVINQQTRMRQGFYDRNHCHMFSGDASFIDANTVNVRYADGTSDTLQADNIVIATGSRPYRPVNVDFNHERIYDSDTILQLSHEPQHVIIYGAGVIGCEYASIFRGLSVKVDLINTRDRLLAFLDQEMSDALSYHFWNNGVVIRHNEEFEQIEGTTDGVIVHLKSGKKVKADCLLYANGRTGNTSGLGLENIGLEADSRGLLKVNSMYQTALSHVYAVGDVIGYPSLASAAYDQGRIAAQAMIKGEANVHLIEDIPTGIYTIPEISSVGKTEQELTAMKVPYEVGRAQFKHLARAQIVGMDTGSLKILFHRETKQILGIHCFGERAAEIIHIGQAIMEQKGEGNTLEYFVNTTFNYPTMAEAYRVAALNGLNRLF

SEQ ID NO: 74 - protein, 73 aa, Yersinia pseudotuberculosis
MKAHVSSREYQDNGNKRIYTLMDGSVVIEYPNLPGKSRFNFFNHCGNTVHKNQQRVAMKQAVEHHKKQWKVKP

SEQ ID NO: 75 - protein, 823 aa, Yersinia pseudotuberculosis
MSTSFLVGAQRYSFDPNLLVDGNNNTDTSLFEQGNELPGTYLVDIILNGNKVDSTNVTFHSEKSPSGEPFLQSCLTKEQLSRYGVDVDAYPELSPALKNSQTNPCVNLAAIPQASEEFQFYNMQLVLSIPQAALRPEGEVPIERWDDGITAFLLNYMANISETQFRQNGGYRRSQYIQLYPGLNLGAWRVRNATNWSQSGDRGGKWQSAYTYATRGIYRLKSRVTLGESYTPGDFFDSIPFRGVMLGDDPNMQPSNQRDFIPVVRGIARSQAQVEIRQNGYLIYSTVVPPGPFELSDVIPSKSGSDLHVRVLESNGASQAFIVPYEVPAIALRKGHLRYNLVAGQYRPANADVETPPVAQATVAYGLPWNLTAFIGEQWSRHYQATSAGLGVLLGEYGALSSSITQATSQYHHQQPVKGQAWEVRYNKTLQASDTSFSLVNSQYSTNGFSTLSDVLQSYRQSGSGDNRDKIDENSRSRDLRNQISAVIGQSLGKFGYLNLNWSRQVYRGPIPAKNSLGIHYNLNVGNSFWALSWVQNANENKNDRILSLSVSIPLGGHHDTYASYRMTSSNGSNDHEIEMYGQAFDSRLSWSVRQAEHYGQPNSGHNSGSLRLGWQGSYGNIAGNYYYTPSIRQLSADVSGGAIIHRHGLTLGPQINGTSVLVEVPGVGGVTTTEDRRLKTDFRGYSIVSGLSPYQEHDIVLETADLPPDAEVAKTDTKVLPTEGAIVRASFSPQIGAKALMTITRANGQTIPFGAMASLVNQSANAAIVDEGGKAYLTGLPETGQLLVQWGKDAGQQCRVDYQLSPAEKGDAGLYMLSGVCH

SEQ ID NO: 76 - protein, 474 aa, Yersinia pseudotuberculosis
MSLSRRQFIQAAGLALGAGSLPLRAQASSTQQPQLPVPPLLESRRGQPLFLTLQRAHWAFSGNKKAAVWGINGMYLGPTVRVFNGDDVKLIYSNRLTEPVSMTISGLQVPGTLMGGEARMIHPGEDWSPVLPVRQPAANCWYHANTPNRMAPHVYNGLAGMWLVEDAVSKAMPLPSHYGVDDFPLIIQDKRLDNFGVPEYNPPAKGGFVGDTLLVNGAQSPFVEVSRGWVRLRLLNASNARRYTLQLSDGRPLYVVASDQGFLPAPVAVQQLSLAPGERREVVIDMSQGNEVSITAGESAGIMDRLRGLFEPSSILISTLVLTLKPTGLLPLVTDNLPMRLLADQIIEGSVIRSREFQLGDNLPGINGAIWDMNRVDVQAQQGTWERWIIHADMPQAFHIQGVSFLVKSVNGAAAMAEDRGWKDTVWVDGTVELWVYFNQVSSPQFPFLFYSQTLEMADRGSAGQLVTVAAPTL

SEQ ID NO: 77 - protein, 969 aa, Yersinia pseudotuberculosis
MSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNSVGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGRSLVSSLEASRQCQGSDMSAVLESSRATNGTRAPDGTLWGEWGSLTAYSSDWQSGEYWVKKTSTDFETMNMDTGALQPGPAYLAFPLCALSI

SEQ ID NO: 78 - protein, 158 aa, Yersinia pseudotuberculosis
MKMKCFAKNALAVTTLMIAACGMANASTVINSKDVSGEVTVKQGNTFHVDFAPNTGEIFAGKQPGDVTMFTLTMGDTAPHGGWRLIPTGDSKGGYMISADGDYVGLYSYMMSWVGIDNNWYINDDSPKDIKDHLYVKAGTVLKPTTYKFTGRVEEYVF

SEQ ID NO: 79 - protein, 273 aa, Yersinia pseudotuberculosis
MKNLFFSAYKKVFSYITSIVIFMVSLPYAYSQDVVVNTTKHLFTVKIGTTRVIYPSSSTKGVSVSVANPQDYPILVQTQVKDEDKTSPAPFIVTPPLFRLDAGLQGRVRIIRTGGKFPEDRETLQWLCLTGIPPKNGDAWGNTQNNPKNSSPTMDIQMSISTCIKLLFRPDKVKGDPTDSADSLTWRYKGNYLEVNNPTPFYMNFYSLRIGDEKINLSDLGSKDEIKNGSYVPPFSSRDFIIPVKNKGKATEVFWQVINDNGGVSREFKSTVQ

SEQ ID NO: 80 - protein, 273 aa, Yersinia pseudotuberculosis
MDQKFAVFGNPISHSKSPRIHTLFSEQTGIEHRYGKVLAPSEAFENTLVSFFADGAQGANITTPFKERAYDQCDELTDRASLAGAVNTIKRLEDGRLLGDNTDGIGLLSDLERQNLIRTTDHILLVGAGGAARGVILPLLSYGCTVVVTNRTHTRAQQLAKVFNHIGDIDVCEMSELAGQRFDLVINATASGLHGEVPNLPAAILTSQTRCYDMFYQAGTTPFLAWAQRLGLADYADGLGMLVGQAAHAFKLWHGVMPEITPVLAQLRSELGK

SEQ ID NO: 81 - protein, 1296 aa, Yersinia pseudotuberculosis
MEILRGSPALSAFRITKLLSRCQDAHLPVSDIYAEYVHFADVSAPLSADEHARLQRLLQYGPSLPEHPPAGRLLLVTPRPGTISPWSSKATDIAHNCGLSQILRLERGLAFSIQGPNLNEGQWKQLAALLHDRMMETVFTDLQQAEQLFSHHQPAPVQRVDILGQGRSALEQANIKLGLALAQDEIDYLLTAFTGLGRNPTDIELYMFAQANSEHCRHKIFNADWVIDGVAQPKTLFKMIKNTFEHTPDYVLSAYKDNAAVMEGSQVGRFYATAEKGIYDYHQEEAHILMKVETHNHPTAISPWPGAATGSGGEIRDEGATGRGAKPKAGLVGFSVSNLRIPGFEQPWEENFGKPDRIVTALDIMTEGPLGGAAFNNEFGRPALLGYFRTYEERVNSHNGIELRGYHKPIMLAGGLGNIRADHVQKGEITVGAKLVVLGGPSMNIGLGGGAASSMASGQSDADLDFASVQRDNPEMERRCQEVIDRCWQLGEYNPILFIHDVGAGGLSNAMPELVNDGGRGGRFELRDILNDEPGMSPLEVWCNESQERYVLAVAPAQMALFDEICRRERAPYAVIGEATEEKHLLLNDRHFGNQPIDMPLDVLLGKTPKMLRDVTRLQAKGDALQRADISLAEAVKRIMHLPAVAEKTFLITIGDRTVTGMVTRDQMVGPWQIPVADCAVTSASLDSYYGEAMSLGERAPVALLDFAASARLAVGEALTNIAATQIGELKRIKLSANWMSAAGHPGEDAGLYDAVRAVGEELCPALEITIPVGKDSMSMKTRWQEGHEQREMTSPLSLVITAFARIEDVRRTVTPQLRTDKGDNALLLIDLGAGHNALGATALTQVYRQLGDKPADVRNVQQLAGFFNAMQRLVADQHLLAYHDRSDGGLLVTLAEMAFAGHCGVTVDIQSLGNDALAALFNEELGAVIQVRAEQRADVEKLLADHGLANCVHYLGRAVAGDTFDIRSGTDVVYSEKRSTLRLWWAETSWQMQRLRDNPDCADQEHQAKQDESDPGLNVKLTFDPAEDIAAPFILKQARPKVAVLREQGVNSHVEMAAAFHRAGFDVVDVHMSDLLAGRTDLQSFQTLVACGGFSYGDVLGAGEGWAKSILFNDRVRDEFEAFFHRPTTLALGVCNGCQMMSNLRELIPGAEHWPRFVRNLSDSFEARFSLVEVASSPSLFMQDMVGSRMPIAVSHGEGQVEVRDAAHLAALEQSNLVALRFVNNHGVVTEQYPANPNGSANGITAVTSVSGRATVMMPHPERVFRTVSNSWHPEEWGEDSPWMRMFRNARKQLG

SEQ ID NO: 82 - protein, 362 aa, Yersinia pseudotuberculosis
MEKITVTLGERSYPITIAAGLFNDPASFKPLKAGDQVMLVTNQTLAPLYLDSLRAVLEHGGIKVDQVILPDGEQYKSLSVMEQVFSALLEKPHGRDTTLVALGGGVVGDLTGFAAACYQRGVRFIQVPTTLLSQVDSSVGGKTAVNHPLGKNMIGAFYQPASVVVDLNCLKTLPPRELASGLAEVIKYGIILDAAFFDWLENNIDALLALDMSALAYCIRRCCELKADVVAADEREESGARALLNLGHTYGHAIEAEMGYGVWLHGEAVAAGMVMAAQTSRRLGQLSVSDVERIKKLLLRAGLPVCGPKEMAPESYLPHMMRDKKVLAGELRLVLPTAIGKSEIRGGVAHDMVLASIADCRP

SEQ ID NO: 83 - protein, 529 aa, Yersinia pseudotuberculosis
MQQRRPIRRALLSVSDKAGIIEFAQALSQRGIELLSTGGTARLLADAGLPVTEVSDYTGFPEMMDGRVKTLHPKVHGGILGRRGQDDGIMAQHGIQPIDIVVVNLYPFAQTVARPDCSLEDAVENIDIGGPTMVRSAAKNHKDVAIVVKSSDYPAIITELDNNDGSLTYPTRFNLAIKAFEHTAAYDSMIANYFGTLVPPYHGDTEQPSGHFPRTLNLNYIKKQDMRYGENSHQQAAFYIEEDVKEASVATAQQLQGKALSYNNIADTDAALECVKEFSEPACVIVKHANPCGVAIGDSILAAYERAYQTDPTSAFGGIIAFNRELDAATANAIISRQFVEVIIAPTVSSDALALLAAKQNVRVLTCGQWQARSAGLDFKRVNGGLLVQERDLGMVTAADLRVVSKRQPTEQELRDALFCWKVAKFVKSNAIVYARDNMTIGIGAGQMSRVYSAKIAGIKAADEGLEVAGSAMASDAFFPFRDGIDAAAAVGITCVIQPGGSIRDDEVIAAADEHGIAMIFTDMRHFRH

SEQ ID NO: 84 - protein, 428 aa, Yersinia pseudotuberculosis
MLESLTLQPIALVNGTVNLPGSKSVSNRALLLAALAEGTTQLNNVLDSDDIRHMLNALQALGVNFRLSADRTCCEVDGLGGKLVAEQPLSLFLGNAGTAMRPLAAVLCLGNSDIVLTGEPRMKERPIGHLVDALRQGGAQIDYLEQENYPPLRLRGGFRGGELTVDGRVSSQFLTALLMTAPLAEQDTTIRIMGDLVSKPYIDITLHLMKAFGIDVGHENYQIFHIKGGQTYRSPGTYLVEGDASSASYFLAAAAIKGGTVRVTGIGKKSVQGDTKFADVLEKMGAKVTWGDDYIECSRGELQGIDMDMNHIPDAAMTIATTALFATGPTTIRNIYNWRVKETDRLTAMATELRKVGAEVEEGEDYIRVVPPVQLTAADIGTYDDHRMAMCFSLVALSDTPVTILDPKCTAKTFPDYFEQFARLSQLA

SEQ ID NO: 85 - protein, 268 aa, Yersinia pseudotuberculosis
MERYQQLFKQLAAKKEGAFVPFVQLGDPSPAMSLNIIDTLIAAGADALELGIPFSDPLADGPTIQNAALRAFAAGVTPAICFEILAEIRQKHPTIPIGLLMYANLVFHNGIDHFYQRCAEVGVDSVLIADVPFEESAPFRAAALRHGIAPIFICPPNADGDLLREIASHGRGYTYLLSRAGVTGAENHGQLPLNHLVDKLREYNAAPALQGFGISEPAQVKASLAAGAAGAISGSAIVKIIEKNVAQPVEMLVQLTRFVTEMKAATRS

SEQ ID NO: 86 - protein, 355 aa, Yersinia pseudotuberculosis
MSQKFLFIDRDGTIIAEPPTDYQVDRLDKLALEPDVIPALLALQKADYKLVMITNQDGLGTSSFPQETFDPPHNLMMQILTSQGINFEQILICPHLPADNCTCRKPKTALVESYLADGVMNSATSYVIGDRETDLQLAENMGISGLRYQRDGLNWTQIAKQLTQRDRHAYVNRVTKETAIDVNVWLDREGGSKIKTGVGFFDHMLDQIATHGGFRMDIQVSGDLYIDDHHTVEDTALALGEAINIALGDKRGIGRFGFVLPMDECLARCALDISGRPHLEYKAEFNYQRVGDLSTEMVEHFFRSLSYAMACTLHLKTKGRNDHHRVESLFKVFGRTLRQAIRVEGNTLPSSKGVL

SEQ ID NO: 87 - protein, 428 aa, Yersinia pseudotuberculosis
MNILIIGNGGREHALGWKAAQSPLADKIYVAPGNAGTALEPTLENVDIAATDIAGLLAFAQSHDIGLTIVGPEAPLVIGVVDAFRAAGLAIFGPTQAAAQLEGSKAFTKDFLARHNIPSAEYQNFTDVEAALAYVRQKGAPIVIKADGLAAGKGVIVAMTLEEAETAVNDMLAGNAFGDAGHRIVVEEFLDGEEASFIVMVDGENVLPMATSQDHKRVGDGDTGPNTGGMGAYSPAPVVTDDVHQRVMDQVIWPTVRGMAAEGNIYTGFLYAGLMISADGQPKVIEFNCRFGDPETQPIMLRMRSDLVELCLAGTQGKLNEKTSDWDERPSLGVVLAAGGYPADYRQGDVIHGLPQQEVKDGKVFHAGTKLNGNHEVVTNGGRVLCVTALGETVAQAQQYAYQLAEGIQWEGVFCRKDIGYRAIARGK

SEQ ID NO: 88 - protein, 237 aa, Yersinia pseudotuberculosis
MQKLAELYRGKAKTVYTTENPDLLVLEFRNDTSALDGQRIEQFDRKGMVNNKFNHFIMTKLEEAGIPTQMERLLSDTEVLVKKLEMIPVECVIRNRAAGSLVKRLGIEEGLSLNPPLFDLFLKNDAMHDPMVNESYCKTFGWATEAQLARMKELSYLANDVLSKLFDDAGLILVDFKLEFGLFNGEVVLGDEFSPDGSRLWDKKTLNKMDKDRYRQSLGGLIEAYEEVAHRIGVKLD

SEQ ID NO: 89 - protein, 462 aa, Yersinia pseudotuberculosis
MLLPVIMAGGAGSRLWPLSRALYPKQFLALTSDLTMLQETLLRLDGLPHLAPLVICNEEHRFIIAEQLRQKNLVHSGIVLEPVGRNTAPAIALAALRATMSGDDPLLLVLAADHVIQDKLAFIRAVQRAEPLAEAGKLVTFGIVPKSPETGYGYIRQGKQVVDGAYQVAAFVEKPDLITAERYLASGDYYWNSGMFAFKASRYLQELALHRPDILAACKQAIAGQHTDLDFIRLNEEAFSSCPSESIDYAVMEKTSDAVVVPLDAQWNDVGCWSALWEINTKDDHGNVIRGDVLMEDTNNSYVYSQNRLIATVGINDLVIVETKDAILVAHKDKVQNVKGIVGQLKLESRCEYLQHREVYRPWGSHDAIAEGIRYHVQHVTVKPGQRITTQIHYHRAEHWIVVSGTALVTIADKTIILCENESTFIPIGKPHSLENPGSIPLEIIEVQSGSYLGEDDIIRLE

SEQ ID NO: 90 - protein, 410 aa, Yersinia pseudotuberculosis
MTTLSPAIPVNRRSEMLCSIAALLLGISMPTNNQLMSVSLVLIIISLIINRKSLDFKPLLTSPLVYLPAAMFVLLALSLLYQNNSYGPDMVGKYKKLLYILPLALFFMNQPRLIKLFCTGFLVANAVILAGSLAVGVLHIPLSGVDPTNPTIFKLQITQNFFMALAALLWLVLAFQHQGWKRWGYSVLVVAASYSILFLVLGRTGYVALIVGLGVWLFFSLGNRQRLTLVVLGALAFAALIFIPNKATDRIVQGVDEIKVCMAASATDAADACNSSMGQRSAFVVEAARLIKESPILGHGAGGFYYENKEVDYKVNNPHNQYLLETIQSGVIGLFLFLAWVICCYRVIWQQTPALRNVLLAVLTSYMACNFFNSFLLDSSEGHLFMIFVAVLAGYSVSGSQSIAGKRLPT

SEQ ID NO: 91 - protein, 360 aa, Yersinia pseudotuberculosis
MSTELIYIFLFSMAFLFVARKVAIKIGLVDKPNYRKRHQGLIPLVGGISVFAGVCFAFLITNQQIPHFRLYLACAGLLVFVGALDDRFDISVKIRAFVQALVGIAMMAVAGLYLRSLGHAFGPWEMVLGPFGYVVTLFAVWAAINAFNMVDGIDGLLGGLSCVSFGAMGILLYQSGQMSLALWCFAMIATIIPYILLNLGLLGRRYKVFMGDAGSTLIGFTAIWILLQATQGNAHPINPVTALWIIAIPLMDMIAIMYRRLRKGMSPFSPDRQHIHHLIMRAGFTSRQAFVLITLAAALLAMIGVIGERLTFIPEWVMLALFLLAFLLYGYCIKRAWRVARFIKRFKRRMRRASKNKHES

SEQ ID NO: 92 - protein, 161 aa, Yersinia pseudotuberculosis
MKSWYLLYCKRGQILRAKEHLERQTVNCWTPIVAIEKIVRGKRIEVIEALFPNYLFAEFDPENIHTTTVSATRGVSHFVRFGTQPAVIPATVIADMQAHAVDKIIAPEVPKPGDIVKIIDGVFAGLQAIYTEPDGEARSMLLLNMLNSQIKHSLDNRQFEK

SEQ ID NO: 93 - protein, 337 aa, Yersinia pseudotuberculosis
MKIIYDGIINSLQRTGGITIYFKELVTRLPERYFDWYSYDVKLGDIGVDGIELKSRLLERYRDFSIKNVSDKSPDIFHSSYYRLPKFDIPIVTTVHDFTYEKFINGPAKWVHSWQKNRAVNNSDLIICVSENTAKDLQKYCSVSSEKIRIVHNGVSEKYHSITTVTSYTNKVIFVGARGGYKNFDIAVKAISKTPHLELSVVGGGAFTSKELSLLNHYLPGRYHGLGRLSDEALNEAYNSAYALLYPSSYEGFGIPILEAMSAGCPVISVNVSSIPEVAGDAAILVQKPTVDELVDGLLAVESERSKLIGYGMKQAAKFSWDKCYQETLDVYKELNK

SEQ ID NO: 94 - protein, 357 aa, Yersinia pseudotuberculosis
MINNSFWQGKRVFVTGHTGFKGGWLSLWLQTMGATVKGYSLTAPTVPSLFETARVADGMQSEIGDIRDQNKLLESIREFQPEIVFHMAAQPLVRLSYSEPVETYSTNVMGTVYLLEAIRHVGGVKAVVNITSDKCYDNKEWIWGYRENEAMGGYDPYSNSKGCAELVTSSYRNSFFNPANYGQHGTAVATVRAGNVIGGGDWALDRIVPDILRAFEQSQPVIIRNPHAIRPWQHVLEPLSGYLLLAQKLYTDGAEYAEGWNFGPNDADATPVKNIVEQMVKYWGEGASWQLDGNAHPHEAHYLKLDCSKAKMQLGWHPRWNLNTTLEYIVGWHKNWLSGTDMHEYSITEINNYMNTK

SEQ ID NO: 95 - protein, 319 aa, Yersinia pseudotuberculosis
MKIALIGGSGFIGTNLARLLIDNSVDFSILDKVKSDVYPERWVYCDVTDYDSLISTLIGHDLIINLAAEHKDNVNPISLYYQVNVEGAKNICRAADSLNIKNIVFTSSVAVYGFVEKDTDESGKYAPFNHYGKSKLEAEKVYDSWFNSSADKKLVTLRPTVVFGIGNRGNVYNLFKQIASGKFVMIGRGENEKSMAYVENIAAFLVLTLSFPAGYHLINYVDKPDFTMNELANVIYTCLGKKSKIVRVPYFFGLFAGYIFDLLAKITGKELPVSSIRIKKFCAKTQFSSKNIENYKFSAPYSLQDAVVKTISQEFMSEK

SEQ ID NO: 96 - protein, 373 aa, Yersinia pseudotuberculosis
MTKIALITGITGQDGSYLAEFLLEKGYEVHGIKRRASSFNTSRIDHIYQDRHETNPRFFLHYGDLTDTSNLIRLVQEIQPDEIYNLGAQSHVAVSFESPEYTADVDAMGTLRLLEAIRINGLEKKTRFYQASTSELYGLVQETPQRETTPFYPRSPYAVAKMYAYWITVNYRESYGMYACNGILFNHESPRRGETFVTRKITRAVANIALGLEKCLYLGNIDSLRDWGHAKDYVRMQWMMLQQDKPEDFVIATGKQITVREFVRMSAREAGIELEFSGEGVEEVATVVAINGNHISSVNIGDVIVRVDPRYFRPAEVETLLGDPTKAKKVLGWVPEITVEEMCAEMVASDLEQAKQHALLKANGFDVSISLEN

SEQ ID NO: 97 - protein, 321 aa, Yersinia pseudotuberculosis
MDKKRVFIAGHRGMVGSAIVRQLENRNDIELIIRDRTELDLMSQSAVQKFFATEKIDEIYLAAAKVGGIQANNNYPAEFIYQNLMIECNIIHAAHLAGIQKLLFLGSSCIYPKLAAQPMTEEALLTGVLEPTNEPYAIAKIAGIKLCESYNRQYGRDYRSVMPTNLYGENDNFHPENSHVIPALLRRFHEAKIRNDKEMVVWGTGKPMREFLHVDDMAAASVHVMELSDQIYQTNTQPMLSHINVGTGVDCTIRELAETMAKVVGFTGNLVFDSTKPDGTPRKLMDVSRLAKLGWCYQISLEVGLTMTYQWFLAHQNNFRK

SEQ ID NO: 98 - protein, 437 aa, Yersinia pseudotuberculosis
MSQEELRQQIAELVAQYAETAMAPKPFEAGKSVVPPSGKVIGTKELQLMVEASLDGWLTTGRFNDAFEKKLGEYLGVPYVLTTTSGSSANLLALTALTSPKLGVRALKPGDEVITVAAGFPTTVNPTIQNGLIPVFVDVDIPTYNVNASLIEAAVSDKTKAIMIAHTLGNLFDLAEVRRVADKYNLWLIEDCCDALGSTYDGKMAGTFGDIGTVSFYPAHHITMGEGGAVFTQSAELKSIIESFRDWGRDCYCAPGCDNTCKKRFGQQLGSLPFGYDHKYTYSHLGYNLKITDMQAACGLAQLERIEEFVEKRKANFKYLKDALQSCADFLELPEATENSDPSWFGFPITLKEDSGVSRIDLVKFLDEAKVGTRLLFAGNLTRQPYFHDVKYRVVGELTNTDRIMNQTFWIGIYPGLTHDHLDYVVSKLEEFFGLNF

SEQ ID NO: 99 - protein, 420 aa, Yersinia pseudotuberculosis
MSFETISVIGLGYIGLPTAAAFASRKKKVIGVDVNAHAVETINRGAIHIVEPDLDKVVKIAVEGGYLQAVTKPQAADAFLIAVPTPFKGDHEPDMIFVESAAKSIAPVLKKGDLVILESTSPVGATEQMAQWLAEARPDLSFPQQAGEAADINIAYCPERVLPGQVMVELIQNDRVIGGMTPKCSARASALYKIFLEGECVVTNSRTAEMCKLTENSFRDVNIAFANELSLICDEQGINVWELIRLANRHPRVNILQPGPGVGGHCIAVDPWFIVSQNPQLARLIHTARLVNDGKPLWVVDRVKAAVADCLAASDKRASEVKIACFGLAFKPDIDDLRESPAVGVARLIAEWHVGETLVVEPNVEQLPKSLMGLVTLKDTATALQQADVLVMLVDHKQFKAIKPEDIKQQWIVDTKGVWR

SEQ ID NO: 100 - protein, 301 aa, Yersinia pseudotuberculosis
MKQVGLRIDVDTYRGTQYGVPSLLTVLEKHDIRASFFFSVGPDNMGRHLWRLFRPRFLWKMLRSNAASLYGWDILLAGTAWPGKKIAKDFGPLMKAAAMAGHEVGLHAWDHQGWQANVASWSQQQLTEQVQRGVDTLQQSIGQPISCSAAAGWRADERVLAVKQQFDFSYNSDCRGTHPFRPLLPNGSLGSVQIPVTLPTYDEVVGGEVQAENFNDFIIDAILRDSGVSVYTIHAEVEGMSQAAMFEQLLMRAKQQDIEFCPLSKLLPSDLQLLPVGKVIRATFPGREGWLGCQSDIKDAE

SEQ ID NO: 101 - protein, 249 aa, Yersinia pseudotuberculosis
MKISIITATYNSASTIVETLDSLNEQTYDNIEHIIIDGGSTDNTLELVKAYGKRVSIVISEKDEGIYDALNKGISVATGDIIGILHSDDMFAYIDAVSDIAKVFFDNTVDACYGDLSYISRSGDNRVVRTWIAGDYSESKYKYGWMPPHTTFYMKNALYKKLGGYDTTLKIAADYDAMLRYTLVAKINIVYIPKILIYMKVGGVSTRLSQKFESLKDELIVMKRYNLGGVMTFMKKKIYKLPQFFKIVK

SEQ ID NO: 102 - protein, 285 aa, Yersinia pseudotuberculosis
MKILITGVSGYLGSQLANALMLEHEVAGTVRAGSVCNRITDIGNVNLINVTDSGWIDKVLSFSPDVVINTVALYGRKGELLSELVDANIQFPLRILEMLVSTGKGLFFQCGTSLPADVSQYALTKNQFTELAREYCNKFSGKFIELKLEHFFGPFDDSTKFTTYVINSCRSHSDLKLTAGLQRRDFIYINDLINAFKIMISKSESLISGESISIGSGHAVTIKEFVETVAKMTSYQGNLQFGAIPTRENELMYSCASLARIQELGWLCQYSLNSAIKDTLNRMRV

SEQ ID NO: 103 - protein, 1195 aa, Yersinia pseudotuberculosis
MRLFAQLGWYFRREWHRYVGAVLLLIIIAILQLIPPKLVGVIVDGISTKQMSTNMLLVWIGVMLATAVVVYLLRYVWRVLLFGASYQLAVELRSDFYRQLSRQTPGFYSRHRTGDLMARATNDVDRVVFAAGEGVLTLVDSLVMGCAVLIVMSTQISWQLTLLSLLPMPIMAIVIKYYGDQLHQRFKSAQGAFSLLNNQAQESLTSIRMIKAFGLEDRQSQQFAQVAVETGAKNMYVARIDARFDPTIYIAIGIANLLAIGGGSWMVVNNSITLGQLTSFVMYLGLMIWPMLALAWMFNIVERGSAAYSRIRSLLDEAPVVKDGHITLSDVRDTLAVNIRHFCYPGSDQPALHNVVLTLVPGAMLGLCGPTGSGKSTLLALIQRQFDIDDGVICYQGHPLSDIRLNDWRGRLSVVSQTPFLFSDSVAGNIALGKPDATPAQIEQAARLACVHEDILRLPQGYDTEVGERGVMLSGGQKQRISIARALLLDAEILILDDALSAVDGQTEHEILKNLREWGEQRTVIISAHRLSALTEASEILVMQHGGVMQRGPHSLLVNQTGWYREMYRYQQLEAALDDGEQEVEADEMNNAQQLWPTLKRLLSYGSPYRKPLGLAVLMLWVAAAAEVSGPLLISYFIDHVVAKGTLPLGLVSGLALAYLLLQLLAATLHYFQALLFNRAAVGVVQRLRIDVMDAALRQPLSAFDTQPVGQLISRVTNDTEVIKDLYVMVVSTVLKSAALISAMLVAMFSLDWRMALISICIFPAVLVVMTIYQRYSTPIVRRVRSYLADINDGFNEVINGMGVIQQFRQQARFGERMASASRAHYVARMQTLRLEGFLLRPLLSLFSALVLCGLLLLFGFSPEGSVGVGVLYAFINYLGRLNEPLIELTSQQSIMQQAVVAGERIFDLMDRAQQDYGSDNIPLSSGRIQVENVSFAYRSDKMVLHNISLSVPSRGFVALVGHTGSGKSTLANLLMGYYPIQQGEILLDGRPLSRLSHQVLRQGVALVQQDPVVLADSFFANITLGRDLSEQQVWEALETVQLAPLVRTLPDGLYSLLGEQGNTLSVGQKQLLAMARVLVQAPQILILDEATANIDSGTEQAIQRALQVIRKNTTLVVIAHRLSTIVEADSILVLHRGVAVEQGNHQALLAARGRYYQMYQLQLVSEDLAAIDQEAIDKGSIDQSTIDQAGMSVS